SEQUENCE LISTING (116 sequences)

SEQ ID NO: 1 - DNA, 28 bp, Artificial Sequence
  synthetic
gatcctaggtctgagattctcaggatct

SEQ ID NO: 2 - DNA, 24 bp, Artificial Sequence
  synthetic
agatcctgagaatctcagacctag

SEQ ID NO: 3 - DNA, 28 bp, Artificial Sequence
  synthetic
gatcctaggtctgagattctcaggatct

SEQ ID NO: 4 - DNA, 30 bp, Artificial Sequence
  synthetic
ctaggatccagactctaagagtcctagann

SEQ ID NO: 5 - DNA, 27 bp, Artificial Sequence
  synthetic
actctagatctgagattctcaggatct

SEQ ID NO: 6 - DNA, 24 bp, Artificial Sequence
  synthetic
agatcctgagaatctcagatctag

SEQ ID NO: 7 - DNA, 20 bp, Artificial Sequence
  synthetic
adtgcaccaaagtaaccctg

SEQ ID NO: 8 - DNA, 17 bp, Artificial Sequence
  synthetic
cagggttactttggtgc

SEQ ID NO: 9 - DNA, 72 bp, Artificial Sequence
  synthetic
tggaagcgataaaactctgcaggttggatacgccaatcatttttatcgaagcgcgcataaatttgagcagat

SEQ ID NO: 10 - DNA, 36 bp, Artificial Sequence
  synthetic
aacccacatctacagaccctgaagaaacagtcaagt

SEQ ID NO: 11 - DNA, 14 bp, Artificial Sequence
  synthetic
gatgtgggtttgca

SEQ ID NO: 12 - DNA, 36 bp, Artificial Sequence
  synthetic
aacccacatctacagaccaagctgaaacagtcgcca

SEQ ID NO: 13 - DNA, 18 bp, Artificial Sequence
  synthetic
gactgaagtgatctccct

SEQ ID NO: 14 - DNA, 18 bp, Artificial Sequence
  synthetic
agtcgaagtgatctccct

SEQ ID NO: 15 - DNA, 18 bp, Artificial Sequence
  synthetic
cgcgagggagatcacttc

SEQ ID NO: 16 - DNA, 36 bp, Artificial Sequence
  synthetic
aacccacatctacagaccaagctgaaacagtctact

SEQ ID NO: 17 - DNA, 36 bp, Artificial Sequence
  synthetic
aacccacatctacagaccaagctgaaacagtcattt

SEQ ID NO: 18 - DNA, 36 bp, Artificial Sequence
  synthetic
aacccacatctacagaccaagctgaaacagtctcat

SEQ ID NO: 19 - DNA, 36 bp, Artificial Sequence
  synthetic
aacccacatctacagaccaagctgaaacagtcttct

SEQ ID NO: 20 - DNA, 36 bp, Artificial Sequence
  synthetic
aacccacatctacagaccaagctgaaacagtcaata

SEQ ID NO: 21 - DNA, 36 bp, Artificial Sequence
  synthetic
aacccacatctacagaccaagctgaaacagtccgat

SEQ ID NO: 22 - DNA, 36 bp, Artificial Sequence
  synthetic
aacccacatctacagaccaagctgaaacagtccaca

SEQ ID NO: 23 - DNA, 36 bp, Artificial Sequence
  synthetic
aacccacatctacagaccaagctgaaacagtcgaaa

SEQ ID NO: 24 - DNA, 28 bp, Artificial Sequence
  synthetic
gactctagatctgagattctcaggatct

SEQ ID NO: 25 - DNA, 30 bp, Artificial Sequence
  synthetic
agatcctgagaatctcagatctagagtcnn

SEQ ID NO: 26 - DNA, 36 bp, Artificial Sequence
  synthetic
aacccacatctacagaccaagctgaaacagtcagta

SEQ ID NO: 27 - DNA, 36 bp, Artificial Sequence
  synthetic
aacccacatctacagaccaagctgaaacagtcctga

SEQ ID NO: 28 - DNA, 24 bp, Artificial Sequence
  synthetic
actctagatctgagattctcagga

SEQ ID NO: 29 - DNA, 24 bp, Artificial Sequence
  synthetic
agatcctgagaatctcagatctag

SEQ ID NO: 30 - DNA, 18 bp, Artificial Sequence
  synthetic
adtgcaccaaagtaccct

SEQ ID NO: 31 - DNA, 17 bp, Artificial Sequence
  synthetic
cagggttactttggtgc

SEQ ID NO: 32 - DNA, 12 bp, Artificial Sequence
  synthetic
ttttagtctact

SEQ ID NO: 33 - DNA, 12 bp, Artificial Sequence
  synthetic
ttttagtcattt

SEQ ID NO: 34 - DNA, 12 bp, Artificial Sequence
  synthetic
ttttagtcgaaa

SEQ ID NO: 35 - DNA, 12 bp, Artificial Sequence
  synthetic
ttttagtcttct

SEQ ID NO: 36 - DNA, 12 bp, Artificial Sequence
  synthetic
ttttagtcaagt

SEQ ID NO: 37 - DNA, 12 bp, Artificial Sequence
  synthetic
ttttagtcgcca

SEQ ID NO: 38 - DNA, 28 bp, Artificial Sequence
  synthetic
agatcctgagaatctcagatctagagtc

SEQ ID NO: 39 - DNA, 28 bp, Artificial Sequence
  synthetic
gactctagatctgagattctcaggatct

SEQ ID NO: 40 - DNA, 25 bp, Artificial Sequence
  synthetic
tcctgagaatctcagatctagagtc

SEQ ID NO: 41 - DNA, 26 bp, Artificial Sequence
  synthetic
gactctagatctgagattctcaggat

SEQ ID NO: 42 - DNA, 23 bp, Artificial Sequence
  synthetic
ctgagaatctcagatctagagtc

SEQ ID NO: 43 - DNA, 24 bp, Artificial Sequence
  synthetic
gactctagatctgagattctcagg

SEQ ID NO: 44 - DNA, 21 bp, Artificial Sequence
  synthetic
gagaatctcagatctagagtc

SEQ ID NO: 45 - DNA, 22 bp, Artificial Sequence
  synthetic
gactctagatctgagattctca

SEQ ID NO: 46 - DNA, 12 bp, Artificial Sequence
  synthetic
ttttagtcaata

SEQ ID NO: 47 - DNA, 12 bp, Artificial Sequence
  synthetic
ttttagtcaatc

SEQ ID NO: 48 - DNA, 12 bp, Artificial Sequence
  synthetic
ttttagtcaatg

SEQ ID NO: 49 - DNA, 12 bp, Artificial Sequence
  synthetic
ttttagtcaatt

SEQ ID NO: 50 - DNA, 12 bp, Artificial Sequence
  synthetic
ttttagtcgata

SEQ ID NO: 51 - DNA, 12 bp, Artificial Sequence
  synthetic
ttttagtcgatc

SEQ ID NO: 52 - DNA, 12 bp, Artificial Sequence
  synthetic
ttttagtcgatg

SEQ ID NO: 53 - DNA, 12 bp, Artificial Sequence
  synthetic
ttttagtcgatt

SEQ ID NO: 54 - DNA, 24 bp, Artificial Sequence
  synthetic
agatcctgagaatctcagatctag

SEQ ID NO: 55 - DNA, 27 bp, Artificial Sequence
  synthetic
actctagatctgagattctcaggatct

SEQ ID NO: 56 - DNA, 18 bp, Artificial Sequence
  synthetic
caggggttactttggtgc

SEQ ID NO: 57 - DNA, 21 bp, Artificial Sequence
  synthetic
adtgcaccaaagtaacccctg

SEQ ID NO: 58 - DNA, 33 bp, Artificial Sequence
  synthetic
attcggcgagcatcggaaactggagagtcagat

SEQ ID NO: 59 - DNA, 29 bp, Artificial Sequence
  synthetic
gtcgtcggtaatcatgctaatcccgggat

SEQ ID NO: 60 - DNA, 29 bp, Artificial Sequence
  synthetic
atcccgggattagcatgattaccgacgac

SEQ ID NO: 61 - DNA, 17 bp, Artificial Sequence
  synthetic
attcggcgagcatcgga

SEQ ID NO: 62 - DNA, 17 bp, Artificial Sequence
  synthetic
gtcgtcggtaatcatgc

SEQ ID NO: 63 - DNA, 14 bp, Artificial Sequence
  synthetic
gatgtgggtttgca

SEQ ID NO: 64 - DNA, 36 bp, Artificial Sequence
  synthetic
aacccacatctacagaccctgaagaaacagtcnnnn

SEQ ID NO: 65 - DNA, 36 bp, Artificial Sequence
  synthetic
aacccacatctacagaccaagctgaaacagtcnnnn

SEQ ID NO: 66 - DNA, 18 bp, Artificial Sequence
  synthetic
cgcgagggagatcacttc

SEQ ID NO: 67 - DNA, 18 bp, Artificial Sequence
  synthetic
gactgaagtgatctccct

SEQ ID NO: 68 - DNA, 18 bp, Artificial Sequence
  synthetic
agtcgaagtgatctccct

SEQ ID NO: 69 - DNA, 13 bp, Artificial Sequence
  synthetic
ttcagggtctgta

SEQ ID NO: 70 - DNA, 24 bp, Artificial Sequence
  synthetic
acgagtacatcgaactacggctag

SEQ ID NO: 71 - DNA, 24 bp, Artificial Sequence
  synthetic
ggagttctgcatcacttgacgcta

SEQ ID NO: 72 - DNA, 24 bp, Artificial Sequence
  synthetic
tgtcggtgcgtcatttctaaggag

SEQ ID NO: 73 - DNA, 24 bp, Artificial Sequence
  synthetic
ggatccgaattcactcgggccaag

SEQ ID NO: 74 - DNA, 26 bp, Artificial Sequence
  synthetic
tactagccgtagttcgatgtactcgt

SEQ ID NO: 75 - DNA, 26 bp, Artificial Sequence
  synthetic
tatagcgtcaagtgatgcagaactcc

SEQ ID NO: 76 - DNA, 26 bp, Artificial Sequence
  synthetic
tactccttagaaatgacgcaccgaca

SEQ ID NO: 77 - DNA, 26 bp, Artificial Sequence
  synthetic
tacttggcccgagtgaattcggatcc

SEQ ID NO: 78 - DNA, 47 bp, Artificial Sequence
  synthetic
gtcgtcggtaatcatgcgatatcacgagtacatcgaactacggctag

SEQ ID NO: 79 - DNA, 47 bp, Artificial Sequence
  synthetic
gtcgtcggtaatcatgcgatatcggagttctgcatcacttgacgcta

SEQ ID NO: 80 - DNA, 47 bp, Artificial Sequence
  synthetic
gtcgtcggtaatcatgcgatatctgtcggtgcgtcatttctaaggag

SEQ ID NO: 81 - DNA, 47 bp, Artificial Sequence
  synthetic
gtcgtcggtaatcatgcgatatcggatccgaattcactcgggccaag

SEQ ID NO: 82 - DNA, 49 bp, Artificial Sequence
  synthetic
tactagccgtagttcgatgtactcgtgatatcgcatgattaccgacgac

SEQ ID NO: 83 - DNA, 49 bp, Artificial Sequence
  synthetic
tatagcgtcaagtgatgcagaactccgatatcgcatgattaccgacgac

SEQ ID NO: 84 - DNA, 49 bp, Artificial Sequence
  synthetic
tactccttagaaatgacgcaccgacagatatcgcatgattaccgacgac

SEQ ID NO: 85 - DNA, 49 bp, Artificial Sequence
  synthetic
tacttggcccgagtgaattcggatccgatatcgcatgattaccgacgac

SEQ ID NO: 86 - DNA, 17 bp, Artificial Sequence
  synthetic
gtcgtcggtaatcatgc

SEQ ID NO: 87 - DNA, 17 bp, Artificial Sequence
  synthetic
gtcgtcggtaatcatgc

SEQ ID NO: 88 - DNA, 17 bp, Artificial Sequence
  synthetic
gtcgtcggtaatcatgc

SEQ ID NO: 89 - DNA, 17 bp, Artificial Sequence
  synthetic
gtcgtcggtaatcatgc

SEQ ID NO: 90 - DNA, 17 bp, Artificial Sequence
  synthetic
gtcgtcggtaatcatgc

SEQ ID NO: 91 - DNA, 59 bp, Artificial Sequence
  synthetic
acgagtacatcgaactacggctagacccacatctacagaccctgaagaaacagtcnnnn

SEQ ID NO: 92 - DNA, 31 bp, Artificial Sequence
  synthetic
ggtctgtagatgtgggtgaagtgatctccct

SEQ ID NO: 93 - DNA, 18 bp, Artificial Sequence
  synthetic
cgcgagggagatcacttc

SEQ ID NO: 94 - DNA, 20 bp, Artificial Sequence
  synthetic
ctgagtccgatgctgttcaa

SEQ ID NO: 95 - DNA, 20 bp, Artificial Sequence
  synthetic
ttgaacagcatcggactcag

SEQ ID NO: 96 - DNA, 20 bp, Artificial Sequence
  synthetic
gcagcttgcagacccataat

SEQ ID NO: 97 - DNA, 20 bp, Artificial Sequence
  synthetic
cgctctaatctctgggcatc

SEQ ID NO: 98 - DNA, 20 bp, Artificial Sequence
  synthetic
gatgcccagagattagagcg

SEQ ID NO: 99 - DNA, 20 bp, Artificial Sequence
  synthetic
cctgattcagcgaaaccaat

SEQ ID NO: 100 - DNA, 20 bp, Artificial Sequence
  synthetic
gtgctattgctggcggtatt

SEQ ID NO: 101 - DNA, 20 bp, Artificial Sequence
  synthetic
aataccgccagcaatagcac

SEQ ID NO: 102 - DNA, 20 bp, Artificial Sequence
  synthetic
gaaatgccacaagcctcaat

SEQ ID NO: 103 - DNA, 20 bp, Artificial Sequence
  synthetic
tctttctcaatccccaatgc

SEQ ID NO: 104 - DNA, 20 bp, Artificial Sequence
  synthetic
cgattggggattgagaaaga

SEQ ID NO: 105 - DNA, 20 bp, Artificial Sequence
  synthetic
ttcccagcctcaatctcatc

SEQ ID NO: 106 - DNA, 20 bp, Artificial Sequence
  synthetic
cctgtgacgacaaatctgct

SEQ ID NO: 107 - DNA, 20 bp, Artificial Sequence
  synthetic
agcagatttgtcgtcacagg

SEQ ID NO: 108 - DNA, 20 bp, Artificial Sequence
  synthetic
ccagcagtccacttcgattt

SEQ ID NO: 109 - DNA, 44 bp, Artificial Sequence
  synthetic
acgagtacatcgaactacggctagttcccagcctcaatctcatc

SEQ ID NO: 110 - DNA, 44 bp, Artificial Sequence
  synthetic
ggagttctgcatcacttgacgctattcccagcctcaatctcatc

SEQ ID NO: 111 - DNA, 44 bp, Artificial Sequence
  synthetic
tgtcggtgcgtcatttctaaggagttcccagcctcaatctcatc

SEQ ID NO: 112 - DNA, 44 bp, Artificial Sequence
  synthetic
ggatccgaattcactcgggccaagttcccagcctcaatctcatc

SEQ ID NO: 113 - DNA, 26 bp, Artificial Sequence
  synthetic
gacctgagaatcacagacccgggatc

SEQ ID NO: 114 - DNA, 26 bp, Artificial Sequence
  synthetic
gatcccgggtctgtgattctcaggtc

SEQ ID NO: 115 - DNA, 21 bp, Artificial Sequence
  synthetic
attcggcgagcatcggaagta

SEQ ID NO: 116 - DNA, 18 bp, Artificial Sequence
  synthetic
ttccgatgctcgccgaat

(19) United States
(12) Patent Application Publication
(10) Pub. No.: US 2009/0068645 A1
(43) Pub. Date: Mar. 12, 2009

(54) Labeling and Sequencing of Nucleic Acids

(75) Inventor: Ross Sibson, Cheshire (GB)

Correspondence Address: CLARK & ELBING LLP, 101 FEDERAL STREET, BOSTON, MA, 02110, US

(73) Assignee: Interaseq Genetics Limited, Liverpool (GB)

(21) Appl. No.: 11/577,024

(22) PCT Filed: Oct. 11, 2005
(86) PCT No.: PCT/GB05/03921

§ 371 Date: Jan. 23, 2008

(30) Foreign Application Priority Data
Oct. 11, 2004 (GB) 0422551.2

(51) Int. Cl. C12Q 1/68

(52) U.S. Cl. 435/6

(57) ABSTRACT

The invention provides for methods of end labelling a ds DNA molecule with a bar-code as well as adaptor mediated methodology for creating single stranded overhanging ends, lengthening single stranded overhanging ends, controlled size reduction of labelled ds DNA molecules, and adaptor mediated sequencing. Also disclosed are methods for parallel sequencing of multiple ds DNA molecules in a mixture by visual means.

### BACKGROUND

Determining one or more nucleotides in one or a plurality of nucleic acids has become a major activity leading to useful and valuable understanding of biological systems. Methods of sequence determination are rate limiting for analysis and costly to implement.

Since the first reliable methods for determining nucleic acid sequences were reported in 1997 (Sanger, et al, DNA sequencing with chain-terminating inhibitors, Proc Natl Acad Sci USA (1977) 74(12) 5463-7, incorporated herein by reference for all purposes) there has been an exponential increase in their use. The methods have been constantly refined or new methods introduced with the aim of making sequence reading more efficient and effective in terms of ease of use, throughput, reliability and cost. Innovations that have been used in isolation or in combination include the use of automation, alternative chemistries, alternative labels especially fluorescent labels in place of radioactivity for automated reading, automating chemistry especially through the solid phase approaches, alternative separations especially capillary electrophoresis, mass spectroscopy and microfluidics. Multiplex assays have been used to increase throughput. New approaches include sequencing by hybridisation and arrays and these in turn have been further developed or refined.

It was realised at least as early as 1989 that single molecule approaches could offer the ultimate performance in terms of increased throughput and reduced costs. Biophysical methods have been developed including the analysis of nucleic acids through their interaction with a device like an atomic force microscope or on electrophoresis through a nanopore. Lasers and other tools like molecular tethers or lasers have been used to immobilise single molecules for analysis by enzymatic manipulation and subsequent recording of the reaction products. Effort has been put into improving the detection sensitivity through the recording devices and/or the labels. Enzymes have also been engineered to improve their performance in the analytical manipulations of the nucleic acids, especially where tolerance of artificial substrates was required. Commonly, analysis and detection have to be coupled in a single instrument thus limiting throughput to the rate limiting step.

A step that is common to most methods of determining nucleic acid sequence is recording the nucleotide at the end of a fragment. In order for nucleotide+1 sequences to be determined, nucleotides that are internal to the end are iteratively brought to the ends for the purpose of recording their identity. Either the action of a polymerase or an exonuclease can bring this about. Polymerases have the advantage that modified nucleotides can be incorporated during synthesis. These can control growth of the nucleotide strand in a template dependent fashion. In addition, the modified nucleotides can facilitate recording for example by being appropriately labelled. Exonucleolytic methods are the corollary of polymerase based methods in that the former remove nucleotides which have been optionally labelled. Recording occurs immediately prior or post removal. Biophysical methods move strands of nucleotides past a nucleotide recorder one nucleotide at a time or alternatively move the recorder along the strands of nucleotides.

Ligases have been used for labelling the sequences at the ends of fragments (see e.g., WO 94/01582 incorporated herein by reference for all purposes). Typically, a cohesive end is produced at the end and an adaptor molecule is then ligated to the cohesive end. Availability of a plurality of adaptor molecules each specific for a particular cohesive end allows the actual nucleotide sequence in the ends to be determined. Such methods have several advantages. More than one nucleotide at a time can be identified per end since each cohesive end is typically more than a single nucleotide in length. Exposure of the next nucleotide of interest is achieved through the action of a type11s restriction endonuclease whose site is placed in the adaptor used for detection to allow cutting of the fragment under investigation so that the required bases are left on the end (see e.g., WO 95/20053 incorporated herein by reference for all purposes). This process can therefore be highly processive in that cycles of cutting and ligation systematically move through the fragments of interest. Polymerase or exonucleolytic reactions are much more difficult to control requiring either precise reaction mixtures, manipulation of individual molecules, typically with a laser, or elaborately modified nucleotides which serve as chain terminators that upon chemical modification allow further chain elongation. Massively parallel sequencing has been achieved through cyclical cutting and ligation (see e.g., Brenner et al, Nature Biotechnology, Vol 18, 630-634, incorporated herein by reference for all purposes). This is an elaborate process whereby each fragment of interest in a mixture is first labelled with its own a unique hybridisable tag. A plurality of beads are used each of which has attached multiple copies of a unique oligonucleotide that is able to capture by hybridisation just one type of tag used to label the fragments. Each fragment can therefore be uniquely captured by a particular bead. Prior amplification of the fragments allows the beads to capture multiple instances of a given fragment thus facilitating detection. The cyclical cutting, ligation and detection process is then performed on the beads so that a unique nucleotide sequence is read from each. Positions of the beads have to be tracked between each round of analysis.

High throughput sequencing has also been performed with polymerase based sequencing. Nucleotide strands of interest are randomly fixed to a flat surface. Template directed synthesis is used to incorporate a single-nucleotide per immobilised strand. Elaborate modifications of the incorporated nucleotide are required to enable the process. One type of modification is required to prevent chain extension once the first nucleotide per strand has been incorporated. A label on the incorporated nucleotide is also required so that it can be identified. This label has to be sufficiently bright that even the single-molecules immobilised can be detected. Chemical modification of the incorporated nucleotide following detection allows the next nucleotide to be incorporated for further rounds of detection. Multiple rounds allow large amounts of sequence to be determined but this requires the positions of the single molecules each to be tracked. When analysing complex nucleic acids it is beneficial to be able to determine long sequences. This is because short sequences occur multiple times in a complex nucleic acid and if determined as short reads by the process they cannot easily be distinguished. A special demand is placed on the process just described because each single-molecule that fails to reach a point in the process where it has given a sufficiently long sequence has to be discarded from the analysis. Reasons for the failure can include modified nucleotides that are not 100% pure, incorporation efficiencies of less than 100% and modification efficiencies of less than 100%. Determining sequence from immobilised molecules means that rounds of detection cannot occur per molecule until chemical modification is complete and therefore the duration of the chemistry cycles ultimately determines the rate of production of sequence per molecule. There is a similar problem with MPSS described above except that the rate of MPSS is determined by the rates of cutting and ligation on the solid support, beads in this case. MPSS also suffers from the disadvantage that neither the rates of ligation nor restriction are 100% so that on a particular bead the molecules tend to get out of phase with respect to the cycle of the process in which they are supposed to be present. Ultimately this limits the number of cycles from which reliable data can be obtained and thus the amount of sequence per fragment that can be obtained.

The invention seeks to overcome problems of the prior art, and provides a method whereby nucleic acids can be labelled according to their sequence identity. New and/or known nucleic acids can thus be compared and sequenced in a massively parallel format.

### SUMMARY OF THE INVENTION

In one aspect, the present invention provides a method of differentially labelling one end of a double stranded (ds) DNA molecule on the basis of its nucleic acid sequence. This method comprises the following steps:

(a) providing said ds DNA molecule in linear form with at least one single stranded overhanging end;
(b) incubating, under conditions suitable to allow for DNA ligation, said ds DNA molecule having at least one single stranded overhanging end with a pool of different indexing molecules, the different indexing molecules of the pool having complementary single stranded ends for annealing to the at least one overhanging end of the ds DNA molecule, said different indexing molecules being labelled and distinguishable from one another, to produce a linear ligation product having indexing molecules at each end thereof;
(c) circularising the linear ligation product of step (b) by incubating under conditions suitable to allow for DNA ligation;
(d) linearising the circular product of step (c) by cleavage with a restriction enzyme having a cleavage site that is physically displaced from its recognition site, said recognition site being present in a portion of the circular product which is not derived from the original ds DNA molecule to be labelled, and said cleavage site being located within a portion of said circular product which is derived from the original ds DNA molecule to be labelled, such that a linear DNA molecule is produced having single stranded overhanging ends with nucleotide sequences characteristic of the ds DNA molecule; and, optionally
(e) repeating steps (b) to (d) one or more times.

In one embodiment, the above method can be used to differentially label two or more ds DNA molecules in a mixture containing a plurality of ds DNA molecules said plurality of ds DNA molecules of the mixture having different nucleic acid sequences. Such a method has the advantage of allowing for individual DNA molecules within the mixture to be separately identified and categorised according to their nucleic acid sequence. Having identified the individual DNA molecules, further characterisations such as sequence analysis, can be performed.

In some embodiments both ends of the ds DNA molecule are provided with single stranded overhanging ends. In other embodiments one end of the ds DNA molecule has a single stranded overhanging end, and the opposite end is blunt ended.

The pool of indexing molecules may comprise indexing molecules with all possible complementary single stranded ends of a predetermined length for annealing and ligating to all possible single stranded overhanging ends of the ds DNA molecule, or may have a selection of indexing molecules representing a sub-set of the full group.

In one embodiment, ds DNA molecules labelled according to the methods of the invention can be subjected to adaptored sequencing of the end distal to the labelled end. Such adaptored sequencing can be performed directly on the labelled molecules, as discussed further below, or can be applied to fragments of the labelled molecule. Fragments may be generated by one or more rounds of adaptor-mediated controlled size reduction, as discussed further below, or by random fragmentation and restriction digestion followed by size purification so that fragments of known sizes and end sequences are used.

Accordingly, in a further aspect, the invention provides a method for controlled size reduction of a ds DNA molecule labelled according to the methods disclosed herein. The method of controlled size reduction comprises the steps of:

(a) incubating, under conditions suitable to allow for DNA ligation, said labelled ds DNA molecule with a pool of different adaptor molecules, the different adaptor molecules of the pool having:
(i) complementary single stranded ends for annealing to the overhanging end of the ds DNA molecule distal to the indexing molecule derived label thereof; and
(ii) a recognition site for a restriction enzyme having a cleavage site that is physically displaced from its recognition site in order to provide a labelled ds DNA molecule/adaptor ligation product wherein the cleavage site for the restriction enzyme of step (a)(ii) is located within a portion of the labelled ds DNA molecule/adaptor ligation product derived from the labelled ds DNA molecule;
(b) cleaving said labelled ds DNA molecule/adaptor ligation product with said restriction enzyme of step (a)(ii) in order to remove the adaptor molecule and to reduce the length of the labelled ds DNA molecule by a controlled number of nucleic acid bases; and, optionally
(c) repeating steps (a) and (b) one or more times to achieve further controlled size reductions.

In one embodiment, the labelled ds DNA subject to size reduction is one of a plurality of labelled DNA molecules in a mixture, said plurality of labelled ds DNA molecules of the mixture having different nucleic acid sequences, wherein the length of two or more of the plurality of labelled ds DNA molecules is reduced by a controlled number of bases.

The method of controlled size reduction as outlined above can be used to generate labelled fragments of differing lengths. Sequence information for these labelled individual fragments can then be determined by exposure of the fragments to sequence specific adapters.

Accordingly, in yet a further aspect, the invention provides a method of determining one or more bases of a ds DNA molecule labelled as described herein. This method comprises the steps of:

(a) incubating, under conditions suitable to allow for DNA ligation, the labelled ds DNA molecule with a pool of different sequencing adaptor molecules, the different sequencing adaptor molecules of the pool having complementary single stranded ends for annealing to the overhanging end of the ds DNA molecule distal to the indexing molecule derived label thereof, each of the different sequencing adaptor molecules being differentially labelled, in order to provide a labelled ds DNA molecule/sequencing adaptor ligation product; and
(b) detecting the sequencing adaptor ligated to the labelled ds DNA molecule in step (a) and determining one or more bases of the ds DNA molecule on the basis of the sequencing adaptor detected.

In one embodiment of the invention, the labelled ds DNA molecule subject to incubation with different sequence adapter molecules has been reduced in length in a controlled manner according to the size reduction methods outlined herein.

In yet a further embodiment, the methods of determining one or more bases of a ds DNA molecule are carried out on a ds DNA molecule which is one of a plurality of labelled DNA molecules in a mixture, said plurality of labelled ds DNA molecules of the mixture having different nucleic acid sequences, and wherein one or more nucleic acid bases are determined for two or more of said plurality of labelled ds DNA molecules.

In some embodiments the pool of sequencing adaptor molecules will contain molecules with all possible complementary single stranded ends for annealing to the overhanging ends of the ds DNA molecule, for example, when no sequence information is known. In other embodiments only those complementary single stranded ends as are required for the application of interest are used. For example, if the method is used to determine or distinguish known polymorphisms then only as many sequence adaptor molecules as are required for annealing to the known polymorphic sequences are required. Similarly, if the method is to be used to detect features such as allelic imbalance, then, again, only as many sequencing adaptors as are required to detect the feature are needed.

In another aspect, the invention provides a method for determining at least a partial nucleic acid sequence of two or more ds DNA molecules in a mixed population of ds DNA molecules having different nucleotide sequences. The method according to this aspect of the invention comprises the following steps:

(a) differentially labelling one end of said two or more ds DNA molecules in said mixed population according to a method as described herein;
(b) conducting controlled size reduction of the differentially labelled ds DNA molecules in the mixed population, according to the method outlined herein; and
c) determining, according to the adapted sequencing method outlined herein, one or more nucleic acid bases for two or more of the labelled ds DNA molecules in a sample having undergone controlled size reduction.

In one embodiment, one or more nucleic acid bases are determined for the two or more labelled ds DNA molecules in at least two samples, each sample having undergone controlled size reduction of the labelled ds DNA molecules therein to a different extent. In some cases, the amount of controlled size reduction is controlled by subjecting the population of labelled ds DNA molecules to multiple rounds of controlled size reduction. Accordingly, in some embodiments, multiple rounds of controlled size reduction are carried out, and the determining of one or more nucleic acid bases for two or more of the labelled ds DNA molecules is carried out on samples having undergone each round of controlled size reduction.

In some embodiments, the mixed population of differentially labelled ds DNA molecules is separated into individual pools for controlled size reduction, each pool being subject to controlled size reduction to a different extent. In other embodiments, multiple rounds of controlled size reduction are carried out on a single pool and that pool is then sampled following at least one round of controlled size reduction for determining one or more nucleic acid bases for two or more of the labelled ds DNA molecules therein. In some embodiments, the pool is sampled after each round of controlled size reduction for the determination of one or more nucleic acid bases for two or more of the labelled ds DNA molecules therein.

Methods of the invention for determining at least a partial nucleic acid sequence of two or more ds DNA molecules in a mixed population of ds DNA molecules having different nucleotide sequences as described herein provide for rapid easy analysis of nucleic acid sequence of a large number of different DNA molecules in a sample at the same time, without the need for separation for DNA molecules according to their sequence. Determination of the sequence can be by purely visual means or may involve the use of digital image processing means together with appropriate algorithms in a computerised system.

In a further aspect, the invention provides methods of providing a single stranded overhanging end on a double stranded (ds) DNA molecule comprising engineering a nick in one strand of said ds DNA molecule, and dissociating away from said ds DNA molecule a single stranded fragment from said nicked strand, said fragment extending from the first end to the site of the nick.

In preferred embodiments, the nick may be engineered by ligating a double stranded adaptor molecule to the ds DNA molecule. For example, a modified adaptor may be used in which one strand only becomes covalently attached to the ds DNA molecule upon ligation, thereby creating a nick in the other strand. Alternatively, the adaptor molecule may be designed such that upon ligation a nicking endonuclease recognition site is located within the adaptor and its cognate nicking site is located within the original ds DNA molecule. Incubation of the ligation product with the nicking endonuclease results in a nick in one strand.

In yet a further aspect, the invention provides a process for lengthening a single stranded overhanging end on a double stranded (ds) DNA molecule. The method according to this aspect of the invention comprises:

a) ligating to said single strand overhang a lengthening adaptor molecule, said lengthening adaptor molecule having a recognition site for a nicking endonuclease, or being designed to create a recognition site for a nicking endonuclease on ligation to said ds DNA molecule, but lacking a cognate nick site for said nicking endonuclease,
wherein the nick site for said nicking endonuclease following ligation to a ds DNA molecule is located within a portion of the ligation product derived from the ds DNA molecule;
b) incubating the ligation product of step a) with a nicking endonuclease that recognises said nicking endonuclease recognition site in order to nick a first strand of the ds DNA molecule but not a second strand thereof; and
c) cleaving the lengthening adaptor in a predetermined position so that the ds DNA molecule remains ligated to a nucleotide sequence derived from the lengthening adaptor on the second strand, and so that the nucleotide sequence between the point of cleavage and the nick site on the first strand dissociates to leave a lengthened single stranded overhang.

In some embodiments, cleavage of the lengthening adaptor occurs before incubation with the nicking endonuclease, and in other embodiments cleavage of the lengthening adaptor occurs after incubation with the nicking endonuclease. Cleavage may be brought about by the action of an enzyme or any other process that achieves fragmentation in the desired predetermined location. Thus, the lengthening adaptor may be designed to include a recognition site for a restriction endonuclease to allow cleavage by that enzyme at a predetermined site, and cleavage of the lengthening adaptor may then be brought about by the action of that restriction enzyme at that site. In other embodiments, the lengthening adaptor is synthesised to contain dUTP at the predetermined cleavage site, and cleavage may then be brought about by the action of uracil-DNA glycosylase (UNG) at that site

The above defined process for lengthening a single stranded overhanging end on a double stranded (ds) DNA molecule is useful, for example, in creating overhanging ends of the ds DNA molecule which can be ligated with high fidelity to molecules having complementary overhanging ends. In particular therefore, one embodiment of the invention utilises the above lengthening process in creating lengthened single stranded overhanging ends of the ds DNA molecule for annealing and ligation to indexing molecules, adaptors or sequencing adaptors. Optionally therefore, the single stranded overhanging ends of the ds DNA molecules are lengthened according to the above process and then subject to any one or combination of the methods of differential labelling, size reduction, and sequence determination as outlined herein.

### BRIEF DESCRIPTION OF THE FIGURES

FIG. 1 shows the recognition and cleavage sites of an example of a type II s restriction endonuclease, namely BpmI, and the results of cleavage using that enzyme.

FIG. 2 shows the restriction site and cleavage site of an example interrupted palindrome restriction enzyme, namely Bgl I and the result of cleavage using that enzyme.

FIG. 3 shows an example of single stranded end lengthening according to a method of the invention using a lengthening adapter to provide a nine base pair 3′ overhang.

FIGS. 4A and 4B show the bar-code end labelling method of the invention.

FIG. 5A shows the method of controlled size reduction of the invention practised on a bar-coded end labelled double strength DNA molecule.

FIG. 5B shows the principal of controlled size reduction in more detail with reference to Bpm I as an example enzyme to be used.

FIG. 6 shows parallel sequencing of three different ds DNA molecules each having a different bar-code end label, and each having undergone ordered shortening according to the methods of the invention. One of the three bar-coded DNA molecules is highlighted and the derivation of its sequence is shown.

FIG. 7 Parts A to D depict the molecular indexing of DNA and sequence (Midas) methodology of the invention.

FIG. 8 shows ligase dependence of indexing of HinfI-cut phiX174 DNA. Electropherograms of indexing reactions performed under different conditions are provided. A) Pfu DNA ligase, 37° C., magnesium. B) Pfu DNA ligase, 37° C., manganese. C) Taq ligase, 45° C.

FIG. 9 show electropherograms showing the effect on product yields of varying the ligase and indexer amounts. A) Pfu ligase. B) Taq ligase.

FIG. 10 shows electropherograms showing indexing at an originally blunt end.

FIG. 11 shows a comparison of the frequencies in human RefSeq Build 35.1 for BglII or SalI selected for 2 further bases shown, plus an additional 10 adjacent bases definable by indexing at each site (see Example 6).

FIG. 12 shows a procedure for the isolation of indexed short genomic sequences: (1) HinfI digest of human genomic DNA. (2) Ligation of blocker and Initial Indexer. (3) N.BstNBI digest followed by XbaeI digest. (4) Ligation of biotinylated indexes. (5) BpnI digest. (6) Ligation of PCR indexer.

FIG. 13 shows the circularization and re-cutting of ΦX174 fragments indexed at both ends.

FIG. 14 shows a strategy for making bar coded indexers.

FIG. 15 shows a Catherine wheel labelling system for short branched indexers.

FIG. 16 shows an agarose gel of labelled M13 fragments hybridised to single-stranded M13 to form a Catherine wheel probe.

FIG. 17 shows a photomicrograph of concentric drying rings with DNA labelled with YOYO-1 post spreading, showing DNA molecules stretched by the meniscus.

FIG. 18 shows single molecules of phage Lambda DNA.

FIG. 19 shows the absence of DNA stretching for Alexofluor labelled DNA.

FIG. 20 shows halos of AlexaFluor-labelled DNA spread in CHAPS buffer, before drying.

FIG. 21A and A shows higher magnification of individual halos of AlexaFluor-labelled DNA spread in CHAPS buffer before drying.

### DETAILED DESCRIPTION OF THE INVENTION

The following specification sets out in more detail methods for labelling one end of a double stranded (ds) DNA molecule, methods for reducing the length of so labelled ds DNA molecules in a controlled manner, and methods for determining one or more nucleic acid bases of so labelled ds DNA molecules. Also described are methods in which a mixed population of ds DNA molecules can be simultaneously differentially labelled at one end, and subsequently sequenced. In large part, the methods of the current invention rely upon annealing and ligation of various molecules to single stranded overhanging ends of ds DNA molecules of interest. The invention further provides methods for the lengthening of overhanging ends which allows for the use of, for example, thermostable DNA ligase enzymes having improved fidelity.

In various aspects and embodiments of the invention, adaptor-like molecules are ligated to the ds DNA molecule or molecules of interest. For the purposes of clarity only in this document, the terminology used to describe adaptor-like molecules depends upon the function of the adaptor-like molecule being described. For example, in descriptions of methods relating to end labelling of ds DNA molecules, adaptor-like molecules are referred to as “indexing molecules”. In descriptions of methods for controlled size reduction the term “adaptors” is used, and in descriptions of methods for sequencing and nucleotide base determination, the term “sequencing adaptors” is used. In descriptions of methods for lengthening single stranded overhanging ends, the term “lengthening adaptors” is used. No structural difference is intended to be implied merely by the differing terminology, and as will be readily apparent to the skilled reader, in some circumstances adaptor like molecules having structural characteristics in common may be employed for more than one of the above noted purposes. The generic terms “adaptor-like molecules” and “adaptor-mediated” are intended to refer to any of the above functionally defined species or their uses.

In various aspects and embodiments certain enzymes such as (but not limited to) type II, type II s, nicking and interrupted palindrome restriction enzymes, and thermostable and other DNA ligase enzymes, are employed. Unless otherwise stated, conditions for such enzymes usage are those conditions as set out by the manufacturers instructions, or as can readily be determined by the skilled person.

The invention provides for the differential labelling (also referred to as “tagging” or “indexing”) of ds DNA molecules on the basis of their nucleotide sequence. The labelling method of the invention involves one or more rounds of addition of indexing molecules to each end of the ds DNA molecule which is provided in linear form with overhanging single stranded ends. Following addition of the indexing molecules, the so adaptored molecule is circularised in order to bring the two indexing molecules together, and is subsequently linearised in such a way as to provide a linear molecule having single stranded overhanging ends on both ends, and having the two indexing molecules located together, proximal to one end thereof. The single stranded overhanging ends have sequences derived from the original ds DNA molecule. Subsequent rounds of ligation to indexing molecules, circularisation and linearization can add further indexing molecules at or close to one end of the ds DNA molecule.

Specificity of labelling is brought about by recognition of the single stranded overhanging ends of the ds DNA molecule. According to the method of the invention, at each round (although not, in some embodiments, the first round) the ds DNA is provided with single stranded overhanging ends having a sequence derived from, and therefore characteristic of the ds DNA molecule. Ligation is then effected with a pool of different indexing molecules. The different indexing molecules of the pool have different single stranded overhanging ends for annealing to the ds DNA overhanging ends, and are differentially labelled in such a way as to be recognisable depending on the sequence of their single stranded overhangs. Ligation of indexing molecules of the pool therefore results in selection of differentially labelled indexing molecules for incorporation into the ds DNA molecule, on the basis of the sequence of that ds DNA molecule.

As noted, a single round of the end labelling method of the invention results in two indexing molecules being incorporated into the ds DNA molecule proximal to one end thereof. Depending on the sequences of the single stranded overhanging ends targeted for ligation with the pool of indexing molecules, the labels on the two indexing molecules may be the same or different. Subsequent rounds of the end labelling method result in the incorporation of further indexing molecules chosen on the basis of the nucleic acid base sequences exposed during the linearization process (described in further detail below). In this way, the indexing molecules together generate a complex label similar in nature to a bar-code comprising each of the labels present on the particular indexing molecules that have been incorporated. A particular sequence of labels in a bar-code is indicative of the order of indexing molecules incorporated into the ds DNA molecule, and is, in turn, therefore indicative of at least part of the sequence of the ds DNA molecule. The bar-code label can be identified by visual means or by other pattern recognition means discussed in further detail below.

Providing ds DNA molecules with a bar-code label at one end thereof allows for the identification of a molecule's sequence or identity. Moreover, different ds DNA molecules in a population can be labelled simultaneously according to the methods of the invention and can subsequently be recognized and distinguished, using the bar-code, on the basis of their different nucleic acid sequence. In combination with other methods of the invention, bar-coding allows for rapid and simultaneous visual sequencing of multiple different ds DNA molecules in a single mixture.

It is common practice to increase the efficiency of sequencing chemistries through the use of multiplexing. In general, DNAs from different samples are each given a different distinguishing feature. Improvements in efficiency result because the different DNAs can then be pooled for further processing yet can be individually identified through their distinguishing features during further analysis. An early example was the use of different oligonucleotide sequences added as a tag to different DNAs and the use of hybridisation based approaches to separately recognise the individual tags (see Church G M and Kieffer-Higgins, Science. (1988) 240 p185-8 incorporated herein by reference for all purposes). It will be realised that the approach described herein can similarly be multiplexed. At any point the adapters that are to be retained on the target can include a recognisable tag that encodes their origin. Once the tags have been added to the targets the targets can be pooled and distinguished during further analysis. It is best to add such tags as early as possible (during the first round of ligation) so that pooling can be similarly early and greatest benefit in terms of reduced sample processing will be achieved. A 4 position 4 colour code for example produces 256 different tags allowing DNAs from 256 individuals to be separately identified following pooling.

In addition, and because of the specific incorporation of indexing molecules on the basis of complementarity of their overhanging ends to overhanging ends in the ds DNA molecule, labelling one end of the ds DNA molecule according to the methods of the invention also provides sequence information about the ds DNA molecule. It will be readily apparent to the skilled person, on appreciating the mechanism underlying the labelling method, that labelling in this way can be used to categorise DNA molecules on the basis of a limited amount of nucleic acid sequence, and can be used independently of other sequencing methods to provide information concerning the base sequence at certain positions in the ds DNA molecule.

As a preliminary measure for labelling of one end of a ds DNA molecule, the ds DNA molecule to be labelled is provided with at least one single stranded overhanging end. This can be achieved by any suitable means. In some embodiments, overhanging ends are generated by digestion of a DNA sample, from any source, with a type II restriction endonuclease, for example, such as DpnII. Other restriction enzymes will be known to the skilled person, and the only requirement of the restriction enzyme is that it cleaves the DNA in the sample in such a way as to leave single stranded overhanging ends. Single stranded overhanging ends can be on either strand of the ds DNA (i.e., can be 5′ overhangs or 3′ overhangs). Preparation of a ds DNA molecule with overhanging ends in this way results in the overhanging ends having a sequence characteristic of the cleavage site of the restriction endonuclease used, and also characteristic of the sequence of the ds DNA molecule.

Digestion of a DNA sample with some restriction enzymes will result in single stranded overhanging ends being identical for each DNA fragment produced, whereas other restriction enzymes will provide single stranded overhanging ends with greater degeneracy from one fragment to another, as a result of degeneracy within the particular enzymes' cleavage site. Subsequently, during the first round of addition of indexing molecules, fragments of ds DNA produced using restriction enzymes with fixed cleavage site sequences will result in all fragments containing the same indexing molecules after one round. Labelling ds DNA molecules cleaved with restriction enzymes having degenerate cleavage sites can result in selection of different indexing molecules, even during the first round of ligation, thereby resulting in the provision of sequence information and categorisation of ds DNA fragments produced by digestion. Two or more different restriction enzymes can be used simultaneously or in turn to generate a more complex pattern of single stranded overhanging ends on each ds DNA fragment.

Alternative means of providing single stranded overhanging ends on ds DNA molecules include ligation of adaptor like molecules to each end. In some circumstances this requires blunt ended ligation of adaptors to DNA samples without single stranded overhanging ends, and this can be achieved using methodology well known to those skilled in the art. Generation of single stranded overhanging ends in this way provides single stranded overhanging ends which are not necessarily characteristic, in sequence, of the sample ds DNA. However, for the first round of incorporation of indexing molecules this may not be important in some circumstances. In such circumstances, and because of the generation of single stranded overhanging ends with sequences characteristic of the ds DNA molecule, as described in greater detail below, further rounds of incorporation of indexing molecules can provide for specificity in the bar-code label.

An alternative means for providing ds DNA molecules with single stranded overhanging ends is by using the polymerase chain reaction or other amplification reactions to provide the ds DNA molecule. Primers used in, for example, PCR reactions, can be supplied with restriction endonuclease recognition sites, and post amplification the products may be cleaved with the appropriate restriction endonuclease to produce the single stranded overhanging ends.

A further means for providing ds DNA molecules with single stranded overhanging ends is the use of exonuclease enzymes, for example, the exonuclease activity associated with, for example, T4 DNA polymerase. As will readily be appreciated by the skilled person, the extent of exonuclease activity (i.e., the stopping point), can be defined e.g. by nucleotide modifications, for example thionucleotides, or by limiting the supply of nucleotides required by the polymerase in order to limit exonuclease/polymerase cycling.

Single stranded overhanging ends may also be provided by engineering into the ds DNA molecule a nick in one strand proximal to one end, and causing the dissociation of the resultant single stranded oligonucleotide fragment. The nick may conveniently be produced by any means known to the skilled person. In some embodiments the nick is engineered by ligating an adaptor molecule to one end of the ds DNA molecule. The adaptor may be modified by any means known to the skilled person such that on ligation only one strand of the adaptor becomes covalently linked to the ds DNA molecule. For example, the 5′ ends of the adaptor may be hydroxylated e.g., by the use of DNA phosphatase enzymes. Alternative blocking mechanisms will also be apparent, and may serve to prevent covalent linkage at either the 5′ or 3′ end of one strand of the adaptor. The result of the ligation reaction is a ds DNA molecule having a nick in one strand proximal to one end. A single stranded overhang may then be provided by causing the dissociation of the short single stranded oligonucleotide fragment from the nicked strand. The short single stranded oligonucleotide fragment extends from the end of the ligation product proximal to the nick to the nick site itself. Dissociation may be effected by providing appropriate temperature or solution conditions as will be apparent to the skilled person.

In other embodiments, the adaptor molecule will covalently link on ligation to the ds DNA molecules on both strands. The adaptor molecules can be provided with a recognition site for a nicking endonuclease enzyme, and may be designed such that a cognate nicking site is located in the ds DNA molecule. Following ligation, incubation with the nicking endonuclease will result in a nick in one strand of the ds DNA/adaptor molecule ligation product and, again, a resultant short single stranded oligonucleotide can be dissociated away to provide for a single stranded overhanging end.

Methods of the invention for providing overhanging ends by engineering a nick into one strand are advantageous as they allow for the production of overhanging ends on either the 5′ or 3′ of either (or both) strands. Moreover, suitable design of adaptor molecules allows for the engineering of overhanging ends of desired sequence or length.

Other means (e.g., physical means such as shearing), of providing single stranded overhanging ends will occur to the skilled person and may be used whether or not they provide for single stranded overhanging ends that are characteristic in sequence of the ds DNA molecule itself.

Thus, single stranded overhanging ends on the ds DNA molecules may be as short as a single nucleotide base, but in other circumstances longer overhangs can be used. Single stranded overhangs may be engineered or lengthened according to the methods of the invention in order to provide for single stranded overhanging ends of, typically, 4 or more bases in length, preferably 5 or more, e.g. 6 to 18, preferably 6, 7, 8, 9, 10, 11, 12, 13, 14, 15, 16, 17 or 18 bases in length. Such lengthened single stranded overhanging ends can subsequently be used in ligation reactions using, for example, thermostable DNA ligase enzymes having enhanced fidelity over DNA ligase enzymes such as T4 DNA ligase.

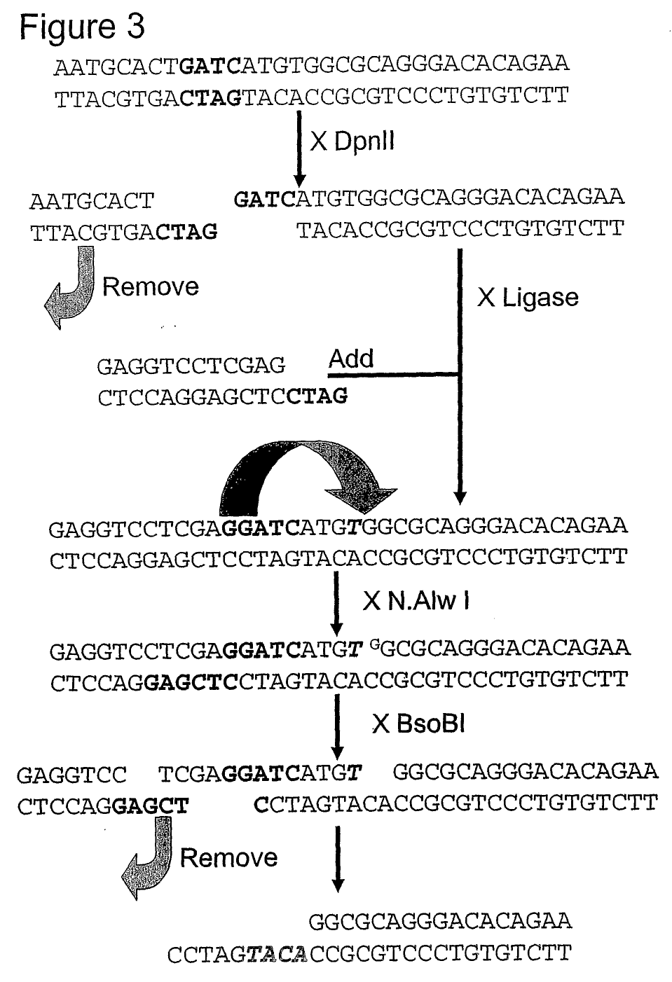

Lengthening of single stranded ends may proceed in the manner depicted in FIG. 3. Thus, a double stranded DNA molecule having a single stranded overhanging end (e.g. produced by the activity of an enzyme such as Dpn II) is ligated to a lengthening adapter having a complementary cohesive end. The lengthening adapter may include a recognition site for nicking restriction under nuclease such as N.Alw I, or may be designed such that on ligation to the end to be lengthened creates a recognition site for such an enzyme. The lengthening adapter is designed such that the nicking site for the nicking restriction under nuclease is located not within the lengthening adapter itself, but within a part of the resulting ligation product that is derived from the initial ds DNA molecule whose single stranded end it is desired to lengthen. Following incubation with the nicking endonuclease, a nick is therefore created in one strand of the original ds DNA molecule to be lengthened. Any known nicking endonuclease may be employed including engineered nicking endonucleases, for example fusions of known enzymes.

Following nicking, the lengthening adapter portion of the ligation product is cleaved at a predetermined point, for example by the example of a restriction endonuclease such as BsoB I in order to leave a short single stranded oligonucleotide which is then dissociated away from the ds DNA molecule having the end to be lengthened. As shown in FIG. 3, the result is a long single strand overhanging end, in the case shown in FIG. 3, that end being provided on the three prime end of one strand.

The ds DNA molecule to be labelled may be obtained from any source. In certain embodiments, genomic DNA can be isolated by methodology known to the skilled person and may be cleaved into suitable sized fragments by the use of restriction endonucleases (which, as described above, can provide for the necessary single stranded overhanging ends). In other embodiments, DNA can be provided using the polymerase chain reaction or other amplification reactions such as the ligase chain reaction. The ds DNA can be isolated from a natural source, or manufactured in vitro and can be genomic DNA or, for example, cDNA. The ds DNA molecules to be labelled according to their sequence can be in a homogenous solution in which all DNA molecules have the same, or substantially the same, nucleic acid sequence, or may be part of a heterogenous mixture of ds DNA molecules having differing nucleic acid sequences.

Once ds DNA molecules are provided having single stranded overhanging ends, whether lengthened or not, those ds DNA molecules are then ligated to indexing molecules. Indexing molecules are characterised by being DNA molecules having single stranded ends (complementary single stranded ends) suitable for annealing to the single stranded overhanging ends of the ds DNA molecule.

The indexing molecules are provided as a pool of different indexing molecules which differ in the sequence of their complementary single stranded ends such that at least a subset of all possible sequences complementary to the single stranded overhanging ends of the ds DNA molecule to be labelled are present. Indexing molecules with different single stranded end sequences are differentially labelled and can therefore be distinguished from each other. In the first round of addition of indexing molecules, in some circumstances, the sequences of the single stranded overhanging ends of the ds DNA molecule to be labelled will be known, and the sequences of the complementary single stranded ends of the indexing molecules of the pool can be chosen accordingly. In subsequent rounds of addition of indexing molecules the sequence of the single stranded overhanging ends of the ds DNA molecule are characteristic of the sequence of the molecule being labelled, and may, therefore, be unpredictable (although in some embodiments the sequence can be known). Where the sequence is unpredictable, if it is desired to ensure that substantially all ds DNA molecules are labelled it is necessary to include, within the pool, indexing molecules having all possible sequences in their complementary single stranded ends. In some circumstances it is sufficient only to label some ds DNA molecule species in a mixture and, in such cases, the skilled person is able to determine an appropriate subset combination of indexing molecule complementary single stranded end sequences for inclusion in the pool.

The end labelling method of the invention involves, initially, ligating indexing molecules to each end of the linear ds DNA molecule, followed by a step of circularising the linear molecule, and subsequently linearising the molecule again in such a way that each of the two indexing molecules remain together in the linear DNA molecule at or proximal to one end thereof. This step of linearization is achieved by using a restriction enzyme having a cleavage site that is physically displaced from its recognition site (referred to herein as a displaced cleavage restriction enzyme or endonuclease), such as a type IIs restriction enzyme or an interrupted palindrome restriction enzyme. One of the indexing molecules ligated to the linear DNA in the first step of each round of indexing provides the recognition site for the displaced cleavage restriction enzyme. The cleavage site of the enzyme is located within a portion of the molecule which is derived from the original ds DNA molecule to be labelled (i.e., not within the indexing molecules incorporated into the ds DNA molecule).

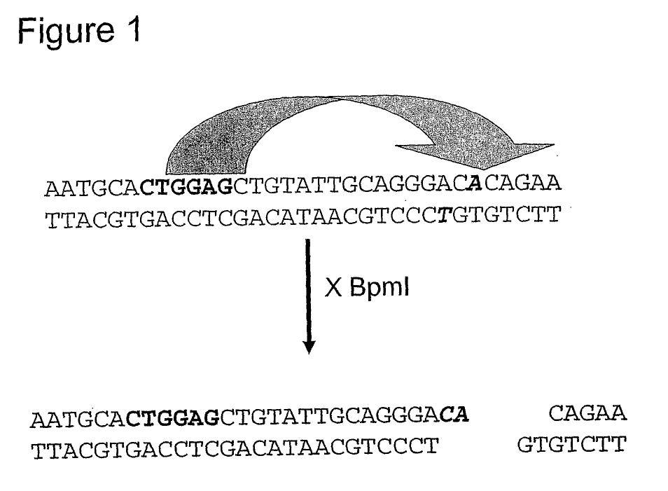

Suitable displaced cleavage restriction enzymes will be apparent to the skilled person, and are commercially available. By way of example, type IIs restriction enzymes are characterised in that the enzymes cut outside of their recognition site so that, in general, cleavage can result in any combination of bases in single stranded overhanging ends as a result. FIG. 1 shows how a type IIs restriction endonuclease produces fragments whose ends comprise a short sequence signature characteristic of the ends so produced. The cleavage site of a type IIs restriction enzyme is located a fixed distance away from the recognition site and as a result cleavage with such an enzyme always leaves a characteristic combination of bases for any given fragment being cleaved. By way of example in FIG. 1, the enzyme Bpm I is shown. Bpm I is characterised in having a cleavage site which is 16 bases (shown in bold and italic in the Figure) from its recognition site (which is shown in bold non-italic letters) in the top strand, and 14 bases (again shown in bold and italic) from its recognition site in the bottom strand. This leaves a two base pair 3′ single stranded overhanging end which can have any of 16(4²) possible DNA sequences dependent on the sequence of the DNA molecule cleaved. Not all type IIs restriction enzymes leave a single stranded overhanging end, and those enzymes which do not cannot be used in the methods of the invention without further modification of the resulting ends. Nucleases which can be employed in this process include restriction endonucleases, the cleavage sites of which are asymmetrically spaced across the two strands of a double stranded substrate, and the specificity of which is not affected by the nature of the bases adjacent to a cleavage site. Many enzymes other than Bpm I are available and will be apparent to the skilled person, exhibiting a wide range of specificities, and are commercially available (for a review see Roberts, R J et al, Nucl Acids Res 31 (2003) page 418-420 incorporated herein by reference for all purposes). Exemplary restriction endonucleases in this class are listed in Table 1.

TABLE 1

| | |
|---|---|
| Enzymes | Recognition Sequence |

Enzymes that recognise interrupted palindromes

| | |
|---|---|
| Ahd I | GACNNN/NNGTC |
| Ale I | CACNN/NNGTG |
| Alf I | (10/12)GCANNNNNNTGC(12/10) |
| AlwN I | CAGNNN/CTG |
| ApaB I | GCANNNNN/TGC |
| ApeK I | G/GWCC |
| Ava II | G/GWCC |
| Bgl I | GCCNNNN/NGGC |
| Blp I | GC/TNAGC |
| Bpl I | (8/13)GAGNNNNNCTC(13/8) |
| BsaB I | GATNN/NNATC |
| BsaJ I | C/CNNGG |
| BsaW I | W/CCGGW |
| BsiHKA I | GWGCW/C |
| Bsl I | CCNNNNN/NNGG |
| BssK I | /CCNGG |
| BstAP I | GCANNNN/NTGC |
| BstE II | G/GTNACC |
| BstN I | CC/WGG |
| BstX I | CCANNNNN/NTGG |
| Bsu36 I | CC/TNAGG |
| BthC I | GCNG/C |
| Cac8 I | GCN/NGC |
| Dde I | C/TNAG |
| Dra III | CACNNN/GTG |
| Drd I | GACNNNN/NNGTC |
| EcoH I | /CCSGG |
| EcoN I | CCTNN/NNNAGG |
| EcoO109 I | RG/GNCCY |
| Fal I | (8/13)AAGNNNNNCTT(13/8) |
| Fmu I | GGNC/C |
| Fnu4H I | GC/NGC |
| Hae I | WGG/CCW |
| Hae IV | (7/13)GAYNNNNNRTC(14/9) |
| HgiE II | ACCNNNNNNGGT |
| Hinf I | G/ANTC |
| Hpy8 I | GTN/NAC |
| Hpy99 I | CGWCG/ |
| Hpy188 I | TCN/GA |
| Hpy188 III | TC/NNGA |
| HpyCH4 III | ACN/GT |
| Mae III | /GTNAC |
| Mja IV | GTNNAC |
| Msl I | CAYNN/NNRTG |
| Mwo I | GCNNNNN/NNGC |
| Nci I | CC/SGG |
| Nla IV | GGN/NCC |
| Pas I | CC/CWGGG |
| PflF I | GACN/NNGTC |
| PflM I | CCANNNN/NTGG |
| Pfo I | T/CCNGGA |
| PpuM I | RG/GWCCY |
| PshA I | GACNN/NNGTC |
| Psp03 I | GGWC/C |
| PspG I | /CCWGG |
| Pss I | RGGNC/CY |
| Rsr II | CG/GWCCG |
| SanD I | GG/GWCCC |
| Sau96 I | G/GNCC |
| ScrF I | CC/NGG |
| SexA I | A/CCWGGT |
| Sfi I | GGCCNNNN/NGGCC |
| Sse8647 I | AG/GWCCT |
| Sty I | C/CWWGG |
| StyD4 I | /CCNGG |
| Tat I | W/GTACW |
| Tau I | GCSG/C |
| Tfi I | G/CWGC |
| Tsp45 I | /GTSAC |
| TspR I | CASTGNN/ |
| Tth111 I | GACN/NNGTC |
| UbaM I | TCCNGGA |
| Unb I | /GGNCC |
| VpaK11A I | /GGWCC |
| Xcm I | CCANNNNN/NNNNTGG |
| Xmn I | GAANN/NNTTC |

Useful enzymes with non palindromic recognition
sequences including Typells Restriction
endonucleases

| | |
|---|---|
| AarI | CACCTGCNNNN\hat{ } |
| | GTGGACGNNNNNNNN\hat{ } |
| AceIII | CAGCTCNNNNNNN\hat{ } |
| | GTCGAGNNNNNNNNNNN\hat{ } |
| AciI | C\hat{ }CG C |
| | G GC\hat{ }G |
| AloI | GAACNNNNNNTCCNNNNNNNNNNNN\hat{ } |
| | CTTGNNNNNNAGGNNNNNNN\hat{ } |
| AloI | GGANNNNNNGTTCNNNNNNNNNNNN\hat{ } |
| | CCTNNNNNNCAAGNNNNNNN\hat{ } |
| AspCNI | GCCGC |
| | CGGCG |
| BaeI | ACNNNNGTAYCNNNNNNNNNNNN\hat{ } |
| | TGNNNNCATRGNNNNNNN\hat{ } |
| BaeI | GTTACNNNNGTNNNNNNNNNNNNNNN\hat{ } |
| | \hat{ }NNNNNNNNNNACNNNNGTAYC |
| Bbr7I | GAAGACNNNNNNN\hat{ } |
| | CTTCTGNNNNNNNNNNN\hat{ } |
| BbvI | GCAGCNNNNNNNN\hat{ } |
| | CGTCGNNNNNNNNNNNN\hat{ } |
| BbvII | GAAGACNN\hat{ } |
| | CTTCTGNNNNNN\hat{ } |
| BbvCI | CC\hat{ }TCA GC |
| | GG AGT\hat{ }CG |
| BccI | CCATCNNNN\hat{ } |
| | GGTAGNNNNN\hat{ } |
| Bce83I | CTTGAGNNNNNNNNNNNNNNNN\hat{ } |
| | GAACTCNNNNNNNNNNNNNN\hat{ } |
| BceAI | ACGGCNNNNNNNNNNNN\hat{ } |
| | TGCCGNNNNNNNNNNNNNN\hat{ } |
| BcefI | ACGGCNNNNNNNNNNNN\hat{ } |
| | TGCCGNNNNNNNNNNNNN\hat{ } |
| BcgI | CGANNNNNNTGCNNNNNNNNNNNN\hat{ } |
| | GCTNNNNNNACGNNNNNNNNNN\hat{ } |
| BcgI | GCANNNNNNTCGNNNNNNNNNNNN\hat{ } |
| | CGTNNNNNNAGCNNNNNNNNNN\hat{ } |
| BciVI | GTATCCNNNNNN\hat{ } |
| | CATAGGNNNNN\hat{ } |
| BfiI | ACTGGGNNNNN\hat{ } |
| | TGACCCNNNN\hat{ } |
| BinI | GGATCNNNN\hat{ } |
| | CCTAGNNNNN\hat{ } |
| BmgI | GKGCCC |
| | CMCGGG |
| Bpu10I | CC\hat{ }TNA GC |
| | GG ANT\hat{ }CG |
| BsaXI | ACNNNNNCTCCNNNNNNNNNN\hat{ } |
| | TGNNNNNGAGGNNNNNNN\hat{ } |
| BsaXI | GGAGNNNNNGTNNNNNNNNNNNN\hat{ } |
| | CCTCNNNNNCANNNNNNNNN\hat{ } |
| BsbI | CAACAC |
| | GTTGTG |
| BscAI | GCATCNNNN\hat{ } |
| | CGTAGNNNNNN\hat{ } |
| BscGI | CCCGT |
| | GGGCA |
| BseMII | CTCAGNNNNNNNNNN\hat{ } |
| | GAGTCNNNNNNNN\hat{ } |
| BseRI | GAGGAGNNNNNNNNNN\hat{ } |
| | CTCCTCNNNNNNNN\hat{ } |
| BseYI | C\hat{ }CCAG C |
| | G GGTC\hat{ }G |
| BsgI | GTGCAGNNNNNNNNNNNNNNNN\hat{ } |
| | CACGTCNNNNNNNNNNNNNN\hat{ } |
| BsiI | C\hat{ }ACGA G |
| | G TGCT\hat{ }C |
| BslFI | GGGACNNNNNNNNNN\hat{ } |
| | CCCTGNNNNNNNNNNNNNN\hat{ } |
| BslFI | GTCCCNNNNNNNNNNNNNNN\hat{ } |
| | CAGGGNNNNNNNNNNN\hat{ } |
| BsmI | GAATG CN\hat{ } |
| | CTTAC\hat{ }G |
| BsmAI | GTCTCN\hat{ } |
| | CAGAGNNNNN\hat{ } |
| BsmFI | GGGACNNNNNNNNNN\hat{ } |
| | CCCTGNNNNNNNNNNNNNN\hat{ } |
| Bsp24I | GACNNNNNNTGGNNNNNNNNNNNN\hat{ } |
| | CTGNNNNNNACCNNNNNNN\hat{ } |
| Bsp24I | CCANNNNNNGTCNNNNNNNNNNNNN\hat{ } |
| | GGTNNNNNNCAGNNNNNNNN\hat{ } |
| BspCNI | CTCAGNNNNNNNNN\hat{ } |
| | GAGTCNNNNNNN\hat{ } |
| BspGI | CTGGAC |
| | GACCTG |
| BspMI | ACCTGCNNNN\hat{ } |
| | TGGACGNNNNNNNN\hat{ } |
| BspNCI | CCAGA |
| | GGAGA |
| BsrI | ACTG GN\hat{ } |
| | TGAC\hat{ }C |
| BsrBI | CCG\hat{ }CTC |
| | GGC\hat{ }GAG |
| BsrDI | GCAATGNN\hat{ } |
| | CGTTAC\hat{ } |
| BstF5I | GGATGNN\hat{ } |
| | CCTAC\hat{ } |
| BtgZI | GCGATGNNNNNNNNNN\hat{ } |
| | CTCTACNNNNNNNNNNNNNN\hat{ } |
| BtrI | CAC\hat{ }GTC |
| | GTG\hat{ }CAG |
| BtsI | GCAGTGNN\hat{ } |
| | CGTCAC\hat{ } |
| CdiI | CATC\hat{ }G |
| | GTAG\hat{ }C |
| CjeI | CCANNNNNNGTNNNNNNNNNNNNNNN\hat{ } |
| | GGTNNNNNNCANNNNNNNNN\hat{ } |
| CjeI | ACNNNNNNTGGNNNNNNNNNNNNNN\hat{ } |
| | TGNNNNNNACCNNNNNNNN\hat{ } |
| CjePI | CCANNNNNNNTCNNNNNNNNNNNNNN\hat{ } |
| | GGTNNNNNNNAGNNNNNNNN\hat{ } |
| CjePI | GANNNNNNNTGGNNNNNNNNNNNNN\hat{ } |
| | CTNNNNNNNACCNNNNNNN\hat{ } |
| CspCI | CAANNNNNGTGGNNNNNNNNNNNN\hat{ } |
| | GTTNNNNNCACCNNNNNNNNNN\hat{ } |
| CspCI | CCACNNNNNTTGGNNNNNNNNNNNNN\hat{ } |
| | GGTGNNNNNAACNNNNNNNNNNN\hat{ } |
| CstMI | AAGGAGNNNNNNNNNNNNNNNNNNNN\hat{ } |
| | TTCCTCNNNNNNNNNNNNNNNNNN\hat{ } |
| DrdII | GAACCA |
| | CTTGGT |
| EciI | GGCGGANNNNNNNNNNN\hat{ } |
| | CCGCCTNNNNNNNNN\hat{ } |
| Eco31I | GGTCTCN\hat{ } |
| | CCAGAGNNNNN\hat{ } |
| Eco57I | CTGAAGNNNNNNNNNNNNNNNN\hat{ } |
| | GACTTCNNNNNNNNNNNNNN\hat{ } |
| Eco57MI | CTGRAGNNNNNNNNNNNNNNNN\hat{ } |
| | GACYTCNNNNNNNNNNNNNN\hat{ } |
| Esp3I | CGTCTCN\hat{ } |
| | GCAGAGNNNNN\hat{ } |
| FauI | CCCGCNNNN\hat{ } |
| | GGGCGNNNNNN\hat{ } |
| FinI | GGGAC |
| | CCCTG |
| FokI | GGATGNNNNNNNNN\hat{ } |
| | CCTACNNNNNNNNNNNNN\hat{ } |
| GdiII | C\hat{ }GGCC R |
| | G CCGG\hat{ }Y |
| GsuI | CTGGAGNNNNNNNNNNNNNNNN\hat{ } |
| | GACCTCNNNNNNNNNNNNNN\hat{ } |
| HaeIV | GAYNNNNNRTCNNNNNNNNNNNNNN\hat{ } |
| | CTRNNNNNYAGNNNNNNNNN\hat{ } |
| HgaI | GACGCNNNNN\hat{ } |
| | CTGCGNNNNNNNNNN\hat{ } |
| Hin4I | GAYNNNNNVTCNNNNNNNNNNNNN\hat{ } |
| | CTRNNNNNBAGNNNNNNNN\hat{ } |
| Hin4I | GABNNNNNRTCNNNNNNNNNNNNN\hat{ } |
| | CTVNNNNNYAGNNNNNNNN\hat{ } |
| Hin4II | CCTTC |
| | GGAAG |
| HphI | GGTGANNNNNNNN\hat{ } |
| | CCACTNNNNNNN\hat{ } |
| HpyAV | CCTTCNNNNNN\hat{ } |
| | GGAAGNNNNN\hat{ } |
| Ksp632I | CTCTTCN\hat{ } |
| | GAGAAGNNNN\hat{ } |
| MboII | GAAGANNNNNNNN\hat{ } |
| | CTTCTNNNNNNN\hat{ } |
| MlyI | GAGTCNNNNN\hat{ } |
| | CTCAGNNNNN\hat{ } |
| MmeI | TCCRACNNNNNNNNNNNNNNNNNNNN\hat{ } |
| | AGGYTGNNNNNNNNNNNNNNNNNN\hat{ } |
| MnlI | CCTCNNNNNNN\hat{ } |
| | GGAGNNNNNN\hat{ } |
| Pfl1108I | TCGTAG |
| | AGCATC |
| PleI | GAGTCNNNN\hat{ } |
| | CTCAGNNNNN\hat{ } |
| PpiI | GAACNNNNNCTCNNNNNNNNNNNNN\hat{ } |
| | CTTGNNNNNGAGNNNNNNNN\hat{ } |
| PpiI | GAGNNNNNGTTCNNNNNNNNNNNN\hat{ } |
| | CTCNNNNNCAAGNNNNNNN\hat{ } |
| PsrI | GAACNNNNNNTACNNNNNNNNNNNN\hat{ } |
| | CTTGNNNNNNATGNNNNNNN\hat{ } |
| PsrI | GTANNNNNNGTTCNNNNNNNNNNNN\hat{ } |
| | CATNNNNNNCAAGNNNNNNN\hat{ } |
| RleAI | CCCACANNNNNNNNNNNN\hat{ } |
| | GGGTGTNNNNNNNNN\hat{ } |
| SapI | GCTCTTCN\hat{ } |
| | CGAGAAGNNNN\hat{ } |
| SfaNI | GCATCNNNNN\hat{ } |
| | CGTAGNNNNNNNNN\hat{ } |
| SimI | GG\hat{ }GTC |
| | CC CAG\hat{ } |
| SspD5I | GGTGANNNNNNNN\hat{ } |
| | CCACTNNNNNNNN\hat{ } |
| Sth132I | CCCGNNNN\hat{ } |
| | GGGCNNNNNNNN\hat{ } |
| StsI | GGATGNNNNNNNNNN\hat{ } |
| | CCTACNNNNNNNNNNNNNN\hat{ } |
| TaqII | GACCGANNNNNNNNNNN\hat{ } |
| | CTGGCTNNNNNNNNN\hat{ } |
| TaqII | CACCCANNNNNNNNNNN\hat{ } |
| | GTGGGTNNNNNNNNN\hat{ } |
| TsoI | TARCCA |
| | ATYGGT |
| TspDTI | ATGAANNNNNNNNNNN\hat{ } |
| | TACTTNNNNNNNNN\hat{ } |
| TspGWI | ACGGANNNNNNNNNNN\hat{ } |
| | TGCCTNNNNNNNNN\hat{ } |
| Tth111II | CAARCANNNNNNNNNNN\hat{ } |
| | GTTYGTNNNNNNNNN\hat{ } |
| UbaF2I | GAAAYNNNNNRTG |
| | CTTTRNNNNNYAC |
| UbaF3I | CACNNNNNNTCC |
| | GTGNNNNNNAGG |
| UbaF4I | GAANNNNNNNTTGG |
| | CTTNNNNNNNAACC |
| UbaF5I | CTGATG |
| | GACTAC |
| UbaF6I | GCGAC |
| | CGCTG |
| UbaPI | CGAACG |
| | GCTTGC |

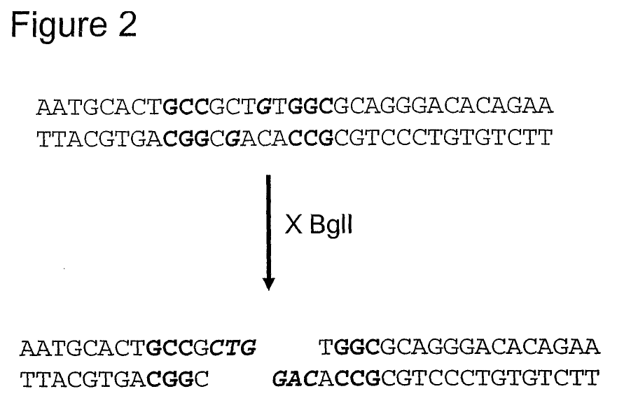

By way of further example, interrupted palindrome restriction enzymes are characterised in that they have a recognition sequence that flanks a sequence of characteristic length. Any combination of bases can therefore be found in the cohesive end (the resulting single stranded overhanging end) but the combination will, again, always be characteristic for a given DNA fragment being cleaved. FIG. 2 shows how an interrupted palindrome restriction endonuclease operates to cleave a DNA fragment, by reference to the enzyme Bgl I. Bgl I as shown here has a recognition site (shown in bold) that flanks 5 bases, but only three of these form the single stranded overhanging end, which can therefore have a sequence which is any one of 64(4³) possibilities (again, dependent on the sequence of the molecule being cleaved). In FIG. 2 the cut points in the upper and lower strands are shown in bold and italic. Again, interrupted palindrome restriction enzymes other than Bgl I are available and will be known to the skilled person, and examples are provided in Table 1. The use of enzymes such as these with a displaced cleavage site allows for the point of linearization of the circular molecule to be directed by the indexing molecule, but to be located within part of the circularised molecule being derived from the ds DNA molecule being labelled. In this way, single stranded overhanging ends are provided on each end of the indexed ds DNA molecule, and those single stranded overhanging ends have sequences derived from the original molecule to be labelled. In addition, linearization in this way results in the indexing molecules already incorporated into the ds DNA molecule being maintained together and proximal to one end of the molecule where they go to make up a bar-code style label.

In order to ensure that, on cleavage with the displaced cleavage restriction enzyme, the indexed circularised molecule is only cleaved once and is therefore linearised with an intact bar-code label proximal to one end thereof, it is necessary that only one of the indexing molecules ligated in each round comprises a recognition site for the displaced cleavage restriction enzyme. In some embodiments, and for the first round of indexing, this may be achieved by ensuring that the single stranded overhanging ends of the ds DNA molecule are both provided on the same strand such that at one end of the DNA molecule there is a 5′ overhang and at the other end of the DNA molecule there is a 3′ overhang. Such a double stranded DNA molecule can be ligated to a pool of indexing molecules in which the pool comprises a first category of indexing molecules with 5′ overhanging single stranded ends, and a second category of indexing molecules with 3′ overhanging single stranded ends. Only one of the two categories of indexing molecules further comprises the recognition site for the displaced cleavage restriction endonuclease.

In other embodiments, and for subsequent rounds of indexing, the pool of indexing molecules again comprises a first, and a second, category of indexing molecules. The first category of indexing molecules comprises the recognition site for the displaced cleavage restriction enzyme, and the second category does not comprise the recognition site. In this embodiment, both categories of indexing molecules may have single stranded overhanging ends on the appropriate strand (i.e., 5′ or 3′ overhangs) in order to be complementary to the single stranded overhangs of the linearised, labelled ds DNA molecule. When a 50:50 mixture of the first and second category of indexing molecules are used, on average, 25% of the ds DNA molecules to be labelled will, following ligation to the pool of indexing molecules, include one indexing molecule with the appropriate restriction enzyme recognition site, and one indexing molecule without the recognition site. DNA molecules which incorporate two indexing molecules from the first category (i.e., comprising the recognition site) will be digested upon linearization, and will lose their label and will be, therefore, invisible during detection stages of the labelling method. Individual ds DNA molecules which incorporate two indexing molecules of the second category (i.e., having no recognition site for the restriction enzyme) will remain circular and can be distinguished from linear labelled molecules on this basis.

In an alternative approach, the restriction enzyme recognition site is only created upon circularization of an appropriately ligated molecule. Thus, neither indexing molecule has an intact recognition site, the recognition site being created by bringing the correct two indexing molecules together on circularization. Alternatively, the indexing molecules can be designed such that two identical indexing molecules coming together form a restriction site that can be used to select for correct combinations by cleavage of incorrect molecules.

Whilst other means to avoid incorrect pairing will be apparent to the skilled person, any aberrant labelling of ds DNA molecules caused by inappropriate pairing of indexing molecules that does occur will be in the minority and can be excluded from analysis of labelled molecules by analysis of a sufficiently large number of individual molecules.

As noted, the labelling procedure requires circularisation of ds DNA molecules ligated to indexing molecules in order to bring indexing molecules at each end of the ds DNA molecule together. In some embodiments, the outer ends of the indexing molecules ligated to the ds DNA molecule are rendered compatible for joining to each other in a ligation reaction. Rendering of the ends of the indexing molecules can be achieved by cleavage with a suitable endonuclease to provide compatible cohesive ends. Accordingly, in some embodiments, the indexing molecules of the pool of indexing molecules comprise recognition sites for a restriction enzyme, which enzyme is capable of cutting ds DNA to provide single stranded overhanging ends. Suitable restriction enzymes will be apparent to the skilled person, as will position of the restriction enzyme recognition sites in the indexing molecules. For indexing molecules which include the above-noted recognition site for the displaced cleavage restriction enzyme, the recognition site of that displaced cleavage restriction enzyme should be located between the further restriction enzyme site used for rendering the ends suitable for circularisation, and the complementary single stranded end of the indexing molecule. In this way, the recognition site for the displaced cleavage restriction enzyme is not removed or destroyed upon rendering the ends suitable for circularisation.

In other embodiments, rendering of the ends for circularisation comprises phosphorylating, in vitro, one strand of the attached indexing molecule, in order to provide a substrate for blunt ended or cohesive ended ligation. In some preferred embodiments of the methods of the invention, adaptor-like molecules, including indexing molecules, are purified from a biological source, and will therefore be phosphorylated. In some embodiments, such adaptor-like molecules do not need to be phosphorylated prior to use in ligation reactions and can provide for enhanced efficiency of ligation. In other embodiments it is preferred to maintain the ends of the ligation product in an unphosphorylated state until immediately before circularisation, even where indexing molecules are purified rather than synthesised. This can be achieved by removing phosphate at an earlier step, or by modification of ends using synthetic oligonucleotides which remain unphosphorylated until rendered for circularisation. In more preferred embodiments, adaptor-like molecules (including indexing molecules) may be provided as single stranded molecules derived, for example, from bacteriophage vectors. Single stranded DNA for use as adaptor-like molecules is readily isolatable according to methodology well known to the skilled person, and can provide for enhance fidelity in the ligase reaction. The provision of adaptor-like molecules in single stranded form also avoids potential problems with co-purifying bacterial DNA, which can otherwise contaminate labelling and sequencing reactions.

For example, ØX174 DNA may be modified and labelled in order to provide for indexing molecules. The single stranded DNA may be rendered partially double stranded for use as an indexing molecule.

Where single stranded indexing molecules are incorporated into the ds DNA molecule to be labelled in the ligation step, it is necessary to ‘fill in’ the second strand in order to render the ends of the ligation production suitable for circularisation. This is achievable using, for example, T4 DNA polymerase or Taq polymerase under conditions that will be readily apparent to the skilled person. It is important that the ‘filling in’ step to be carried out at temperatures at or below approximately 12° C. in order to inhibit the enzyme's nuclease activity, or to use conditions under which there is net filling in rather than digestion. Appropriate conditions will be readily apparent to the skilled person. Filling in may occur before of after ligation of the indexing molecules to the ds DNA. In some embodiments, partial filling occurs before ligation, and the process is completed after indexing, thereby making restriction sites available only after the final filling in stage.

In yet further embodiments, no step of rendering is required, for example, if the indexing molecules are appropriately phosphorylated before being incorporated into the ds DNA molecule to be labelled. In one embodiment, indexing molecules may have incompatible overhanging ends, and the step of circularization requires the addition of a linker adaptor that joins the two ends together thereby circularizing the adapted ds DNA molecule. This embodiment is advantageous in that the linker adaptor may provide the recognition site for the enzyme used subsequently to linearise the circular molecule.

As noted above, the indexing molecules are labelled differentially according to the sequence of their single stranded ends complementary to the single stranded overhanging ends of the ds DNA molecule to be labelled. Any suitable detectable label can be used for the indexing molecules. For example, in some embodiments, the indexing molecules are labelled differentially with fluorescent molecules such as FAM (carboxyfluoroscene), JOE (carboxy-4′,5′-dichloro-2′,7′-dimethoxyfluoroscene), TAMRA (carboxytetramethylrhodamine), ROX (carboxy-X-rhodamine), Cye dyes, Alexafluor dyes and others (see, for example, Handbook of Fluorescent Probes and Research Chemicals by Richard P Haugland, 6^{th} Edition, Molecular Probes Inc Catalogue, Chapter 8.2). In addition, energy transfer dyes and systems may be used such as fluorescence energy resonance transfer systems (FRET).

In alternative embodiments, indexing molecules can be labelled with fluorescent nanoparticles such as quantum dots available from Quantum Dot Corporation. Other embodiments include labelling the indexing molecules with, for example, short lived isotopes e.g., technicium, or with topological markers such as branched polymer structures. Reagents suitable for microscopy labelling and detection will, in general, be suitable if conjugated to indexing molecules, for example, immunogold. Topological markers in the form of branched structures can be in the form of oligonucleotides that are part paired with the indexing molecule thereby leaving an unannealed tail for further labelling or extension. Of course, and as will be apparent to the skilled person, any combination of the above-noted labels may be used for different indexing molecules with the pool of indexing molecules. Indexing molecules may be labelled directly, after their manufacture or, in a preferred embodiment, may be labelled by incorporation of labelled nucleotides, according to methods well known to skilled persons e.g., using kits available from, for example, Molecular Probes Inc.

It is preferred that the indexers are labelled so that they can be discriminated. Many different labelling strategies are known in the art and include fluorescent dyes, quantum dots (nanoparticles) and shape or size labels. Discrimination as single molecules is especially desirable so that their end sequences can be determined. Strategies for discrimination can be either spatial or spectral or a mixture. Spatial involves either different shaped molecules separated by position to produce a code or different colours (fluorescent dyes or particles) separated by position. Spectral involves having multiple possible colours at a single position and the exact combination encoding particular information. In the case of the latter, for example, 2 dyes allow 3 possibilities even without blending i.e. 100% first dye or 100% second dye or 50% each dye. More blending e.g. 25% of either dye with 75% of the other dye or more dyes increase the possibilities exponentially per addition and therefore the number of possible codes. Labelling can either be direct or indirect or a mixture of both. Direct labelling has the advantage that further procedures to allow detection, are not required after the final indexing steps. Adequate and/or rapid discrimination between single molecules require in general larger indexing molecules. These are necessary either to incorporate sufficient amounts of different labels for adequate detection if spectral coding is used or to allow sufficient resolution if spatial coding is used. As discussed, the mass associated with larger indexers is unfavourable for reactions having more complex mixtures of indexers. It is sometimes beneficial therefore to have secondary detection of all or some of the indexers. Smaller indexers can then be used and it is required that there is a means for the detection whilst retaining spatial or spectral discrimination. The simplest case for secondary detection involves a branching oligonucleotide. An oligonucleotide with 2 5′ ends and 1 3′ end for example can be used as an indexer as above. Standard hybridisation techniques can then be used to detect a sequence in the 5′ branch that does not participate in the indexing reactions. Matched pairs of probes and 5′ branches are used so that each 5′ branch can be separately detected and in turn identify the original indexer that participated in the reactions that gave rise to a particular molecule. The kinetics of hybridisation favour the use of high concentrations of probe and high molecular weight probes compared to the current kinetics of the indexing reactions.

Indexing molecules for bar-coding may be of any length compatible with their functional requirements noted above. In a preferred embodiment, indexing molecules are at least 4 kb long, but in other embodiments shorter indexing molecules can be used. The length of the indexing molecule can influence how the bar-code label is subsequently detected, and can have an important effect on the number of DNA molecules that can be detected and scored during the detection procedure described in further detail below. In general, a longer indexing molecule allows for easier resolution of individual labels within a bar-code and can allow for multiple labels per indexing molecule, for example one indexing molecule may have 2 or more dyes in a particular collinear order. Shorter indexing molecules may be detected and resolved using higher power magnification, but this necessitates a smaller field of view, resulting, in turn, in scoring fewer label molecules of each sequence per field of view.

Indexing molecules may be single stranded or double stranded or part single stranded/part double stranded and may be chemically synthesised in vitro or isolated from a biological system. Indexing molecules may be provided in the form of specifically engineered plasmids or other vectors, for example, bacteriophage vectors. In a preferred embodiment, engineered bacteriophage vector φx174 can be used.

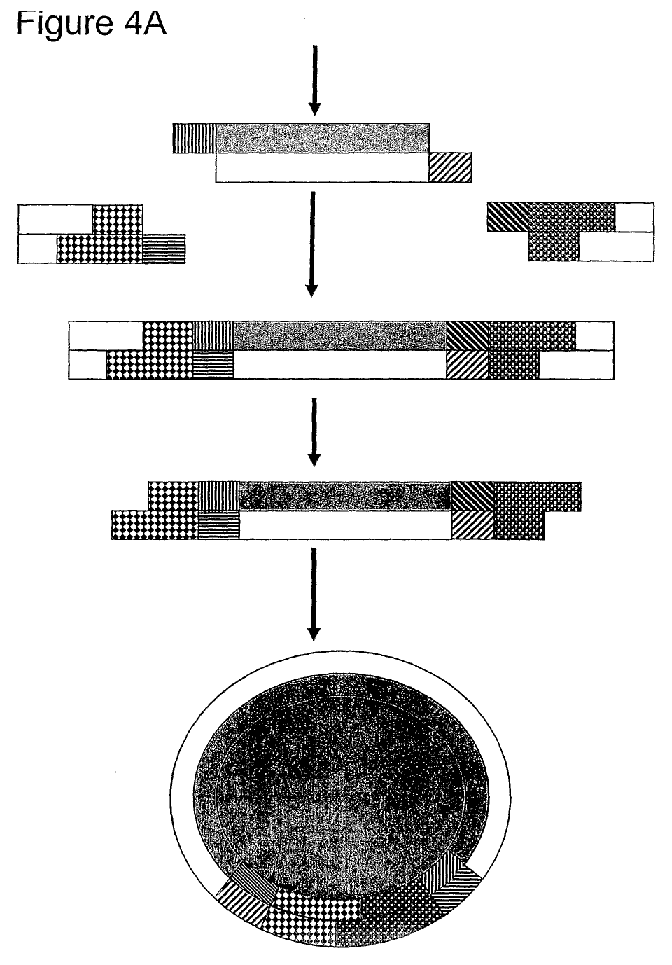

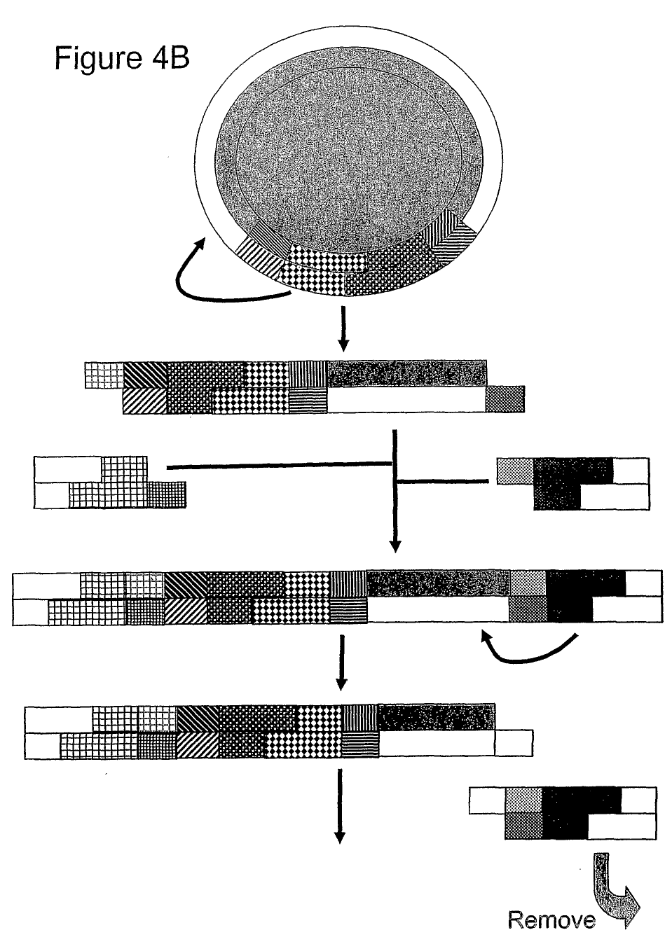

The process of labelling one end of a ds DNA molecule with indexing molecules will now be described in detail with reference to FIGS. 4A and 4B. However, modifications in precise design of indexing molecules and positioning of restriction enzyme recognition and cleavage sites will occur to the skilled person. For example, it is possible to arrange for the cleavage site of the displaced cleavage restriction enzyme to be precisely at the junction between the indexing molecule and the remainder of the molecule being labelled. In this way, known bases may be added to the ds DNA end from the indexing molecule.

FIG. 4A shows a ds DNA molecule with single stranded overhanging ends. The single stranded overhanging ends provided on the ds DNA molecule illustrated are 5′ overhanging ends, but could equally well be 3′ overhanging ends. Alternatively, the overhanging end at one end of the molecule could be a 3′ overhanging end and the overhanging end at the other end of the molecule could be a 5′ overhanging end. The overhanging ends may be of any suitable length as would be understood by the skilled person, for example, but not limited to, 1, 2, 3, 4 or 5 nucleotide bases long. Alternatively, the single stranded overhanging ends may be lengthened according to the methods of the invention to provide overhanging ends of, for example, 6, 7, 8, 9, 10, 11, 12, 13, 14, 15, 16, 17 or 18 bases long. In a preferred embodiment, the overhanging ends are 8 bases long.

The ds DNA molecule having single stranded overhanging ends is incubated with a pool of indexing molecules having single stranded ends complementary to the single stranded overhanging ends of the ds DNA molecule. The pool of indexing molecules may comprise different indexing molecules having all possible combinations of sequences at their single stranded ends, or a mixture of indexing molecules having a subset of the possible sequences in their single stranded ends. Each indexing molecule within the pool is labelled, and the labels are distinguishable for indexing molecules with different sequences in the single stranded end.

The mixture is incubated under conditions suitable for ligation between the indexing molecules and the ds DNA molecule to be labelled. Routine conditions for ligation may be applied according to the DNA ligase enzymes manufacturer's instructions, or according to conditions that can be readily determined by the skilled person. In some embodiments, T4 DNA ligase is used. In other embodiments, other ligase enzymes may be used, for example, the thermostable DNA ligase pfu available from Stratagene, or Taq DNA ligase available from NEB. Other ligases that can be used include E. coli DNA ligase also available from NEB.

The resulting linear ligation product comprises the ds DNA molecule to be labelled ligated to indexing molecules at each end. If necessary, and as described above, the outer ends of the linear ligation product are then rendered suitable for annealing and ligation to each other, for example, by cleavage with one or more restriction endonuclease whose recognition sites are found within the indexing molecule sequences to generate compatible cohesive ends. The so rendered molecule is circularised by incubation under conditions suitable for ligation to occur, thereby joining the outer ends of the linear ligation product from the previous step together and, importantly, bringing the two indexing molecules incorporated into the ds DNA molecule to be labelled, together.

Following circularisation, the circular ligation product is then incubated with the displaced cleavage restriction enzyme whose recognition site is located within one of the indexing molecules incorporated into the ds DNA molecule (see FIG. 4B). Because of the displaced positioning of the cleavage site of the enzyme, the circular molecule is cleaved at a position derived originally from the ds DNA molecule to be labelled to provide a linear molecule with single stranded overhanging ends having a sequence characteristic of the ds DNA molecule sequence.

The linearised product comprises, in order from one end, a short fragment of the ds DNA molecule to be labelled comprising a single stranded overhanging end, fused to one of the indexing molecule incorporated at one end of the original linear ds DNA molecule, fused to the second indexing molecule ligated at the other end of the original linear ds DNA molecule, fused directly to the remainder of the ds DNA molecule which, itself, has a single stranded overhanging end at its other end.

The linear indexing molecule/ds DNA molecule ligation product resulting from these steps provides a substrate for further rounds of incorporation of indexing molecules by repeating the ligation, circularisation and linearization steps. Subsequent rounds of incorporation of indexing molecules in this way provides for a string of indexing molecules located proximal to one end of the ds DNA molecule to be labelled. The identity of the indexing molecules incorporated into this multifaceted label is directed by the sequence of the overhanging ends following linearization at each round and, in this way, a bar-code label characteristic of the ds DNA molecule is added proximal to one end of that molecule. In the final round of labelling it is not always necessary to carry out the circularization/cleavage steps and the final labelled product has a single indexing molecule at one end and one or more indexing molecules at the other end.

The ds DNA molecules labelled according to the methods described are suitable templates for deriving sequence information from the end distal to the bar-code label using adaptor-mediated sequencing techniques. The final stage of labelling (regardless of the number of rounds of incorporation of indexing molecules) can be arranged to reveal a single stranded overhanging end distal to the bar-code label, the sequence of which is derived from the ds DNA molecule, and is therefore characteristic thereof. Nucleic acid bases within the single stranded overhanging end can be determined directly by ligation to a pool of sequencing adaptors, wherein members of the pool of sequencing adaptors have at least a subset of all possible nucleic acid sequences in a complimentary single stranded end suitable for annealing to the single stranded overhanging end of the labelled ds DNA molecule. Each member of the pool of sequencing adaptors is labelled, and each member having the same sequence in its complementary single stranded end has the same label. Members of the pool having different sequences in their complementary single stranded ends have distinguishable labels, and can therefore be distinguished from one another.

Labelled ds DNA molecules are therefore ligated to the pool of sequencing adaptors, and can subsequently be detected according to methods described in greater detail below. Detection and analysis of labelled ds DNA molecule/sequencing adaptor ligation products involves identification of one or more labelled ds DNA molecules having the same bar-code label. As noted previously the bar-code label is characteristic of the sequence of the fragment being labelled, and if sufficient rounds of indexing are employed during the indexing method, then any one bar-code label will uniquely identify fragments of ds DNA having the same sequence. It will be apparent to the skilled person, depending upon the length of single stranded overhanging ends used during the labelling method for recognition by indexing molecules, and on the complexity of the starting material, and other factors, how many rounds of indexing are required in order to provide for unique identification of a DNA sequence according to its bar-code label.

The skilled person will readily be able to determine the number of indexing rounds, the number of starting points for indexing an average fragment size and an amount of size selection or size reduction appropriate to their needs. When sequence is known and the objective is to resequence this can be determined by prior electronic analysis of the target sequence. For example, there are approximately 400000 BamHI sites and 800000 BgLII sites as detected in the human genome by “fuzznuc” a program of the EMBOSS suite (Rice, P, Longden, I, and Bleasby, A “EMBOSS”: the European Molecular Biology Open Software Suite”, Trends in Genetics June 2000, vol 16, No 6, pp 276-277, incorporated herein by reference for all purposes). The location of these sites and their surrounding sequence is known. A minimum number of nucleotides that need to be identified per fragment end is 9 to 10 respectively because this exceeds the maximum number of permutations that could be found on all possible ends. These could be labelled with less than three indexing molecules. Sites that are more or less frequent would require correspondingly more or less indexers for labelling. In practice, the human genome contains repetitive sequences which reduce the number of different sequences that are to be found. More information is needed to distinguish members of the repeat class however as these are likely to be the same or very similar for long stretches of sequence. Although in such circumstances it is possible to increase the number of indexing rounds it is preferable to start indexing at more different places to increase the number of start points in unique sequence that can be easily distinguished and then read from these places through repeat sequences as necessary. The lengths of the reads and therefore the minimum lengths of the fragments to be determined has to be sufficient to span the repeats, typically in the order of a few hundred bases to several tens of kilobases. Longer range repeats are distinguished by naturally occurring nucleotide differences that occur approximately on average every 500 base pairs.

Shorter sequences than the human genome require correspondingly less indexing. The same considerations that apply to known sequence also apply to unknown sequence so that sequencing can be set up by analogy with the human. If longer average repeats are found then longer fragments would be required. If unknown sequence proves to be complex with fewer repeats then shorter fragments can be exploited.

In most embodiments no more than 4 rounds of indexing are necessary and preferably multiple types of start points such as those produced by different restrictions endonucleases are exploited.

Identified ds DNA molecule fragments having a common bar-code label (and therefore a common sequence) can be analysed to determine which of the pool of sequencing adaptors is ligated to the end distal to the bar-code label. Identification of the specific sequence that has annealed and ligated to the labelled ds DNA molecule can be used to determine the identity of one or more nucleic acids bases in the single stranded overhanging end of the ds DNA molecule by reference to the sequence of the single stranded end of the sequencing adaptor. In some circumstances, all of the nucleic acid bases in the single stranded overhanging end of the labelled ds DNA molecule will be determined in this way. In other circumstances, a subset of the bases in the single stranded overhanging ends of the labelled ds DNA molecule will be determined in this way. In certain embodiments, some information concerning the sequence in the single stranded overhanging ends of the labelled ds DNA molecule may already be known, and in such circumstances, the pool of sequencing adaptors may not need to include all possible combinations of single strand end sequence for annealing and ligation to the single stranded overhanging end of the labelled ds DNA molecule.

Adaptor-mediated sequencing of labelled ds DNA molecules having single stranded overhanging ends employs many of the techniques described in PCT patent application WO 95/20053 in the name of Medical Research Council, whose contents are incorporated herein by reference for all purposes.

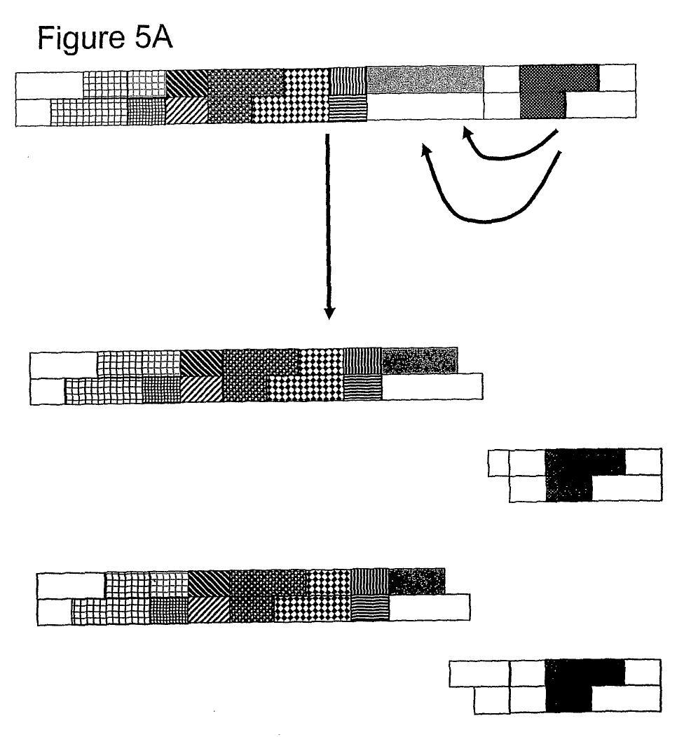

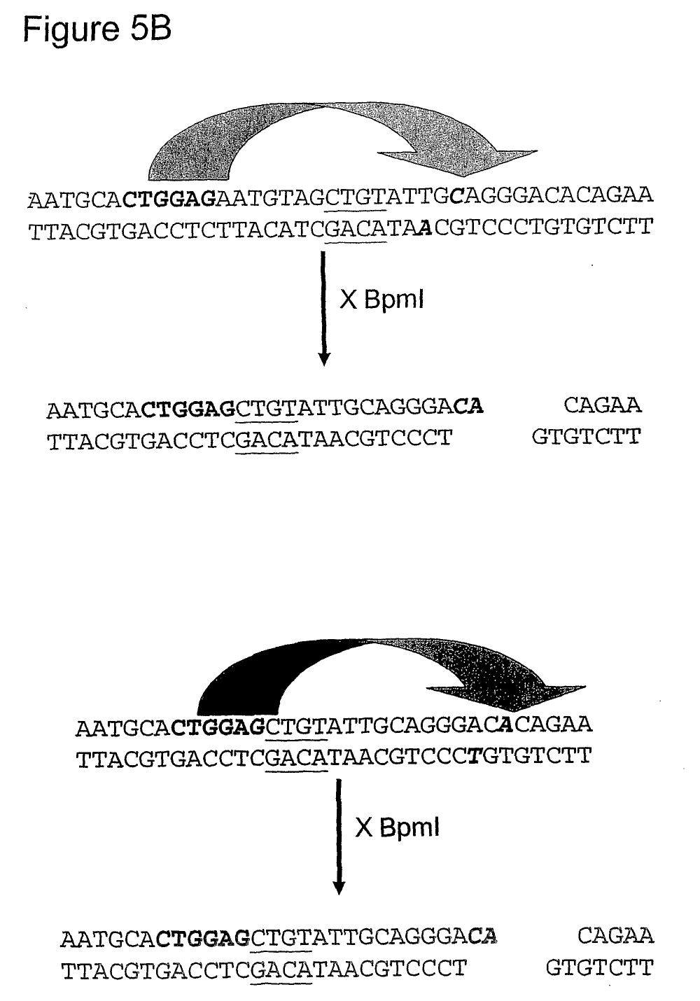

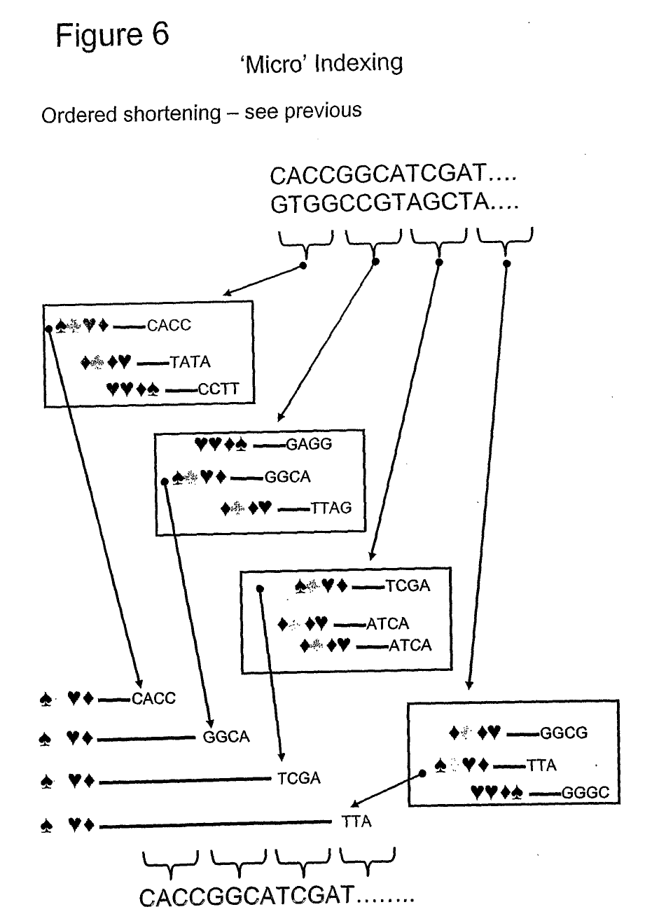

In order to obtain further sequence information from positions internal to the labelled ds DNA molecule, such labelled molecules can be subjected to one or more rounds of controlled size reduction according to the methods described herein. The method of controlled size reduction is designed to shorten the labelled ds DNA molecules by a controlled number of bases in each round, from one end only i.e., the end distal to the bar-code label. The process of controlled size reduction of labelled ds DNA molecules is depicted in FIGS. 5A and 5B. FIG. 5A shows a bar-code labelled ds DNA molecule (with the bar-code depicted at the left hand end). For more efficient controlled size reduction and subsequent steps, it is preferable to ensure that the labelled end of the ds DNA molecule is blocked from further ligation or cleavage whilst ensuring that the opposite end of the molecule is free to proceed. Any suitable capping strategy, as will be well appreciated by the skilled person, may be used for this step. For example, the bar-code labelled ds DNA molecule may be capped on the end proximal to the bar-code by chemical modification or ligation to an adaptor like molecule in order to prevent further participation in subsequent steps. Although it is preferable to ensure maximum capping of the labelled end, rare failures (due e.g., to less than 100% efficiency of ligation) can be tolerated because such failures will subsequently be detected as minority events.

In some embodiments certain steps can be taken to ensure that only the labelled end is capped, so that the opposite end remains free to undergo controlled size reduction and/or sequencing. For example, appropriate design of the indexing molecules used in the final round of labelling can be used to provide, directly or indirectly for a specific end sequence on the labelled end that differs from the end sequence of the opposite end. The specific end sequence provides for a target for capping with an adaptor like molecule which can anneal only to that sequence.

In order to ensure that the labelled end is not shortened during steps of controlled size reduction, some embodiments require that a different displaced restriction site enzyme is used for the labelling steps from that used for controlled size reduction (as discussed below). In alternative embodiments the labelled end is allowed to be shortened since indexing molecules can be designed so that shortening does not disrupt the detectable label. In such circumstances, for example where the indexing molecules are significantly longer than the region to be removed by controlled size reduction, shortening at both ends of the molecule need not impact on the ability to read the bar code.

The uncapped end (i.e. the end distal to the bar-code label) is ligated to a pool of adaptor molecules. The adaptor molecules of the pool are characterised in that they comprise at least a subset of all possible complementary single stranded ends for annealing and ligation to the single stranded overhanging ends of the ds DNA molecules in the sample, and further comprise a recognition site for restriction enzymes having a cleavage site that is physically displaced from its recognition site (for example the type IIs restriction enzymes or interrupted palindrome restriction enzymes described in detail above). Ligation of the labelled ds DNA molecule to one of the adaptor molecules in the pool results in a ligation product having the recognition site for the displaced cleavage restriction enzyme located in a portion derived from the adaptor molecule, and the cleavage site for that enzyme at a position in the ligation product derived from the labelled ds DNA molecule. Subsequent cleavage of the ligation product with the displaced cleavage restriction enzyme (shown in FIG. 5A) results in “cutting back” into the ds DNA molecule in order to reveal a new single stranded overhanging end derived from the labelled ds DNA molecule itself. By appropriate design of the adaptor molecules of the pool, the position of the cleavage site within the ds DNA molecule can be designed, and the number of bases removed from the ds DNA molecule can be preselected. For example, using an enzyme that cuts at a position that is 16 bases away from its recognition site (for example, Bpm I) can result in a controlled size reduction of between one base and 15 bases depending on the position of the recognition site within the adaptors in the pool. The actual number of bases removed depends on how the site for the endonuclease is brought into juxtaposition with the end sequence. For example, the end sequence could be blunt ended or it could have a 5′ or a 3′ overhang. In some embodiments the sequence at the end of the ds DNA molecule could be used in part to form the site for the endonuclease. Each situation has a bearing on the actual number of bases removed as will be appreciated by the skilled person. In the simplest cases where the adaptor molecule makes up the missing strand in either a 3′ or 5′ overhang or the end is blunt ended and in all cases the site starts immediately on the end of the fragment to be cut only, 14 bases are removed since the final 2 bases remain as a 2 base overhang on the cut fragment.

Having achieved one round of controlled size reduction in the way described, it would be apparent to the skilled person that what remains is a labelled ds DNA molecule having a single stranded overhanging end whose sequence is characteristic of the equivalent part of the ds DNA molecule originally labelled, and which can be sequenced according to the method described above using adaptor-mediated sequencing techniques. In this way, sequence information from controlled positions within the labelled ds DNA molecule can be obtained. Labelled ds DNA molecules can undergo one or more rounds of controlled size reduction in order to expose more nucleic acid bases for sequencing by adaptor-mediated sequencing techniques, and according to the design of the adaptor molecules in the pool, larger or smaller controlled size reduction steps can be taken in each round. Moreover, by controlling the size of single stranded overhanging ends of the adaptors in the pool and the position of the recognition site for the displaced cleavage restriction enzyme, the skilled person is able to design means for determining one or more bases at any one time by adaptor-mediated sequencing. Accordingly, by appropriate design, the skilled person is able to determine conditions that will allow for one or more bases to be sequenced at any one time depending on the size of the single stranded overhanging ends of the labelled ds DNA molecule generated in the controlled size reduction steps. Such overhanging ends may be 1, 2, 3, 4, 5, 6, 7, 8 or more bases long depending on the design of the experiment (and depending or not whether single stranded overhanging end lengthening according to the methods described herein is employed).

Longer single stranded overhanging ends generated by the controlled size reduction steps allow for greater sequence information to be provided at each stage of adaptor-mediated sequencing steps, but exponentially increase the complexity of the sequencing step since, for each added base, the number of sequencing adaptors that are included in the pool of sequencing adaptors is multiplied by a factor of 4.

In order to provide sequencing of multiple different ds DNA molecules in a mixed sample (parallel sequencing), the methods of the invention may be adapted so that each different ds DNA molecule in the sample is end labelled with a bar-code and then subjected to varying degrees of controlled size reduction. Thus, following bar-code labelling of two or more different ds DNA molecules in a mixture, that mixture may be subjected to multiple rounds of controlled size reduction according to the methods described herein. In order to obtain sequence information for each of the labelled ds DNA molecules from a plurality of positions in each molecule, the mixture may be sampled following two or more rounds of controlled size reduction. Each sample taken from the mixture may be spread on, e.g., a microscope slide for visual or digital analysis. The visual or digital analysis is used in order to identify and/or score molecules having the same bar-code in order that the sequencing adapter ligated to molecules having the same bar-code and shortened to the same extent can be identified. In this way, for each different and individual bar-coded molecule in the sample, a ligated sequencing adapter, and therefore sequence can be determined. Although due to less than 100% efficiency in some of the steps of the method, it is not expected that all molecules having the same bar code and having undergone the same degree of controlled size reduction will have the same sequencing adapter ligated thereto, errors are in the minority in such a sample and can be excluded from consideration based upon observed frequency of sequence adapter ligation.

In an alternative embodiment, the bar-code labelled mixture of ds DNA molecules is separated into pools before controlled size reduction. Each pool is then subjected to controlled size reduction to a different extent, either by conducting different numbers of rounds of controlled size reduction, or by differential design of adapters mediating the size reduction for each pool. As a result, each pool will contain a mixture of bar-code labelled ds DNA molecules that have been shortened to the same extent. The samples are then spread on e.g., a microscope slide and analysed in the same way as noted above. In alternative embodiments, molecules may be analysed by flowing in a pulsed manner, e.g. by electrophoresis, past a detection system.

In either method, visual or digital inspection of spread pools or samples allows for identification of multiple sets of different bar-coded ds DNA molecules, each having a different sequence. Each bar-coded sequence is represented in each sample or pool multiple times and may be scored for the sequencing adapter ligated at the opposite end to the bar-code. Each sample or pool therefore provides sequence information for multiple different DNA molecules at a single position, and different samples or pools provide sequence information for the same bar-coded ds DNA molecules at a different position. Thus, by combining the methods of parallel ds DNA labelling, controlled size reduction and adaptor-mediated sequencing described herein, the invention provides a means for massively parallel sequencing of multiple molecules in a mixture.

It will be appreciated that controlled size reduction to defined positions is unnecessary where all possible end positions to provide sequence are already in existence or can easily be created. In one particularly preferred embodiment, the invention provides for the technique of Molecular Indexing of DNA And Sequence (MIDAS).

In this embodiment, long stretches of previously unknown nucleic acid sequence are determined. Fragments to be sequenced are bar coded at fixed ends in the sequence, and the sequence determined at the opposite ends of the fragment to the bar code. The sequenced end of each fragment is at a random distance from the bar code so if all of the fragments are sized the end sequence information and the size information can be used to assemble the sequence in order of increasing distance from the bar code.

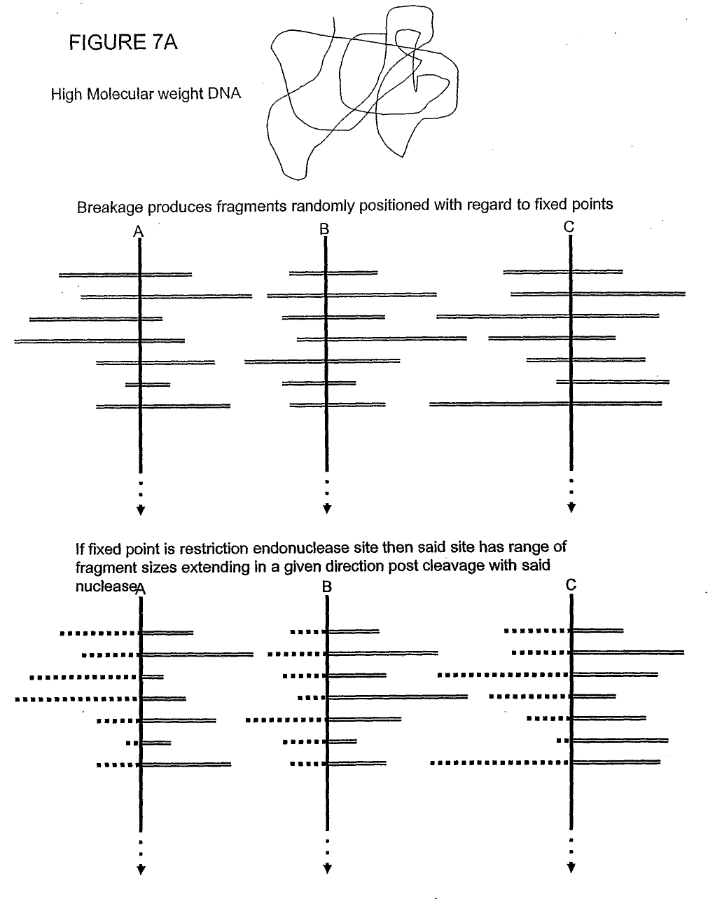

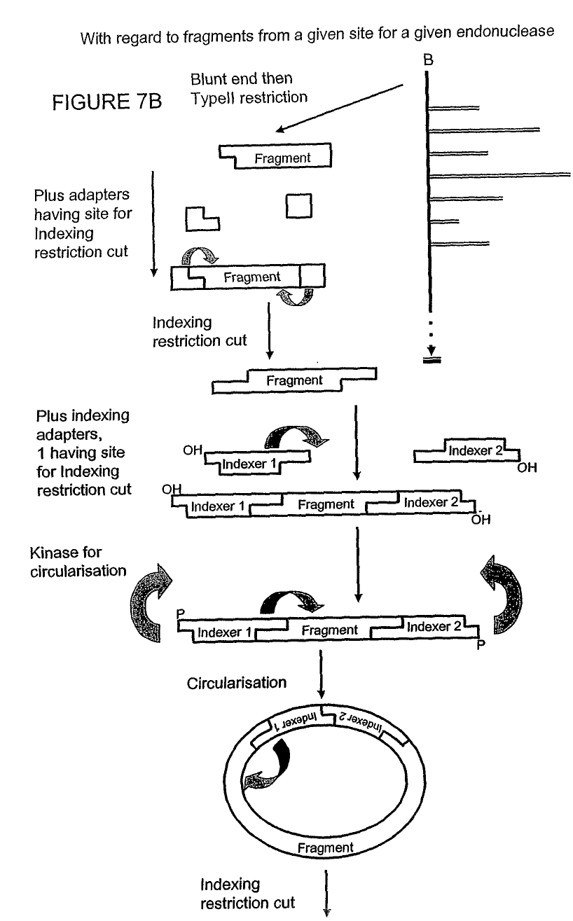

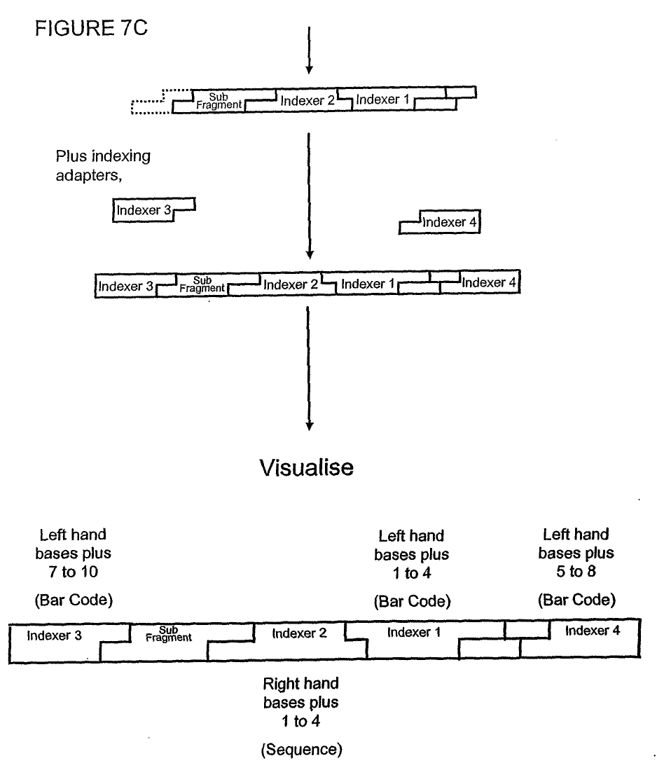

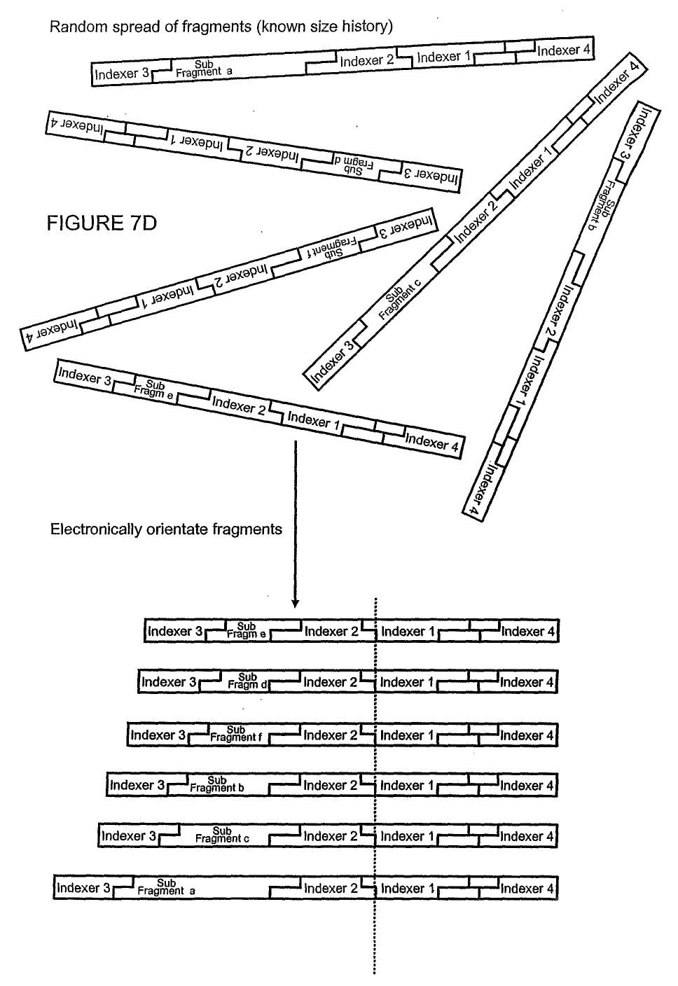

This is described in more detail in conjunction with FIG. 7. Considering a target nucleic acid population of sufficient original length for example whole chromosomes, it will be appreciated that most of the population is usually isolated as broken molecules having an average size dependent on the method of their isolation. This is illustrated diagrammatically in FIG. 7A where individual fragments are shown with respect to 3 different fixed points picked arbitrarily: A, B or C marked with a vertical arrow with respect to the fragments. The fixed points are found at a particular position with respect to the ends of a given fragment but at random distances with respect to the ends of the fragments as a whole population. It is possible to cleave at specific points within a fragment of nucleic acid for example by cutting DNA with a restriction endonuclease. A given cleavage point becomes a new end that is in common to all fragments that originally overlapped the fixed point cleavage site. The new fragments are shown with filled horizontal lines and as a whole form a population, each member having a specific end point, a certain distance from the fixed point determined by the cleavage. In practice there is a second set of fragments shown with horizontal dotted lines and these extend in the opposite direction from the point of cleavage.

Taking the fragments extending to the right in set B as an example, it is possible to define their orientation. In this case, orientation has been defined by ligating two different adapters to the fragments. One adapter has a cohesive end corresponding to the cohesive end produced at the fixed point by the restriction endonuclease and therefore ligates specifically to that end. The other adapter has a blunt end and ligates specifically to the ends of the fragment that were exposed by random shearing.

Having defined the orientation of the fragments it is possible for them to be indexed so that each end of a fragment receives an indexer that is different to the type found at its opposite end, shown in FIG. 7B as Indexer 1 and Indexer 2, respectively. The ends for indexing are produced by the action of a typeIIs restriction endonuclease or equivalent enzyme that cuts from the original adapters to expose unknown sequence a fixed distance into the ends of the original fragments. Appropriately labelled Indexers 1 and 2 determine the actual sequence by only ligating to fragment ends to which their ends are complementary. Indexers 1 and 2 are not initially allowed to ligate at their non indexing ends, in this case through having 5′ hydroxyl groups. Following the indexing, the non indexing ends of the indexers are allowed to join so that a circle is formed for example by first using polynucleotide kinase to phosphorylate the 5′ ends so that subsequent ligation is possible.

Indexer 1 has a site for a typeIIs restriction endonuclease so that the circle can be linearised by the action of this enzyme. Linearisation is performed to achieve two important consequences. Firstly, Indexers 1 and 2 remain joined on the original fragment and secondly a new position is exposed in the original fragment so that non redundant indexing can be performed on both of the exposed ends. A second round of indexing can therefore be performed using appropriately labelled Indexers 3 and 4 on each available end. The indexed fragments in the population can then be sized and then visualised or alternatively, visualised and then sized. In this example, the fragments have been randomly spread so that each can be seen as an individual molecule. Size can be determined by contour length but in preferred embodiments molecules will have first been sized before spreading.

The steps described result in two indexers on the end of each molecule and spreading and visualisation is performed so that these can be identified. Indexers represented here as 1, 3 and 4 have indexed the bases plus 1 to 4, plus 5 to 8 and plus 7′ to 10′, respectively. Together they can therefore be used as a code to identify fragments from the original fixed point. Indexer 2 determines the sequence at the opposite end of a fragment to the bar code. It is therefore a straightforward matter to determine the sequence from the fixed point by placing the fragments having a particular bar code in size order and reading their end sequence in turn.

Multiple sequences for example can be simultaneously determined in this way as long as it can be arranged for each useful fixed end to receive a different bar code and different bar codes can be distinguished. End sequences can then be associated with a particular bar code for sequence assembly for example A, B and C in FIG. 7A and each possible direction from the fixed points.

Further rounds of bar coding and/or end sequence determination are possible if Indexers 3 and 4 are ultimately allowed to ligate at their non indexing ends as above and one of them carries the site for a typeIIs endonuclease to allow cleavage of the new circles in a new part of the fragment for further indexing. Multiple rounds can be performed as often as required for the desired degree of information but for practical purposes one to two rounds are likely to be adequate.

It is not necessary to use the typeII restriction endonuclease to completely digest the original nucleic acid. Depending on the average size of the original fragments, this could result in many fragments having both ends produced by the enzyme rather than one end by shearing. It is preferable to perform a partial digestion so that most fragments are cleaved only once but in the population as a whole all possible cleavage sites are adequately represented.

This can be achieved either by limiting the period of the digestion or the concentration of enzyme. Some enzymes are sensitive to methylation in which case either a limited incorporation of modified nucleotides or the controlled use of site specific methylases will result in partial digestion.

The typeIIs endonuclease or equivalent should not be able to completely cleave the fragments other than from the site in the indexer as required by the method. The target fragments should therefore be similarly protected as above by appropriate modification.

It is not essential that the orientation of the fragments results from a blunt end and a cohesive end. Any distinguishable ends will suffice. For example the site for a typeIIs endonuclease could be situated by ligation of an adapter to allow a distinguishing cohesive end to be produced on the fragment ends. Nor do random fragments have to be produced by shearing. Any method that exposes all possible positions in a required sequence for reading will suffice. Ordered methods of reduction for example cyclical ligation and cutting could be used and have the advantage that ordered size reductions are possible in place of the sizing above. Pseudo random cleavage can also be produced by cutting partially with a restriction endonuclease having frequent sites. Fixed ends are produced in this case but these can be further processed to expose all possible required ends either by exonuclease digestion or by cyclical ligation and cutting. Exonucleolytic degradation of essentially intact nucleic acids is another way of exposing all possible nucleotides for sequence reading. Such degradation can also be controlled for example through the use of modified nucleotides. Endonucleases lacking specific recognition sequences can also be used to produce DNA that has essentially been cleaved at random places. It is well known that this can be achieved by DNAseI in the presence of Manganese ions. Appropriate dilution of the DNAseI allows the average rate of cleavage and therefore the average size of fragments obtained to be conveniently controlled.

It is not necessary that the bar code is made up from bases that were originally collinear, only that a sufficient number of bases are labelled to distinguish particular ends from each other. Nor is the circularisation essential. This is a convenient way using currently available materials to obtain sufficient information to distinguish particular ends in complex mixtures. If the mixture is less complex a simpler bar code requiring only one indexer per end may suffice. If means existed for exposing long cohesive ends for indexing at high fidelity then even the ends of a complex mixture could be indexed in a single step. This would however require a larger starting set of possible bar codes so it is advantageous to assemble bar codes by ligation of less complex units.

It is also possible to conduct a sort of fragments by indexing before the sequencing by indexing process above. This has the effect of sorting fragments into defined pools according to their end sequence. The complexity of each pool is therefore naturally less complex than the starting population so that fixed ends within each pool can be more easily distinguished. It also has the added benefit that sequence at the ends of the members of each pool are known as a consequence of the indexing and can be used as virtual information to add to the bar code or determined end sequence as appropriate.

It is not necessary that the sizes of the fragments are known precisely for sequence assembly. The visualisation of single molecules allows the frequency of end sequence to be counted in the fractions collected from a sized population. The frequency of each end sequence will initially rise and then fall through particular fractions in step with their relative size so it is only necessary to know their relative order rather than their absolute size. In fractions produced by electrophoresis for example, the end sequences of the shortest fragments will be more frequent in the earliest fractions to be replaced by the end sequences of increasingly longer fragments. Other methods of size fractionation, for example ion exchange or reverse phase chromatography, or using methodology employed in Chou et al., PNAS 1999; 96: 11-13 (incorporated herein by reference for all purposes) can be used with similar considerations to similar effect.

It is likely to be the case that sizing alone cannot be used to determine a precise order since there will be limits to the resolution of the technique used for sizing. However, Eulerian path assembly algorithm analysis can be used additionally to resolve ambiguities—see for example Pavel P et al. Proc Natl Acad Sci USA. 2001; 98: 9748-9753 (incorporated herein by reference for all purposes). In this analysis the actual order of bases is essentially anticipated by considering the possible order defined by the limits of what has been already observed. Thus, with sufficient lengths of end sequence the possible order of bases will be limited to the extent that precise order can be determined without necessarily knowing precise size—for example if 6 bases are determined on each fragment and 5′ AAAAC is observed then combinations of 5′ NAAAA must be immediately preceding and 5′ AAACN must be immediately following so the number of possibilities that could fit will be rare. This makes it relatively straightforward to determine the likely order. Sorting fragments by indexing as above is also helpful in this regard because it reduces the complexity of the fragment populations that are analysed and increases the amount of sequence known at the ends of the fragments. For example, a particular 4 base sequence will occur every 256 nucleotides whereas a 6 base sequence will occur on average every 4096 nucleotides. It is therefore far easier to place the occurrence of 6 base sequences in relative order than 4 base sequences because on average less resolution is required for the former.

It should be noted that this allows for the first time, sequences read lengths of significantly more than a kilobase to be assembled. For example, on average only 4 fragments per kilobase will be detected by a particular indexer recognising a particular 4 base end sequence i.e. 1 fragment per 250 base pairs. It is a relatively simple matter even with relatively low resolution techniques like agarose gel electrophoresis to resolve up to 10 kilobases, particular fragments of average spacing 256 bases. The precise order of fragments and therefore the sequence from which they arose can be deduced using the principles described above.

The above discussion is confined to multiple fixed points from a complex mixture. Should it be required to focus on a particular region of sequence then the same principles can be applied. One approach is to pick indexers to bar code a specific fixed points for reading through the region. This would likely involve sequencing some other regions by chance. It is also be possible to label fixed points of interest by hybridisation to a suitably labelled probe in which case the bar coding is not essential. Probes could be multiplexed for simultaneous reading of multiple known regions of interest.

The invention will now be further described and illustrated by means of the following examples. All oligonucleotides used in these examples were purchased as a custom synthesis by standard phosphoramidite chemistry from Eurogentec Ltd. The oligonucleotides were HPLC purified by the supplier before use.

### EXAMPLE 1

### Creation of and Lengthening of Single Stranded Overhanging Ends in Target ds DNA Molecule

The creation of and lengthening of single stranded overhanging ends in target ds DNA is achieved by way of a multi step procedure. The target DNA is first cut with a standard type II restriction endonuclease. In the present example this is achieved using Dpn II or Bgl II, restriction endonucleases which allow for the subsequent insertion of an N.Alw I site for recognition by the nicking enzyme N.Alw I. Following preparation of the lengthening adapter molecule, the adaptor is ligated to the target ds DNA. The product of the above noted ligation is then subject to nicking by exposure to N.Alw I. After nicking, the ligation product is cut with a further restriction endonuclease which, in the case of the present example is Avr II. The resulting target ds DNA containing lengthened single stranded overhanging ends can then be purified and visualised by agarose gel electrophoresis. In the present example, the lengthening adapter molecules are labelled so as to facilitate easy visualisation of the resulting products when resolved by electrophoresis.

The above summarised steps in the lengthening procedure as carried out in the present example are detailed separately below.

### Preparation of Target ds DNA.

Both Lambda DNA and Genomic DNA from various cell lines, are used as sources of target DNA and are isolated by procedures well characterised in the art.

Lambda DNA is N⁶-methyl adenine free, supplied by NEB. It is purified by phenol extraction and dialysed against 10 mM Tris-HCL (pH 8.0) and 1 mM EDTA.

Genomic DNA was also prepared from the following cell lines, H-tert from Clontech, MCF7 and KPL1, both from DKFZ, and U937 from the ATCC. The various cell lines were grown in tissue culture media as recommended and then harvested for DNA purification using the Genomic-tip system (Qiagen) as recommended by the manufacturer.

Lambda DNA is suitable for this example because its size and sequence are known so the fragments produced by particular restriction endonucleases and their behaviour in the process can easily be predicted and monitored.

Lambda and Genomic DNA were cut respectively with the restriction endonucleases Dpn II and Bgl II by incubation in the reaction mixtures outlined below:

For Lambda DNA the following reaction mixture was prepared:

| | |
|---|---|
| Lambda DNA (methyl adenine free; 0.5 μg/μl) | 60 μl (30 μg total) |
| ×10 Dpn II buffer (NEB) | 60 μl |
| Alpha H₂O (purified by reverse osmosis) | 480 μl |
| Dpn II (NEB; 10 units/μl) | 20 μl |
| Total | 620 μl |

For Genomic DNA, each of the following reaction mixtures were prepared:

| Reaction mix | A | B | C | D |
|---|---|---|---|---|
| *H-tert | 127 μl | — | — | — |
| *MCF7 | — | 94 μl | — | — |
| *KPL1 | — | — | 81 μl | — |
| *U937 | — | — | — | 23.6 μl |
| ×10 Buffer 3 (NEB) | 100 μl | 100 μl | 100 μl | 100 μl |
| Alpha H2O | 773 μl | 806 μl | 819 μl | 876.4 μl |
| Bgl II (NEB; 10 units/μl) | 20 μl | 20 μl | 20 μl | 20 μl |
| Total | 1020 μl | 1020 μl | 1020 μl | 1020 μl |

*It should be noted that the various volumes used for each of the different sources of Genomic DNA provide an amount of DNA to the reaction mixture of 20 μg.

All reaction mixtures above are incubated overnight at a temperature of 37° C. Following incubation, the resulting target DNA digest is purified by application of the reaction mixtures to Qiagen spin columns (QIAquick DNA clean up kit, as recommended by the manufacturer). For the purposes of purifying Lambda DNA digest from the reaction mixture, 3 Qiagen columns are used such that approximately 10 μg of DNA from the reaction mixture is applied to each column. In the case of Genomic DNA digest purification, the reaction mixture is purified using 2 Qiagen columns. Both Lambda and Genomic DNA digest are eluted from the columns using standard 0.15× elution buffer, EB (1×EB being 10 mM Tris-HCL, pH 8.0 and supplied by Qiagen). For each column, 60 μl of elution buffer is applied.

The assumed yield for both Lambda and Genomic DNA digest is 1 μg per 6 μl eluant. This equates to approximately 7 pmoles of ends/μg and 0.74 pmoles of ends/μg for Lambda and Genomic DNA digest respectively as calculated as follows:—

Lambda DNA has 116 DpnII sites. Each molecule of lambda DNA cut with DpnII therefore has 232 DpnII ends because each cut produces 2 ends. 1 μg of 1 kilobase of double-stranded DNA corresponds to 1.52 pmoles. Lambda is 48,502 kilobases in length so 1 μg is only 0.031 pmoles of lambda but these yield 0.031×232 pmoles of DpnII ends or 7.27 pmoles of DpnII ends per 1 μg of lambda DNA or in the case of the eluate per 6 μl of eluate.

Human genomic DNA is assumed to be 3.1 billion base pairs in length and have BglII sites on average at 4096 (4⁶) base pair intervals. This produces 756,836 BglII fragments and 1,513,672 BglII ends per human BglII digest. 1 μg of human genomic DNA is 4.9 e-⁷ pmoles which produce 4.9 e-⁷×1,513,672 pmoles of Bg1II ends or 0.74 pmoles per 1 μg of human DNA or in the case of the eluate per 6 μl of eluate.

### Preparation of Lengthening Adapter Molecule

The lengthening adapter molecule, in this example, the N.Alw I adapter, is specific for the end produced by the first enzyme used to cut the target ds DNA and provides a site for the nicking endonuclease N.Alw I. The two complementary strands of the N.Alw I adapter are prepared separately. The sequences of the separate strands are detailed below:

| | |
|---|---|
| AvDpL8b | 5′ GATCCTAGGTCTGAGATTCTCAGGATCT 3′ |
| FaAvDpU8b | 3′ GATCCAGACTCTAAGAGTCCTAGA 5′ |

The FaAvDpU8b adapter strand is labelled at its 5′ end with the fluorescent dye FAM (5/6 isomers added as phosphoramidite during synthesis to 5′ end). This label allows the fate of the adapter to be monitored during and/or following its use in the lengthening process. Phosphorylation of the 5′ end of AvDpL8b adapter strand, is brought about by incorporation of this adapter strand into a kinase reaction mixture. Conventionally, a standard kinase reaction mixture uses 200 pmoles target oligo per 50 μl reaction mixture. Accordingly, the following reaction mixture is prepared:

| | |
|---|---|
| Adapter AvDpL8b (50 pmol/μl) | 60 μl |
| ×10 T4 polynucleotide kinase buffer (NEB) | 75 μl |
| ATP (10 mM) | 75 μl |
| Alpha H2O | 540 μl |
| T4 Polynucleotide kinase (NEB; 10 units/μl) | 15 μl |
| Total | 765 μl |

It is most convenient to set up the above kinase reactions in 1.5 ml tubes as these can be more easily accommodated by a regular heating block.

Once prepared, the reaction mixture is incubated for a period of 3 hours at a temperature of 37° C. The reaction is then terminated by heat inactivation. This is achieved by incubating the reaction mixture at a temperature of 95° C. for 10 minutes.

Following heat inactivation, the kinased adapter oligonucleotide is then annealed to its labelled complementary strand, FaAvDpU8b. The oligonucleotides are mixed at a ratio of 1:1. The complementary strand is first prepared at a concentration equal to the concentration of the kinased oligonucleotide present in the reaction mix (i.e.; 200 pmol/50 μl or 4 pmol/μl). The oligonucleotides are then mixed in a 1:1 ratio giving a final concentration of 2 pmol/μl/oligonucleotide strand. The mix is heated to 95° C. for 5 minutes to remelt secondary structures. The strands are then annealed by incubating the mix at 65° C. for a period of 5 minutes. The resulting annealed adapter molecule is stored at −20° C. until use.

### Ligation of Adapter to Target DNA

Once prepared, the adaptor can be annealed and ligated to the end strands of the target DNA which has been digested as described above. When Lambda DNA digest is used as the target DNA, this is achieved by preparing the following basic reaction mixture:

| | | |
|---|---|---|
| Lambda DNA digest (1 μg/6 μl; i.e. | | 6 μl |
| 7 pmol/6 μl) | | |
| Adaptor molecule (560 pmol/80x excess) | | |
| X10 Ligase buffer (NEB) | | 7.5 μl |
| T4 DNA ligase (NEB; 400 CEL/ml) | | 0.75 μl |
| Alpha H₂O | up to a total of | 75 μl |

On the basis of the above and for the purposes of this example, the following 2 reaction mixtures are prepared:

| | Plus ligase | | | |
|---|---|---|---|---|
| | No adapter | | Plus ligase | |
| | (control) | | Plus adapter | |
| Lambda or Human digest | 12 | μl | 18 | μl |
| Adaptor | 0 | μl | 840 | μl |
| X10 T4 | 15 | μl | 102 | μl |
| Alpha H20 | 123 | μl | 520 | μl |
| T4 DNA Ligase | 1.5 | μl | 7.5 | μl |
| TOTAL | 150 | μl | 1480 | μl |

The above reaction mixtures are incubated overnight at a temperature of 16° C. Following incubation, the ligation products can be visualised by resolution on a 2% agarose gel, ran at 100 to 120 Volts for approximately two hours.

The gel is run unstained and visualised using a Typhoon or fluoroimager (fluorescence scanner) Amersham Pharmacia (GE Healthcare).

Visualisation is performed to show that the adapters have ligated to the DpnII fragments. For this purpose, three samples are loaded. A sample of DpnII digested lambda DNA together with the products of the two reaction mixtures indicated above. In the absence of adapters the lambda cannot be seen without staining. In the presence of adapters plus ligase the DpnII fragments can be seen because they are ligated to the FAM labelled strands. Their size is also seen to increase by the length of the added adapters when compared to the original material which can only be seen post staining with for example, ethidium bromide. In the presence of ligase but absence of adapters the lambda DpnII fragments ligate to each other. This appears as a higher molecular weight smear and can only be seen post staining. It shows that the fragments are capable of ligation.

Following ligation, excess unligated adapter molecules need to be removed before the ligated product can be exposed to the nicking endonuclease. In the present example, removal of excess unligated adapter molecules is achieved by ultrafiltration followed by application to Qiagen (QIAquick) spin columns, as detailed below.

Amicon Microcon 50, ultrafiltration columns having a size exclusion filter with a notional molecular weight limit of 50,000 daltons are used. The filter is in the middle of the column and the sample is loaded into the top half so that on centrifugation the water and low molecular weight material including the unused adapters pass through the filter leaving the adapter labelled fragments trapped above the filter where they become concentrated. Repeated loading and/or addition of buffer or water is carried out to further concentrate the fragments or to achieve increased purification. Once it is desired to collect the fragments, the columns are inverted onto a fresh tube and recentrifuged so that they fall in solution into the tube.

Typically 400 μl of fragment/adapter containing solution is loaded per column per run until all of the fragments have been loaded. The filters are then washed with 300 μl of water per run followed by a final wash of 150 μl of water and centrifugation before collection. The 150 μl wash minimises the residual solution before collection so that the concentration of purified fragments is maximised.

Centrifugation is at 1500 g for 12 minutes. The number of ultrafiltration columns is not critical because losses are not significant. As few as is convenient are used. Using fewer requires more loadings for large volumes of original ligation.

The ultrafiltered material corresponding to the original ligation is then purified using QIAquick columns as recommended by the manufacturer. Three rounds of purification are performed. In the first round, three columns are used, in the second round two columns are used and in the final round, one column is used.

60 μl of elution buffer are used per column so that 180 μl, 120 μl and 60 μl of eluate result after the first, second and third rounds, respectively. 1×EB is used in the first 2 rounds but EB is diluted 15:85 as above (to create a 0.15× solution) for the final elution.

The eluant obtained following removal of unused adaptor molecules, is then stored at −20° C. until use.

### Nicking of Ligated Adapter-Target DNA Product

Nicking of the ligated product purified as described above is achieved by incubating the product in the following reaction mixtures:

| | | |
|---|---|---|
| Ligated product sample | 1 μg (6 μl) | 10 μg (60 μl) |
| X10 buffer 2 (NEB) | 4 μl | 80 μl |
| Alpha H₂O | 40 μl | 660 μl |
| N. Alw. NI (NEB; | 2.5 μl | 25 μl |
| 400 CEL/μl) | | |

In addition to the above reaction mixture and as a control, a second reaction mixture is prepared identical to that described above except for the fact that it omits the nicking enzyme N.Alw I.

The above reaction mixture is incubated at 37° C. overnight. Following incubation, the nicked ligated product can then be digested in a manner as described below.

### Digestion of Ligated Product Post Nicking

The restriction endonuclease Avr II is used in this case to create an 8 base 3′ overhang at the end of the target DNA for ligation with indexing molecules. In order to cut the nicked ligated product, Avr II is added to the reaction mixture resulting from the above described nicking process. The enzyme is supplied at a stock concentration of 4 units per μl (NEB) and uses the same buffer as N.Alw I allowing it to be added directly to previous reaction mixtures. Of this stock concentration, the following volumes of Avr II are incorporated to the reaction mixtures:

| 1 μg reaction | 10 μg reaction | Length of |
|---|---|---|
| mixture | mixture | incubation |
| 2 μl | 20 μl | At least 3 hours |

If required then the reaction volume can be increased to avoid for example unfavourable concentrations of glycerol added with the enzyme. For example, if the total enzyme exceeds 10% of the total volume, extra buffer should be added. The concentration of the reaction buffer should however be maintained at 1× so 10× reaction buffer should be added in proportion to any water that is added.

As indicated above, Avr II is incubated with the ligated product at a temperature of 37° C. for a period of at least 3 hours.

However, as well known in the art prolonged incubation should be avoided so as to prevent unwanted side reactions, for example exonuclease activity.

Following restriction endonuclease digestion, the sample is then incubated at 75° C. for a period of 5 minutes or greater. After incubation, the DNA target molecules containing the 3′ overhanging ends, are purified by application to Qiagen columns as follows.

A final QIAquick purification is performed to remove residual adapter that is not ligated to or has been cut from the fragments. This adapter can be annealed to the fragments but not necessarily covalently joined. It is therefore helpful to first incubate the fragments at 75° C. to melt any annealed adapters from the fragments to maximise the likelihood of their removal from the fragments by QIAquick. Additional QIAquick purifications can be performed if gel analysis determines that residual adapter remains. The fragments are added immediately following heat treatment to at least 5 volumes of PB (proprietary buffer, Qiagen) and immediately loaded onto the QIAquick column. Up to 10 μg of fragments can be added per column.

Following application of the sample to the Qiagen columns and following washing with PB, the DNA fragments are then eluted from the column in 60 μl of 0.15×EB per column.

The resulting purified DNA fragments can be visualised on a 2% agarose gel following electrophoresis. The gel is run unstained at 100 to 120 volts for ca. 2 hours and visualised using a Typhoon or fluoroimager (fluorescence scanner) Amersham Pharmacia (GE Healthcare).

Visualisation is performed to confirm N.Alw I and Avr II digestion. Four samples are loaded; DpnII digested lambda, a sample of DpnII digested lambda DNA that has been previously adaptored, a sample of the N.Alw I digest and a sample of the Avr II digest. The latter is best taken after the final QIAquick purification. Only, the adaptored lambda should be visible in the unstained gel as a result of its FAM label in the adaptor. N.Alw I has the effect of nicking into the ends of the lambda fragments. This allows the FAM label to be removed by purification as described above (i.e. if the nicked strand of adapter plus a few bases of the lambda fragment(s) is melted from the remainder). Simply heating before loading the gel can be similarly informative. In the stained gel, little difference is seen in size between the N.Alw I digested material and the adaptored lambda. However, the sample that has been successfully digested with Avr II shows a size that is very similar to the unadaptored lambda DpnII digest, demonstrating appropriate removal of the adaptored ends.

The genomic digests are similarly processed as for the lambda digests.

### EXAMPLE 2

### Differential Labelling of Double Stranded Target DNA (Indexing)

Target DNA is incubated with two different indexing molecules in a ligation reaction mixture set forth below:

| | | |
|---|---|---|
| Lambda DNA from example 1 | 2.5 | μg |
| A index (1 pmol per μl) | 5 | μl |
| B index (16 pmol per μl) | 5 | μl |
| X10 Pfu (Strategene) | 10 | μl |
| Alpha H₂O | 50 | μl |
| PFU ligase (Strategene) | 1 | μl |
| Total | 100 | μl |

Thus, 0.5 μg of the lambda DpnII digest is used per 20 μl ligation. Two indexers are used; 1 μl per 20 μl reaction of the A indexer with ends GATCTCAC 3′ at 1 pmole per μl to select 3′ CTAGAGTG ends and 1 μl per 20 μl reaction of the B indexer with ends GATCTGNN 3′ at 16 pmoles per μl to select 3′ CTAGACAA, 3′ CTAGACAC, 3′ CTAGACAG, 3′ CTAGACAT, 3′ CTAGACCA, 3′ CTAGACCC, 3′ CTAGACCG, 3′ CTAGACCT, 3′ CTAGACGA, 3′ CTAGACGC, 3′ CTAGACGG, 3′ CTAGACGT, 3′ CTAGACTA, 3′ CTAGACTC, 3′ CTAGACTG, or 3′ CTAGACTT ends. The B indexer is used at a 16 fold higher concentration than the A indexer because it corresponds to 16 times more possible ends. A large excess of indexer to target ends is not used unlike the ligation in Example 1 because the fidelity of indexing reduces the instance of misligation which would be required for sample ends to join to each other.

The above reaction mixture is then subjected overnight, for at least 16 hours to continuous cycles as outlined below:

72° C. for 30 minutes
45° C. for 3 minutes
50° C. for 3 minutes
55° C. for 5 minutes

Alternatively, continuous incubation at 50° to 55° C. is performed for at least 16 hours, instead of the cycling reaction indicated above.

Following indexing the single stranded regions are filled in by carrying out the following T4 DNA polymerase reaction:

Per 20 μl Pfu DNA ligase reaction;
8 μl dNTPs @ 10 mM
8 μl×10 T4 DNA polymerase buffer (NEB)

### 44 μl H20

0.8 μl T4 DNA polymerase (NEB)

### 80 μl Total

The above reaction is performed at 12° C. for 30 minutes.

After the filling in reaction, the DNA is purified using QIAquick (Qiagen) as recommended by the supplier.

The purified material is then cut with the restriction endonuclease Bgl I. This reaction is performed using 10 units of Bgl II/μg of DNA per 20 μl reaction volume, over a period of 2 hours at 37° C.

The DNA is repurified as above and circularisation performed by ligation for 16 hours at 16° C. using 0.1 μl of T4 DNA ligase (NEB) per 10 μl of BpmI buffer (NEB) containing 1 mM ATP and 0.1 μg of DNA per 10 μl.

Following circularisation, purification of the circularised product is achieved by application to Qiagen columns as described above.

T4 DNA ligase was heat inactivated at 68° C. for 20 minutes. A BpmI site in the indexer A is used to cut 2 bases beyond the original indexed site. The BpmI cut is situated 2 bases beyond the original 4 indexed bases. Linearization is thus achieved by the addition of 4 units per μg of BpmI (NEB) at 37° C. for 2 hours or until reaction is complete. This has the effect of leaving the two joined indexers together on the same end of their fragment.

Following purification of the linearised ligation product using QIAquick as described above, a second round of indexing is commenced by incubation with an indexing molecule complimentary to possible bases left by the site of the type II endonuclease cut.

The indexing molecule used for the second round has complimentary strands whose sequences are outlined below.

| | |
|---|---|
| AvDpL8 | 5′ GATCCTAGGTCTGAGATTCTCAGGATCT 3′ |
| FaAvDpU8I | 5′ NNCTAGGATCCAGACTCTAAGAGTCCTAGA 3′ |

The second round of indexing uses the procedures described in Example 1 and continued in the proceeding part of Example 2 up to the point of ligating on the indexers with Pfu DNA ligase.

The ends have been produced by BpmI rather than DpnII. Therefore the first adapter ligated on by T4 DNA ligase is produced from AvDpL8 and FaAvDpU8I as described above. The 2 bases NN of FaAvDpU81 are selected as appropriate for the possible ends exposed.

The final indexer ends AATG 3′ corresponding to the next bases of the fragment. For visualisation on the MegaBACE (GE Healthcare) the 5′ end is HEX TAATGATC to allow detection. The MegaBACE is used with a Genotyping set up as recommended by the supplier. Size markers are 0.2 μl per capillary of ET ROX 900 (GE Healthcare). Prior to loading samples are desalted by ultrafiltration as previously described and made up to 0.1% Tween 20. Intermediate products of the procedure can be analysed in separate capillaries to monitor the process. DNA corresponding to 0.5 μg of original starting material is more than adequate for detection.

For visualisation of single molecules by epifluorescence a Zeiss Axioskop or a motorised Zeiss Axiolmager Z.1 fitted in either case with an AxioCam HRM Rev 2.0 Mono digital camera coupled to an AxioVision4 P4 3.0 GHz system. Methods were adapted from Optical Mapping procedures (Jing J et al, Proc Natl Acad Sci USA, 1998 Jul. 7; 95(14):8046-51, incorporated herein by reference, for all purposes). Modifications to the methods were according to the procedures deduced for hydrophobic molecules as described in Example 9.

In the present example, the indexing molecules were derived from phiX174 DNA. 0.5 μg of single-stranded virion DNA (NEB) was heated in 25 μl of BssHII buffer (NEB) to 95° C. for 2 minutes and annealed to an oligonucleotide extending from 16 bases to the side of the BssHII site, through the site and the PstI site to end 16 bases beyond the PstI site. The DNA was linearised by 5 units of BssHII for 1.5 hours at 50° C. The reaction was heated to 72° C. for 5 minutes and a second lot of enzyme added. The incubation continued at 50° C. for a further 1.5 hours. The reaction was adjusted to Taq DNA polymerase buffer (NEB) except that dTTP was substituted by aminoallyl dUTP (InVitrogen—formerly Molecular Probes) and the single stranded regions filled in using 2.5 units of Taq DNA polymerase under non strand displacing conditions (50° C.). DNA was purified (QIAquick) and ligated using T4 DNA ligase (NEB) to the appropriate indexer above which lacked the BglII site and had been rendered partially double-stranded by a complementary oligonucleotide to leave a BssHII compatible cohesive end. The DNA was purified as above, cut with PstI (NEB) and ligated to a blunt to PstI cohesive ended adaptor having a BglII site. DNA was repurified as above and labelled with an Alexofluor dye of choice using an Ares Alexofluor system according to the suppliers conditions (InVitrogen—formally Molecular Probes). The Alexofluor dyes substituted for the intercalating dyes described in Jing et al above.

### EXAMPLE 3

### ØX174 by Midas (FIG. 7)

In the following Example, unless otherwise stated, after each step of the various processes, DNA was routinely purified by ionic exchange chromatography according to the manufacturers instructions (QIAquick, Qiagen). Oligonucleotides were synthesised and HPLC purified by Eurogentec.

Genomic DNA from the bacteriophage ØX174 is analysed here as an example. It has the virtue that it is relatively simple and lacks sites for the typeIIs restriction endonuclease AcuI which is used during the process. There is therefore no need to block AcuI sites by methylation or other forms of modification. The indexers are also made from ØX174. Except for the lack of the AcuI this is only relevant in that single-stranded ØX174 DNA can be obtained easily for labelling and that its size is convenient for visualisation.

### Preparation of Target

### Size Reduction

The process takes advantage of the fact that DNA breaks at random during purification. The average read length is equal to the average size of the fragments obtained. Fragments having a higher average size allow longer possible read lengths but require a greater mass of fragments per base read since only the bases at the ends of the fragments are read. It is therefore desirable to shorten fragments by further random breakage should their average length be too long. This is commonly achieved by sonication (to obtain short fragments) or repeated drawing of the DNA solution through a suitably sized syringe needle if less severe breakage is required (narrower gauge needles achieve greater shearing). We have also used DNAseI in the presence of manganese to fragment DNA. Typically, 0.2 to 0.4 ug of DNA were used per 15 ul reaction containing: 50 mM Tris HCl pH 7.6, 10 mM Manganese Chloride 0.1 mg/ml BSA (NEB): 0.5 to 0.05 units of DNAseI (NEB). Reactions were performed at 37° C. for 20 minutes and immediately purified as standard above. The origin of the DNA is not significant for the method only that conditions for breakage or a suitable dilution of enzyme is found by experiment to achieve the required extent of size reduction.

### End Repair

Shearing or other forms of breakage commonly leave ends that are not blunt and therefore do not serve as a good substrate to which to ligate blunt ended adapters required for the end from which sequence will be determined. Where blunt ends were required we routinely incubated 1 ug of DNA per unit of T4 DNA polymerase (NEB) at 12° C. for 30 minutes in a volume of 30 ul containing 100 mM equimolar mixture of the 4 deoxynucleotides dATP, dCTP, dGTP and dTTP in buffer 2 (NEB).

### Methylation Protection

The process uses the action of restriction endonucleases. There may be one or more sites for these endonucleases in between the two ends of fragments of interest. If these sites are cut thus separating the ends of interest onto separate molecules the required information may be prevented from being provided i.e. which end sequence goes with which coded end. Such cutting can be avoided by appropriate methylation of the target sites. The combination of enzymes that we have found to be useful include the nicking endonuclease N.AlwI whose sites were protected by dam methylase, AvaI whose sites were protected by M. SssI methylase and the Type IIG endonuclease Acu I, a polypeptide having both endonuclease and cognate methylation protection activities together (all NEB). In the case of the latter, it was convenient to switch between the two activities by inclusion or exclusion of the divalent cation, in this case magnesium and S-adenosyl methionine. Magnesium is required for the endonuclease and if this activity is not required then magnesium can either be left out of the reaction or chelated in the reaction by EDTA (15 mM final in our reactions). 160 umolar final of S-adenosylmethionine was routinely used. This needed to be tailored to the target otherwise situations could arise where insufficient mass of S-adenosylmethionine was added to protect all possible sites. Incubation was at 37° C. for 16 hours. All three methylases could be used at once in which case the buffer for dam methylase (NEB) served as a universal buffer. Some enzymes are known to cut infrequently and if used as the double stranded endonuclease it is not essential for their sites in the target to be protected. SalI and SapI for example produce fragments of average size 83,000 and 14,000 bases respectively in the human and would show different site selection biases due to the nature of their different recognition sequences. Note that cutting originally with an enzyme that leaves a 5′ gatc overhang (the recognition site for dam methylase) is the easiest way to leave an end that can be made compatible with the nicking endonuclease N.AlwI. The latter is sensitive to dam methylase so care should be taken to ensure that created sites are not blocked. EcoRV sites for example can be converted to N.AlwI sites by adapter ligation and the former are not subject to dam methylation.

### Generation and Lengthening of Cohesive Ends

For use as a target double-stranded ØX174 DNA was digested with the restriction endonuclease HinfI. HinfI cuts at the sequence gantc i.e.

(5′ G\hat{ }ANTC 3′
3′ CTNA\hat{ }G 5′)

and as a consequence several fragments having a defined orientation with respect to their end sequence are produced. DNA was purified using a Qiaquick purification system as recommended by the supplier and ligated to two sets of adapters simultaneously. The first set of adapters has a 5′ ACT cohesive end and c as the first internal base so that on ligation to the complementary sequence 5′ AGT an NBstNB1 site is created. The second set of adapters has a 5′ ADT cohesive end (where D=A, G or T) and serve as blockers by ligating to all other possible HinfI ends so that they are prevented from ligating to each other and so that they do not form an NBstNB1 restriction site with the adapters. The first set of adapters has an XbaI site juxtaposed to the bases forming the NBstNB1 site so that on digestion with NBstNB1 followed by XbaI an 8 base 3′ cohesive end is produced where the first 4 bases from the 3′ end are known and the next 4 bases originate from the target fragment immediately beyond the original HinfI site. Set 1 adapters were also labelled at their 5′ end with FAM so that their fate could be monitored. The sequence of the adapters were therefore as follows:

| | |
|---|---|
| Set 1 | |
| HfXbL8b: | 5′ ACTCTAGATCTGAGATTCTCAGGATCT |
| FaHfXbU8b: | 3′ GATCTAGACTCTAAGAGTCCTAGA-6-FAM |
| Set 2 | |
| Hf920L: | 5′ ADTGCACCAAAGTAACCCTG |
| | D = A, G, T mix |
| Hf20U: | 3′ CGTGGTTTCATTGGGAC |

An unknown target DNA population would be ligated to adapters in the ratio of set1:set2 of 1:3. However as the sequence of ØX174 DNA was known allowance could be made for the actual ends. There are HinfI fragments, i.e. 42 ends, of which 10 ends have an “ACT” complementary overhang and 32 will have “ADT” complimentary overhang. An appropriate ration is therefore 10 of Set1 to 32 of Set2.

### Synthesis of Lengthening Adapters

Each adapter is synthesised by standard chemistry (Eurgentec) as two unphosphorylated oligonucleotides and then 5′ phosphates added for ligation by T4 polynucleotide kinase. A standard kinase reaction uses 200 pmoles of target oligonucleotide per 50 ul reaction. Oligonucleotides were therefore made up to a convenient concentration, e.g. 50 pmoles/ul in water so that 200 pmoles (4 ul) can easily be added to the reaction. In Set1 and Set 2 the phosphorylated oligonucleotides were HfXbL8b and Hf920L respectively.

### Kinase Reaction:

75 ul ×10 T4 polynucleotide kinase buffer

### 75 ul ATP 10 mM

60 ul oligonucleotide @ 50 pmoles/ul
540 ul alpha H₂O
15 ul T4 polynucleotide kinase 10 u/ul (NEB)

### 765 ul Total

Incubation was performed at 37° C. for 3 hr (waterbath). Heat inactivation of the polynucleotide kinase was performed at 95° C. for 10 mins.

The kinased oligonucleotides were annealed to their complementary strands to produce the adapters. Complimentary strands were diluted in alpha H₂O to the same concentration as the kinased oligonucleotides (200 pmoles per 50 ul or 4 pmoles/ul) i.e. 60 ul complimentary oligo @ 50 pmol/ul+700 ul water.

Complementary oligonucleotides were mixed 1:1 (380 ul+380 ul in 1.5 ml tube) to give a final concentration of 2 pmoles/ul of double-stranded oligonucleotides, heated to 95° C. for 5 mins to reduce secondary structures and then annealed at 65° C. for 5 mins. Storage was at −20° C. if required.

### Ligation of Lengthening Adapters to Target DNA

Adapters were ligated at an 80 fold molar excess over the HinfI fragments to ensure that the majority of HinfI ends were adaptored. T4 DNA ligase works in the PNK buffer, therefore the final reaction was simply adjusted to account for the other added materials as follows:

80 ul (10 ug) HinfI digested ØX174 DNA
1144 ul Set1 adapters @ 2 pmol/ul
3661 ul Set2 adapters ds @ 2 pmol/ul
280 ul ×10 T4 ligase buffer (NEB)
9 ul alpha H₂O
26 ul T4 DNA ligase (NEB)
5200 ul total volume

Incubation was performed @ 16° C. for 16 hours.

Control reactions were set up similarly except that in the no adapters control the adapters were replaced by 0.5×PNK buffer and in the no ligase control the T4 DNA ligase was omitted.

Ligations were purified by ultrafiltration and then three successive Qiaquick purifications, the latter as recommended by the supplier except the elution buffer EB was diluted to 15/100 with alpha H₂O.

Ultrafilters were prewet with 400 ul alpha H₂O and drained by centrifugation at 1500×g for 12 mins or until less than 40 ul remained. Ligation reactions were applied to the ultrafilters 350 ul at a time (2.5 ug per filter total) with centrifugation as above between additions until all the reaction had been loaded and concentrated. A wash with 400 ul of alpha H₂O was also performed followed by a final wash of 150 ul of alpha H₂O to reduce the volume to less than 40 ul. Filters were inverted to a fresh tube a recentrifuged to collect the samples. 3 Qiaquick columns were used for the first round, 2 for the second round and 1 for the final round of Quiaquick purifications. If yellow colouration indicated that residual FAM labelled adapter remained then further rounds of purification were performed until clear. Samples could be stored at −20° C. until required.

### Preparation of Ends for Indexing

Ends for indexing were produced on the fragments by the action of NBstNBI followed by XbaI and then purification as follows.

˜50 ul (9 ug) HinfI digested and adaptored ØX174 DNA
21.5 ul NBstNB1 @ 10 u/ul (NEB)
61.5 ul ×10 buffer 3 (NEB)
477 ul alpha H₂O
Final volume 610 ul

Reactions were incubated for 16 hours @ 55° C.

XbaI does not work well in buffer 3 so the reaction volume was diluted as follows:

576 ul NBstNB1 reaction from above
230 ul ×10 buffer 2 (NEB)
1482 ul alpha H₂O

### 16 ul XbaI (NEB)

Final volume 2304 ul

Incubation was performed for 3 hours @ 37° C.

Purification was by Quiaquick (<10 ug per column) as recommended by the supplier except that samples were incubated at 75° C. for 5 minutes before immediately adding to PB and the columns. Quiaquick purification in this way removes the short oligonucleotide released by NBstNB1 and can be repeated if necessary. 0.5 ug samples were analysed by 2% unstained agarose gel electrophoresis and visualised using a Typhoon Fluoroimager (GE Healthcare) to confirm that each step had occurred correctly and the short products from the adapters had been removed. The presence of DNA following removal of the FAM labelled oligonucleotide was shown by staining with cyber green or ethidium bromide. In the absence of adapters the HinfI fragments formed a higher molecular weight smear by ligation to each other and in the absence of ligase no adaptoring as shown by the increase in the sizes of the HinfI fragments of the fragments occurred. NBstNB1 removed the FAM label and XbaI reduced the size of the fragments closer to their original size.

Successfully treated fragments were now ready for the sequence determination steps.

### Indexing

### Preparation of Indexer Molecules

Indexer molecules were first prepared from single stranded, ØX174 virion DNA which was used as a template for the synthesis of a second strand of DNA incorporating aminoallyl dUTP. The latter was directly labelled using ARES DNA labelling kits (Molecular Probes) with any preferred combination of the Alexa Fluor dyes. The primer for the second strand synthesis was

phiXBssH11-Pst1rc
TGGAAGCGATAAAACTCTGCAGGTTGGATACGCCAATCATTTTTATCGAA
GCGCGCATAAATTTGAGCAGAT

which overlaps both the BssHII and PstI recognition sites of ØX174 so that these can be cut following incorporation of the aminoallyl dUTP without fear of interference. Synthesis was performed as follows:
10 ul virion DNA 1 ug
320 ul alpha H₂O
50 ul ×10 Thermopol buffer (NEB)
60 ul aminoallyl dUTP (0.5 mM) (Molecular Probes)
10 ul dTTP (0.5 mM) (Amersham Pharmacia—now GE Healthcare)
40 ul d(GAC)TP (0.5 mM) (Amersham Pharmacia—now GE Healthcare)
10 ul phiXBssH11-Pst1rc @ 6 pm0l/ul in alpha H₂O
5 ul Taq DNA polymerase (NEB)

Final volume 500 ul used in 10×50 ul aliquots. Incubation was performed @ 50° C. for 2 hours and DNA purified by 2 Quiaquick columns (10 ug per column) as recommended by the supplier. Purified DNA was digested by PstI and BssHII as follows.

60 ul ØX174 aminoallyl dUTP incorporated DNA (10 ug above)
30 ul ×10 buffer 3
200 ul alpha H₂O

### 10 ul Pst1

Final volume of 300 ul

Incubation was performed @ 37° C. for 2 hours.

20 ul of BssH11 were added and incubation continued at 37° C. for 2 hours. 0.5 ug (15 ul) samples were analysed by 1% agarose gel electrophoresis to confirm digestion. A parallel control reaction was set up to show that BssHII also digests the modified DNA.

DNA was purified by Qiaquick as recommended by the supplier.

The PstI end was modified for use as an indexer and the BssHII end to allow circularisation. Any one of a family of oligonucleotides are used for the indexing end. The end for circularisation is designed not to be self complementary but to allow ligation to other indexers having a complementary end so that circles can only be formed from fragments having received two different types of indexer—the equivalent of 1 and 2 in FIG. 7. Modification of the aminoallyl dUTP ØX174 was by ligation of appropriate oligonucleotide adapters:

The Pst1 (indexing) end uses

| | |
|---|---|
| pxI1Hf/AAGT | 5′AACCCACATCTACAGACCCTGAAGAAACAGTCAAGT |

and the partially complementary strand

| | |
|---|---|
| pxI1Hf/U | 3′ ACGTTTGGGTGTAG |

and is the equivalent of Indexer 1 in FIG. 7 (the typeIIs site is for AcuI 5′CTGAAG 16/14);
or

| | |
|---|---|
| pxI2Hf/GCCA | 5′ AACCCACATCTACAGACCAAGCTGAAACAGTCGCCA |

and the same partially complementary strand. Note that this lacks the typeIIs site and is equivalent to Indexer 2 in the figure.

Oligonucleotides for use as indexers of the form (pxI1Hf/NNNN and pxI2Hf/NNNN) require phosphorylation by T4 polynucleotide kinase as described above.

The BssHII (circularising) end uses:

| | |
|---|---|
| Either | |
| pxCIRC/GACT | 5′ GACTGAAGTGATCTCCCT |
| or | |
| pxCIRC/AGTC | 5′ AGTCGAAGTGATCTCCCT |

with the partially complementary strand:

| | |
|---|---|
| pxCIRC/U | 3′ CTTCACTAGAGGGAGCGC. |

pxCIRC/U require a 5′ phosphate addition for ligation as above. Formation and use of the adapters is as described above except that the large size of ØX174 allows the unused adapters to be easily purified away by an S200 spin gel chromatography spin column (GE HealthCare) used as suggested by the supplier and in place of the ultrafilters. A single Qiaquick purification (as above) followed the spin column. Note that one end of ØX174 receives the pxI2Hf/NNNN based indexing adapter and the other the pxCIRC circularising adapter. Only one combination of indexing oligonucleotide and circularising adapter are used per indexer.

Indexers that were not able to circularise were also used. These were specific for the fragment ends that were not in this case of interest i.e.:

| | |
|---|---|
| pxI1Hf/TACT | 5′ AACCCACATCTACAGACCAAGCTGAAACAGTCTACT |
| pxI2Hf/ATTT | 5′ AACCCACATCTACAGACCAAGCTGAAACAGTCATTT |
| pxI2Hf/TCAT | 5′ AACCCACATCTACAGACCAAGCTGAAACAGTCTCAT |
| pxI2Hf/TTCT | 5′ AACCCACATCTACAGACCAAGCTGAAACAGTCTTCT |
| pxI2Hf/AATA | 5′ AACCCACATCTACAGACCAAGCTGAAACAGTCAATA |
| pxI2Hf/CGAT | 5′ AACCCACATCTACAGACCAAGCTGAAACAGTCCGAT |
| pxI2Hf/CACA | 5′ AACCCACATCTACAGACCAAGCTGAAACAGTCCACA |
| pxI2Hf/GAAA | 5′ AACCCACATCTACAGACCAAGCTGAAACAGTCGAAA |

These were non ØX174 and non labelled but otherwise the same as the indexing adapters.

Purified indexers were labelled with Alexofluor dyes using the Ares labelling kits as recommended by the supplier (Molecular Probes). Although Alexa Fluor dyes are quite stable, they are subject to light degradation, therefore for optimum fluorescence the indexer DNA was labelled as late in the process as possible and stored frozen in the dark. Repeated cycles of freeze/thawing were avoided.

### Ligation of Indexers

The ØX174 target DNA prepared initially for indexing was now indexed using the labelled indexers as follows:

1.4 pmoles of ØX174 target DNA ends (0.5 ug)
0.4 pmoles of Indexer 1 (pxI1Hf/AAGT based with pxCIRC/GACT)
0.4 pmoles of Indexer 2 (pxI1Hf/GCCA based with pxCIRC/AGTC)
3.2 pmoles of non circularising indexer (equimolar each) mix
2 ul X10 Pfu ligase buffer (Stratagene)
Alpha H₂O to a final volume of 20 ul
0.2 ul of Pfu DNA ligase (Stratagene)

Incubation was performed for 16 hours using continuous cycles of:
72° C. for 30 seconds
45° C. for 3 minutes
50° C. for 3 minutes
55° C. for 5 minutes

Reactions were purified (Qiaquick—Qiagen as recommended by the supplier) and the ends of the indexers phosphorylated using 1 ul of T4 polynucleotide kinase (NEB) in 40 ul of T4 DNA ligase buffer (NEB) at 37° C. for 1 hour. 0.4 ul of T4 DNA ligase (NEB) were added and the fragments allowed to circularise for 3 hours at 16° C.

The reaction was adjusted to 100 ul of T4 DNA polymerase buffer (NEB) and 1 ul of T4 DNA polymerase added to fill the single-stranded region of the indexers for 30 minutes at 12° C. Taq DNA polymerase (NEB) in its own buffer could also be used at 50° C. DNA was immediately purified from the reaction by Qiaquick (Qiagen) as recommended by the supplier.

### Cutting Back, Lengthening of Ends and Re-Indexing

The purified DNA was digested to completion by 10 units of AcuI (NEB) for 3 hours at 37° C. in 40 ul of the suppliers buffer.

DNA was purified and reindexed as above except that the adapters used to add the NBstNBI site were:

| | |
|---|---|
| 3nnXbL8b: | 5′GACTCTAGATCTGAGATTCTCAGGATCT |
| Fa3nnXbU8b: | NNCTGAGATCTAGACTCTAAGAGTCCTAGA-6-FAM 5′ |

And the indexers corresponding to 3 and 4 in the figure were not processed beyond the Pfu DNA ligation.

Indexer 3 was derived from:

| | |
|---|---|
| pxI2Hf/AGTA | 5′ AACCCACATCTACAGACCAAGCTGAAACAGTCAGTA |

Indexer 4 was derived from:

| | |
|---|---|
| pxI2Hf/CTGA | 5′ AACCCACATCTACAGACCAAGCTGAAACAGTCCTGA |

Reaction products were analysed and purified by 0.6% pulse field agarose gel electrophoresis and visualised using the Typhoon Fluoroimager (GE Healthcare) to detect the reaction products corresponding to fully and partially indexed products. Fully indexed products (>20 kb) were excised from the gel, purified using the QIAEX II Gel Extraction System (Qiagen) according to the supplier and visualised by epifluorescence using a Zeiss Axioskop or a motorised Zeiss Axiolmager Z.1 fitted with an AxioCam HRM Rev 2.0 Mono digital camera coupled to an AxioVision4 P4 3.0 GHz system. Methods were adapted from Optical Mapping procedures: Proc Natl Acad Sci USA. 1998 Jul. 7; 95(14):8046-51, Automated high resolution optical mapping using arrayed, fluid-fixed DNA molecules, (Jing J, Reed J, Huang J, Hu X, Clarke V, Edington J, Housman D, Anantharaman T S, Huff E J, Mishra B, Porter B, Shenker A, Wolfson E, Hiort C, Kantor R, Aston C, Schwartz D C, incorporated herein by reference for all purposes). Modifications to the methods were according to the procedures deduced for hydrophobic molecules as described in Example 9.

In order to achieve the >20 kb Indexed target the fate of the ends were as follows:

The Acu1 site is present in Indexer 1 only and circularisation occurred followed by digestion with AcuI at the point shown below.

Indexer 1
AcuI

| | |
|---|---|
| 5′CTACAGACCCTGAAGAAACAGTCAAGT5678910NNNNNNN | Target |
| 3′GATGTCTGGGACTTCTTTGTCAGTTCA5678910NNNNNNN | |

The second set of adapters are ligated on using T4 ligase and then the product is digested with NBstNBI and XbaI, as previously.

```
Indexer 1
                              XbaI
TC12345678GACTCTAGATCTGAGATTCTCAGG
AG12345678CTGAGATCTAGACTCTAAGAGTCC
Adapter 2
                      NBstNB1
-----TC12345678GACT CTAGATTTCTCAGG
-----AG12345678CTGAGATC TAAAGAGTCC
```

The 12 base pair oligonucleotide and short remainder of adapter2 are removed by the Qiaquick clean-up after the 75° C. incubation.

Pfu DNA ligase then ligates on the final indexers.

-----TC12345678GATC GATGTGGGTT
-----AG12345678CTAGCAAAGTCGAACCAGACATCTACACCCAA
Indexer 4

Similarly at the target (non-barcode end) after digestion with NBstNBI, XbaI and purification, followed by pfu DNA ligation with Indexer 3:

CCTGAGAATCTCAGATCTAGAGTC789101112------Adapter 2
GGACTCTTAGAGTCTAGATCTCAG789101112------Target

### Becomes

AACCCACATCTACAGACCCTGAAGAAACAGTC789101112----
Adaptor 3
TTGGGTGTAGA TCAG789101112----
Target

### EXAMPLE 4

### Choice of Ligase and Conditions for Ligation

Pfu DNA ligase (Stratagene) may be used at 50° C. for indexing reactions. However, many ligases are known and it is known that they can have different optimum conditions. Typically, the accumulation of pyrophosphate at the 5′ end of fragments to be joined can prevent ligation if it was not initially successful. Manganese can be a better source of divalent cations than magnesium in some cases (see for example Liu et al, Nucleic Acids Res. 2004; 32: 4503-4511, Tong et al, Nucleic Acids Res. 2000, 28, 1447-1454, Tong et al, Nucleic Acids Res. 1999, 27, 788-794). The following describes a screen for preferred ligases and their conditions of use.

### Generation of Target DNA Ends

F X174 RF DNA (NEB) cut to completion with HinfI was used as the target. HinfI is a convenient enzyme to use because one of its recognition sites 5′ G^{V}AGTC where ^{V} is the point of cleavage can be can be adapted to form the recognition site for the nicking. endonuclease N.BstNBI.

HinfI digestion was as follows:

### 30 μg F X174 (RF1 NEB)

30 μl HinfI (10 units/μl NEB)
60 μl ×10 buffer 2 (NEB)
480 μl water

Incubation was performed at 37° C. for 3 hours and standard purification performed.

### Preparation of Adapters

Two types of adapter were used, one for generation of N.BstNBI ends and one for the remainder:

| | |
|---|---|
| HfXbL8b: | 5′ ACTCTAGATCTGAGATTCTCAGGA |
| With | |
| FAMHfXbU8b | 3′ GATCTAGACTCTAAGAGTCCTAGA (5′FAM) |

which recreates the N.BstNBI site at 5′ AGTC/G ends and places an XbaI site next to it; and

| | |
|---|---|
| Hf920: | 5′ A(AGT)TGCACCAAAGTACCCT |
| With | |
| TAMHf20U | 3′ CGTGGTTTCATTGGGAC (5′TAMRA) |

which labels all the other possible ends with TAMRA i.e. 5′ CGTC/G, 5′ GGTC/G and 5′ TGTC/G.

HfXbL8b and Hf920 were kinased to add a 5′ phosphate as follows.

### 75 μl ATP 10 mM

75 μl X10PNK buffer
60 μl oligo 50 pmol/μl
540 μl water
150 units T4 Polynucleotide kinase (NEB).

Incubation was at 37° C. for 3 hours then heat inactivation was performed at 95° C. for 10 mins (no purification was used).

The complementary strands were added in a volume of water equal to the original reaction volume to a final concentration of 2 pmolar each and annealed at 60° C. after first denaturing at 95° C. for 5 minutes.

### Ligation

The adapters were added to the target as follows:

### 60 μl f X174/HinfI DNA (10 μg)

1259 μl FAMHfXbU8b/HfXbL8b (ds) 2 pmol/μl
944 μl Hf920TAM/Hf20u (ds) 2 pmol/μl
944 μl Hf920/TAM Hf20u (ds) 2 pmol/μl
1888 μl Hf920/Hf20u (ds) 2 pmol/μl
298.5 μl ×10 T4 DNA ligase buffer (NEB)
80 μl water
25 μl T4 DNA ligase (400 units/ul NEB)
and incubation performed at 16° C. for 16 hrs.

Adaptered fragments were purified by washing twice with 400 ul of water and then once with 150 ul of water in 4 ultrafilters (Microcon 50, Amicon) centrifuged at 1500×g for 12 minutes between each wash followed by three successive rounds of ion exchange chromatography (QIAquick, Qiagen) through 3 then 2 then 1 column. Elution was in 60 μl EB supplied.

### Cohesive end Labelling

Adaptered material was nicked by N.BstNBI as follows:

### 52 μl f X174/HinfI A/M (9 μg)

19 μl NBstNBI (10 units/μl NEB)
38 μl ×10 NBstNBI buffer (NEB)
271 μl water

Incubation was performed at 55° C. for 16 hrs.

The nicked material was then cut by XbaI.

370 μl of digest above
148 μl ×10 buffer 2 (NEB)
9 μl XbaI (20 units/μl NEB)
953 μl water

Incubate 37° C. for 3 hrs.

The short fragments of adapter plus end sequence produced by the endonucleases were melted from the target by heating at 75° C. for 10 mins, and immediately added to 5 volumes of PB buffer (Qiagen) for immediate (to avoid reannealing) purification by of ion exchange chromatography (QIAquick, Qiagen) using 3 columns.

Purification was repeated as before except that 1 column was used. Elution was with 60 ul EB buffer supplied and the new material referred to as Target f X174/HinfI DNA.

### Test Indexing Reactions

Indexing ligations were performed as follows:

### 3 μl Target f X174/HinfI DNA

2 μl ×10 ligase buffer (as appropriate—see table 2)
Up to 1.2 μl or 0.6 pmoles of each indexer
water to 20 ul
0.4 units of ligase (as appropriate—see table 2)

Incubation was performed at a selection of temperatures—see table for 16 hrs and the reactions analysed using a MegaBACE (GE Healthcare) fluorescence, capillary electrophoresis system after purification by washing 400 ul of water twice and then 150 ul of water with ultrafiltration at 1500×g for 12 minutes between washes.

TABLE 2

| | | Amount of | |
|---|---|---|---|
| Ligase | Buffer | ligase | Temperatures |
| Pfu ligase | X10 pfu ligase buffer | 0.1 μl = 0.4u | 37° C., 45° C. |
| | Mg2+ | | |
| Pfu ligase | X10 pfu ligase buffer | 0.1 μl = 0.4u | 37° C., 45° C. |
| | Mn2+ | | 55° C., 65° C. |
| E. coli ligase | X10 E. coli ligase buffer | 0.4 μl = 4u | 16° C., 37° C. |
| Taq DNA | X10 Taq ligase buffer | 0.4 μl = 16u | 37° C., 45° C. |
| ligase | | | 55° C., 65° C. |
| Ampligase | X10 ampligase buffer | 1 μl | 37° C., 55° C. |

Indexers were used in combinations from the following list which also indicates their target fragments:

| | |
|---|---|
| Hex-TTTTAGTCTACT | 140 base Hinfl fragment |
| Hex-TTTTAGTCATTT | 140 base Hinfl fragment (opposite |
| | end to above) |
| Hex-TTTTAGTCGAAA | 151 base Hinfl fragment |
| Hex-TTTTAGTCTTCT | 207 base Hinfl fragment |
| Hex-TTTTAGTCAAGT | 720 base Hinfl fragment |
| Hex-TTTTAGTCGCCA | 720 base Hinfl fragment (opposite |
| | end to above) |

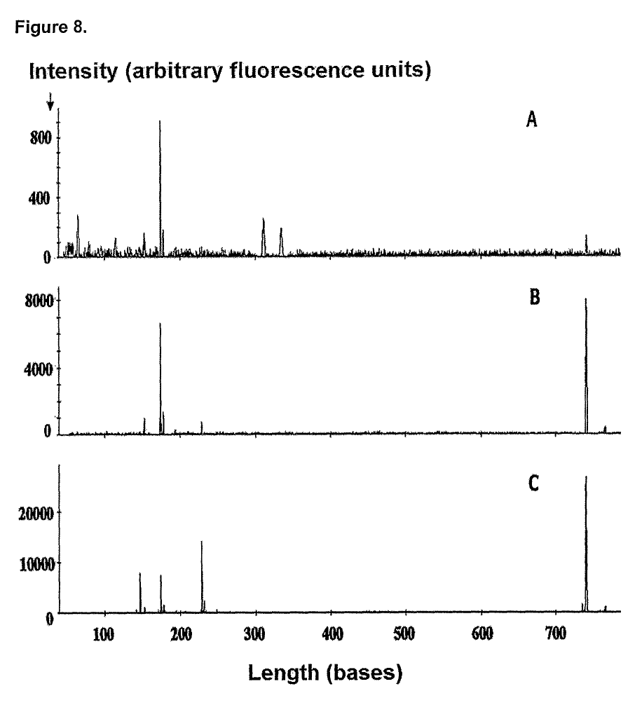

Results are shown in part in FIG. 8. The weakest results were obtained with Pfu ligase under the standard conditions where only the 151 base fragment and one end of the 720 base fragment were strongly detected. The upper electropherogram (FIG. 8A) shows results with Pfu DNA ligase at 37° C. with magnesium which shows a slight improvement. In the middle electropherogram (FIG. 8B), the best results for this enzyme are shown where manganese had been used at 37° C. The 720 base fragment was now detected with the MGT ended indexer but the 140 and 207 base fragments still have weak signals. Taq ligase used at 45° C. (FIG. 8C) gives good signals with all of the fragments even with magnesium. The slight differences are in part related to the lower recovery on purification of smaller fragments. We have standardised on Taq ligase at 45° C. and it is a matter for the user to determine their preferred conditions.

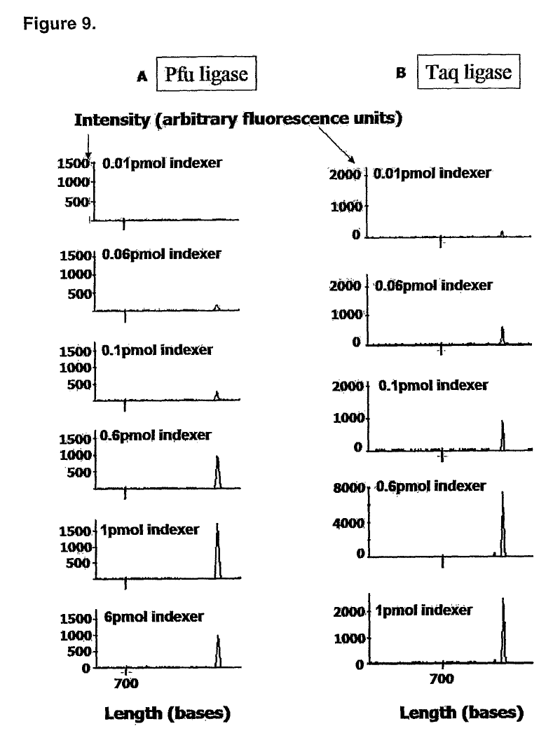

Assays of this type can be used to determine other optimal parameters for example the amounts of ligase and amounts of indexer required. The latter is of particular importance because our process uses mixed pools of indexers. Having more indexers per pool allows more different types of ends to be accessed. However, this increases the total mass of DNA in the reactions and as indexers become longer, ultimately numbers can become prohibitive. FIG. 9 shows the effects on yield of labelled product when the concentrations of indexer were varied in the reactions described above. There were 0.009 pmoles of the 720 base target fragment per reaction. The optimum amount of indexer the indexer ending AAGT used with Pfu DNA ligase was 1.0 pmoles but yields were still markedly less than for Taq DNA ligase which optimally required 0.6 pmoles of indexer. There was approximately a 67 molar excess of indexer over target, substantially more than absolutely required. These concentrations of indexer are used to drive the reaction and can support the use of more target.

### EXAMPLE 5

### Indexing at a Blunt End

The process described in example 2 demonstrates that indexing molecules can be placed with high sequence specificity on the ends of target molecules. We show here that this is also possible when the original fragments have blunt ends. Blunt ends are expected on the ends of fragments where actual sequence information is obtained for sequence assembly. In this example DNA from the bacteriophage lambda (NEB) was cut to completion with HincII for use as a target. HincI has the advantage that it naturally leaves blunt ends but the recognition sequence is degenerate so that a range of possible ends are found in the population as a whole. The purified fragments were ligated using T4 DNA ligase (NEB) to an 80 pmolar excess of blunt ended adapters. Four types of adapter were compared

| | |
|---|---|
| FaNBsXbU8b | 5′ FAM AGATCCTGAGAATCTCAGATCTAGAGTC |
| With | |
| NBsXbL8b | 3′ TCTAGGACTCTTAGAGTCTAGATCTCAG |
| HeNBsXbU5b | 5′ HEX TCCTGAGAATCTCAGATCTAGAGTC |
| With | |
| NBsXbL5b | 3′ TAGGACTCTTAGAGTCTAGATCTCAG |
| HeNBsXbU3b | 5′ HEX CTGAGAATCTCAGATCTAGAGTC |
| With | |
| NBsXbL3b | 3′ GGACTCTTAGAGTCTAGATCTCAG |
| HeNBsXbU1b | 5′ HEX GAGAATCTCAGATCTAGAGTC |
| With | |
| NBsXbL1b | 3′ ACTCTTAGAGTCTAGATCTCAG |

In each case the 5′ end of the upper strand of the adapter was labelled and blocked by the fluorescent dye named. The lower strand had a 5′ phosphate added by T4 polynucleotide kinase (NEB). Kinasing, annealing, ligation and removal of unligated adapters were all as described in Example 2. The adapters have sites for the nicking endonuclease N. BstNBI and the endonuclease XbaI so that an 8 base 3′ overhang can be produced by the respective action of these enzymes. 10 units per ug of N.BstNBI (NEB) were used overnight at 55° C. in 50 ul buffer 3 (NEB) per ug and the reaction adjusted to 100 ul of buffer 2 (NEB) per ug and 10 units/ug of XbaI (NEB) and incubation continued for a further 3 hours at 37° C. Controls equivalent to 0.5 ug of material sampled after each stage of the process were examined by electrophoresis through an agarose gel and visualised using a Typhoon Fluorescent Scanner (GE healthcare) pre staining and post staining with SYBR Gold. Significantly, residual (unpurified) adapters were present for the sample corresponding to FaNBsXbU8b/NBsXbL8b whereas these had been largely removed in the other cases. Unpurified material interferes with the digests and the indexing ligations that follow. The adapter FaNBsXbU8b/NBsXbL8b that was not removed efficiently by the procedure is the longest and this reflects the importance of having adapters whose length (design) is matched to the method of purification. The dyes used do not make a significant difference to the purification.

Final purified material in each of the remaining samples were ligated to each of the possible indexers:

| | |
|---|---|
| FANBs11aaca | 5′ FAM TTTTAGTCAATA |
| FANBs11aacc | 5′ FAM TTTTAGTCAATC |
| FANBs11aacg | 5′ FAM TTTTAGTCAATG |
| FANBs11aact | 5′ FAM TTTTAGTCAATT |
| FANBs11gaca | 5′ FAM TTTTAGTCGATA |
| FANBs11gaca | 5′ FAM TTTTAGTCGATC |
| FANBs11gaca | 5′ FAM TTTTAGTCGATG |
| FANBs11gaca | 5′ FAM TTTTAGTCGATT |

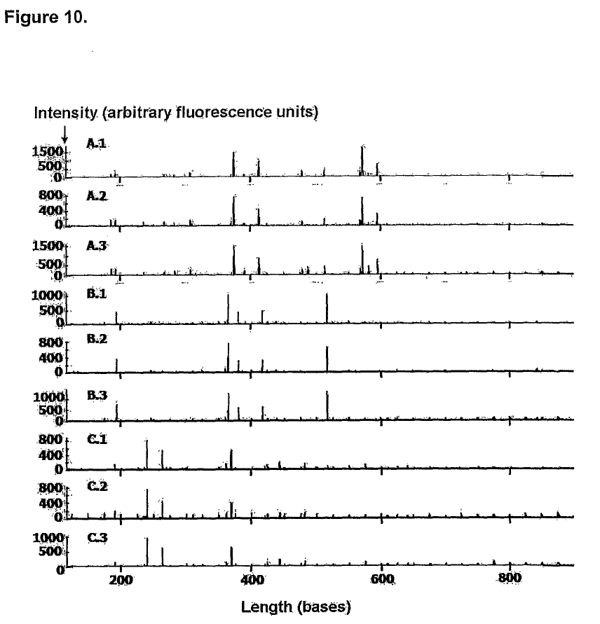

The successful samples and the indexers were used separately making 24 reactions in all. Reactions were according to the best conditions described in example 4. Low molecular weight material was removed from the ligations by ultrafiltration (Microcon 50, Amicon) and the samples analysed by capillary electrophoresis using a MegaBACE System (GE Healthcare). Representative electropherograms are shown in FIG. 10. The fragments observed are as expected from the known sequence of bacteriophage lambda and the enzymes and indexers used. Each electropherogram in a triplet was produced using a different starting adapter. Note the high degree of reproducibility in the totally independent reactions.

### EXAMPLE 6

### Indexing of Human Genomic DNA

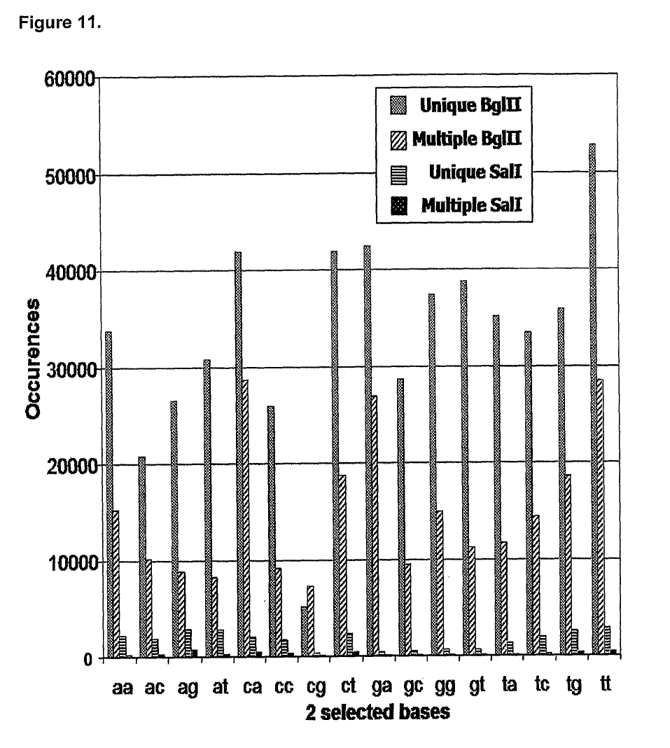

The process provides a general means of sequencing capable of reading the entire genome of a higher eukaryote like the human. This requires a suitable frequency of the fixed ends shown in FIG. 7 to achieve coverage of the genome. We have performed a search of the human genome sequence NCBI build 35.1 using the algorithm fuzznuc—see above to check that unique sites do exist at a suitable frequency to support our process. The results are summarised in FIG. 11, where the results for enzymes that would be expected to cut more (BglII) and less (SalI) frequently are compared. There is an abundance of unique sites at our suggested level of selection (18 bases total), and the overall frequencies are much as expected from known dinucleotide frequencies; i.e. CpG containing sites are under-represented. It is therefore a matter for one skilled in the art to adopt a strategy suitable for their particular needs and given the wide availability of restriction enzymes and the universal nature of our approach there are no limitations.

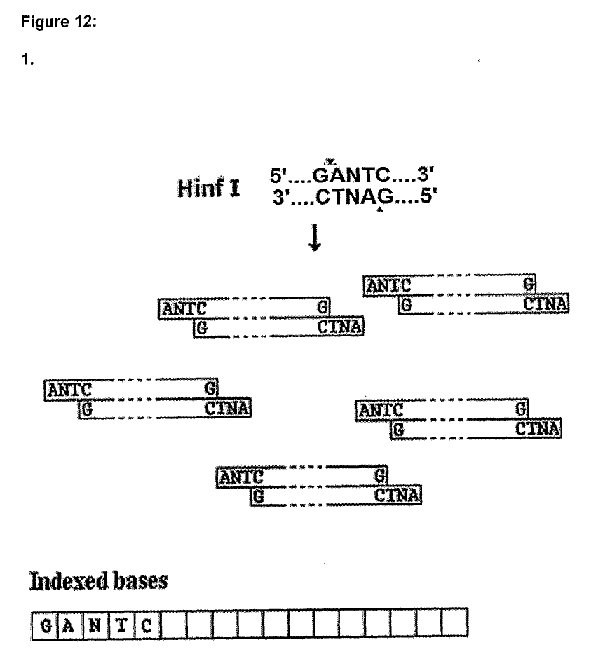

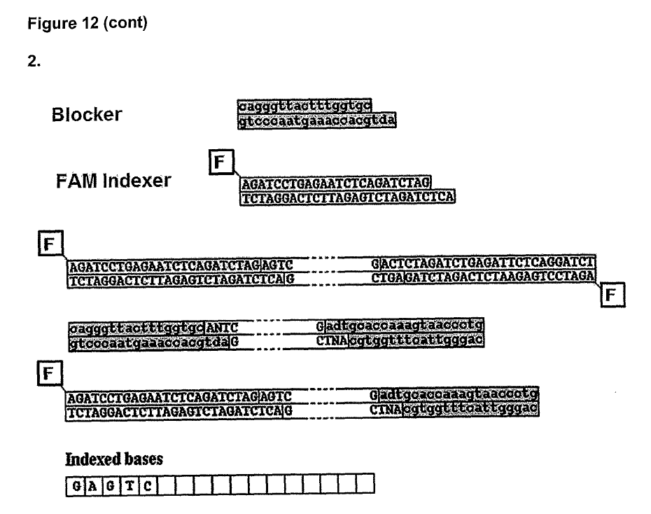

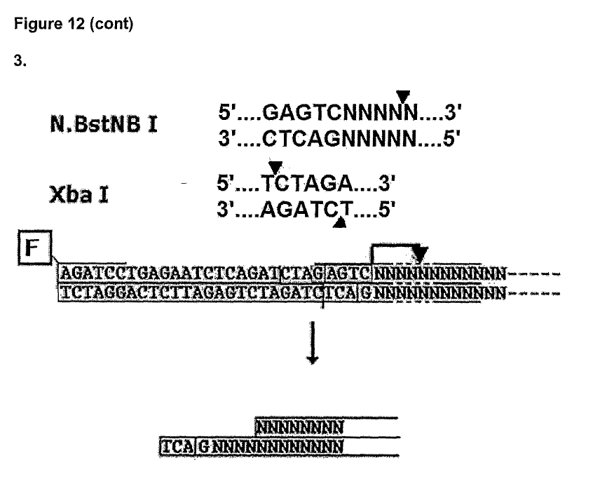

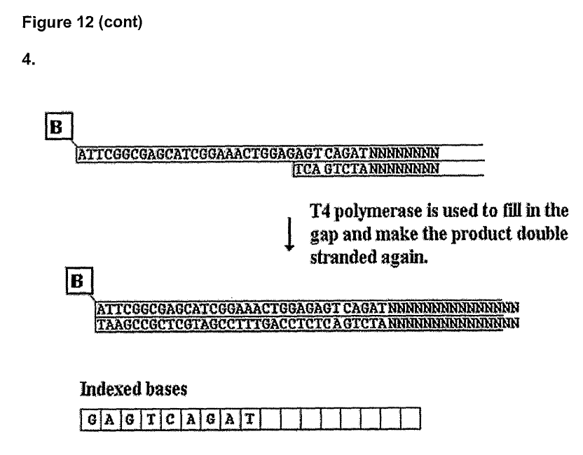

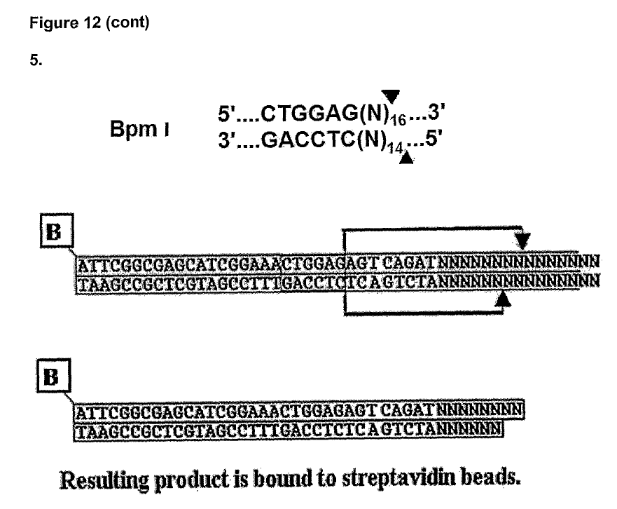

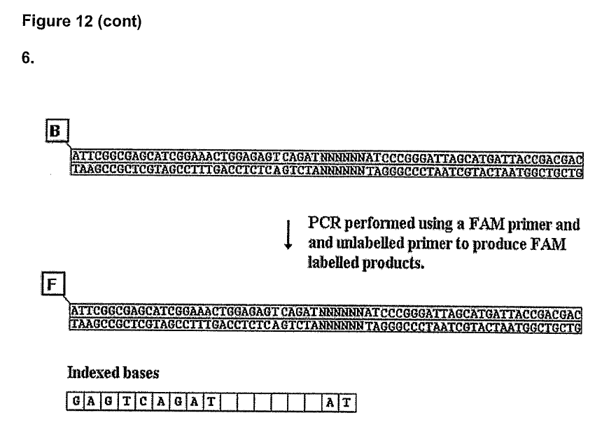

Indexing does indeed sample the target sites as expected as shown in this example. The approach is shown in FIG. 12, where a wide range of short sequences were isolated from sites in the human genome that were not pre-selected. A single indexing adaptor was ligated to a complete Hinf1 digest, selecting ¼ of the available 3-base overhangs. This indexer placed nicking and cleavage sites as described above to allow 8-base overhangs at each end to be created, with 4 known and 4 unknown bases. Ligation of single indexers selected 1/256 fragments at each end. Sequences at the 2 ends of each molecule were then analysed separately, following cleavage (step 5) by a Type IIS endonuclease directed from the indexer. T4 DNA ligase was used in step 6 to ligate a single indexing adaptor to the results of this cleavage, which left an undefined 2-base overhang. Thus we selected 1/16 fragments at step 6: a total enrichment of 1/16,384. We expected 7⁴ different fragments with the constant features indicated in FIG. 12, step 6.

### First Adapter Ligation:

8 μg of HinfI digested human genomic DNA.
240 pmol adapter set 1 (TCA) per μg of DNA [1920 pmol]
720 pmol adapter set 2 (TDA blocker) per μg of DNA [5760 pmol].
1×T4 DNA ligase buffer (NEB).
24 μl T4 DNA ligase (400 units/μl, NEB).
H₂O to volume of 4880 μl.

Adapter set 1 (TCA)
5′ FAM AGATCCTGAGAATCTCAGATCTAG
with
3′ TCTAGGACTCTTAGAGTCTAGATCTCA
Adapter set 2 (TDA)
5′ CAGGGGTTACTTTGGTGC
with
3′ GTCCCCAATGAAACCACGTDA

### D=A, G, T mix

Incubation was performed at 16° C. for 16 hours.

Purification was as described in example 2 and elution in 60 μl of EB yielding ˜7.5 μg adaptored DNA.

NBstNBI was used for nicking:

### 7.5 μg DNA.

1×NBstNBI buffer (NEB).
10 units/μg DNA NBstNBI (75 U, NEB).
H₂O to volume of 200 μl.

Incubation was at 55° C. for 16 hours.

XbaI was used for the double stranded cleavage:

### 7 μg DNA.

1× buffer 2 (NEB).
1 mg/ml (68.8 μg BSA, NEB).
17 units/μg DNA XbaI (120 U NEB).
H₂O to volume of 688 μl.

Incubation was at 37° C. for 90 mins.

The short fragments of adapter plus end sequence were removed as described in example 2 by heating to 75° C. and purifying to yield ˜6.5 μg DNA.

Indexing was performed with a biotinylated indexer:

0.14-0.19 μg DNA (NBstNBI/XbaI digest above)
25 pmols Biotin indexer oligonucleotide
1×Pfu DNA ligase buffer (Stratagene)
4 U Pfu DNA ligase (Stratagene).
H₂O to volume of 100 μl.

### Biotin Indexer Oligo:

| | |
|---|---|
| 5′ Biotin | ATTCGGCGAGCATCGGAAACTGGAGAGTCAGAT |

Incubation was performed with continuous cycles of: 72° C. 30 sec, 45° C. 3 mins, 50° C. 3 mins, 55° C. 5 mins for 16 hours

The single stranded region of the ligated indexer was then filled by T4 DNA polymerase:

˜14 ng-19 ng biotin adapted DNA.
1× buffer 3 (NEB).
16 nmols dNTPs
H₂O to volume of 80 μl.

Incubation was performed at 12° C. 30 mins followed by 75° C. 15 mins. and −20° C. for 90 mins.

Sample was purified as standard and eluted in 30 μl H₂O.

Short fragments were released from the indexed fragments by the TypeIIS endonuclease BpmI:

˜14 ng-19 ng T4-filled DNA.
1× buffer 3 (NEB).

### 20 μg BSA.

### 5 U BpmI (NEB).

H₂O to volume of 200 μl.

Incubation was at 37° C. for 3 hrs followed by heat inactivation at 65° C. for 20 mins.

The biotinylated material was purified by binding to streptavidin coated paramagnetic beads, a PCR able adapter added to the non indexed end and amplified by PCR:

The entire sample was bound to 100 μg of dyna beads (DynaI) and washed as recommended by the supplier. The PCR adapter (AT) was added after the washes:

1 pmol PCR adapter
1×T4 DNA ligase buffer (NEB)
200 U T4 DNA ligase (NEB)
H₂O to volume of 50 μl.

PCR adapter (AT)
5′ GTCGTCGGTAATCATGCTAATCCCGGGAT
with
3′ CAGCAGCCATTAGTACGATTAGGGCCCTA

The reaction was performed at ambient for >=1 hr.

The beads were added directly to the PCR:

The beads were washed with 2×BB (DynaI as supplied), 0.1 M NaOH (5 mins), 0.1 M NaOH, 1×BB then 50 μl of 1×PCR buffer was added to the beads. The beads were then split into two aliquots of 25 μl and 2 PCR reactions per sample were set up.

˜2.5 ng-5 ng DNA.
1×PCR buffer (Hot start Qiagen).

### 0.05 μM Mg2⁺

5 nmols dNTPs
25 pmols primer F
25 pmols primer R
2.5 U Hot Start Taq polymerase (Qiagen).

| | |
|---|---|
| primer F 576P | 5′ ATTCGGCGAGCATCGGA |
| primer R 228p | 5′ GTCGTCGGTAATCATGC |

PCR products were purified as standard and cloned using TOPO cloning kit as recommended (InVitrogen). Inserts were amplified using the M13 reverse and −21 forward primers, prepared for sequencing by the ExoSAP-IT system (GE Healthcare) and sequenced using the MegaBACE capillary electrophoresis system (GE Healthcare). Sequences obtained were compared using the algorithm Blast, to the NCBI build 35.1 human sequence. The results are summarised in table 3. Unique means a unique match to the human genome whilst Multiple means a match to a repetitive sequence.

The 85 sequences all show the predicted constant features and the data indicate that both unique and repeat sequences have been sampled. There are 18 instances where there is no perfect match. In all cases, these are mismatched at one or both bases of the 3′-terminal AT, and/or at the 5′-terminal G. The latter is part of the HinfI site, and therefore must be present in our starting DNA. The 6 definite cases in which mismatches occurred at the 5′-G are likely polymorphic sites. In 4 further cases, the top hits were a mixture of 5′-G and 3′-T mismatches it is not possible to distinguish the experimental results from the genomic data. There are 14 examples of either 1 or both bases mismatched at the 3′-terminal AT. These bases were introduced at step 6 by T4 DNA ligase. We expect ˜85% fidelity from this enzyme in the presence of a single adaptor (Sibson and Gibbs), and the observed 16% mismatch at these sites is consistent with this level. No mismatches were observed in the sequences corresponding to the region selected by indexing at the site with the 8 base 3′ overhang. Thus the iterative indexing is effective and the fidelity of the new indexing procedure is good with human genomic DNA. Uniquely, we have a useful method for global exploration of sequence representation levels, and we have demonstrated the effectiveness of iterative indexing and the fidelity of our long-overhang indexing approach. This experiment shows the use of multiple cycles of indexing, yields sequences from the genome that are consistent with high fidelity indexing and unique sequences are obtained at a useful frequency. Note that the reduced fidelity obtained with T4 catalysed ligations are irrelevant for the full process. In all cases the strand which serves as the template for high fidelity indexing originates from the target fragment not the added adapters. Therefore ends receiving an inappropriate adapter should they proceed to a next round of indexing will have the original correct sequence present after the adapter has been removed by nicking and the double stranded endonuclease.

TABLE 3
Test of Allelic Imbalance Protocol: Categories of Best Matches

| | | | | | | Mismatch | |
|---|---|---|---|---|---|---|---|
| | | Mismatch | Mismatch | Mismatch | Mismatch | 5′G + | |
| | Match | 5′G | 3′T | 5′G or | 3′TA | 3′T | |
| | (17/17) | (16/16) | (16/16) | 3′T*(16/16) | (15/15) | (15/15) | Totals |
| Unique | 41 | 2 | 1 | 0 | 0 | 2 | 46 |
| Multiple | 26 | 2 | 6 | 4 | 1 | 0 | 39 |
| Totals | 67 | 4 | 7 | 4 | 1 | 2 | 85 |

*hits included bases 1-16 and 2-17 matches.

### EXAMPLE 7

### Circularisation of a Target Fragment Having Indexers at Both Ends

The key circularisation reaction required for bringing the indexers together post indexing is demonstrated here. f X174 RF DNA was used here for making the indexers to demonstrate the practicability of the process with long indexers. The f X174 DNA prepared in example 4 was used for a target.

### Preparation of Indexers

For the indexer f X174 was prepared as follows:

### 30 μl f X174 RF1 DNA (NEB)

60 μl PstI (20 units/μl NEB)
120 μl ×10 buffer 3 (NEB)
990 μl water

Incubation was at 37° C. for 3 hrs, 60 μl of BssHII (4 units/ul NEB) were added and incubation continued at 50° C. for 3 more hours. Standard purification was used as above.

Each indexing molecule has an indexing end ligated onto the PstI cleavage produced end and an end for recircularisation on the end produced by BssHII cleavage. It is important that the latter lacks a 5′ phosphate or indexer joining can occur prematurely. The indexing end was therefore produced using the following adapters:

| | |
|---|---|
| PxI1Hf/U: | 5′ GATGTGGGTTTGCA |

Annealed to pxI1Hf/NNNN or pxI2Hf/NNNN where NNNN are the 4 nucleotides complimentary to the specific nucleotides revealed by the NBstNBI/XbaI digest of target DNA in example 4 above.

PxI1Hf/NNNN:
5′AACCCACATCTACAGACCCTGAAGAAACAGTCNNNN
Or
PxI2Hf/NNNN:
5′AACCCACATCTACAGACCAAGCTGAAACAGTCNNNN

The significant difference between PxI1 and PxI2 is that the former contains an AcuI site from which a second round of indexing can be initiated if required after recircularisation.

Note that PxI1Hf/U does not anneal to the full length of PxI1Hf/NNNN or PxI2Hf/NNNN even after the latter have annealed at their opposite end to the indexed target. Thus a single stranded gap remains in each of the indexers, adjacent to the target sequences. Second round indexing is enabled by digestion from the Acu I site following filling of the gap, either by ligation of the corresponding oligonucleotide in particular for or by the action of a DNA polymerase. The oligonucleotide(s) first have a phosphate added to their 5′ ends as above and are added in a 2 to 10 molar excess for ligation as above. Filling by a polymerase is conveniently achieved by T4 DNA polymerase as described for preliminary preparation of the target above.

The recircularisation ends were produced using the following adapters:

| | |
|---|---|
| PxCIRC/U: | 5′ CGCGAGGGAGATCACTTC |

Annealed to pxCIRC/GACT or pxCIRC/AGTC, that is two non-palindromic complimentary overhangs which should not ligate unless they are phosphorylated.

| | |
|---|---|
| pxCIRC/GACT: | 5′GACTGAAGTGATCTCCCT |
| pxCIRC/AGTC: | 5′AGTCGAAGTGATCTCCCT |

For joining the adapters to the cleaved f X17 DNA the adapters pxI1Hf/NNNN, pxI2Hf/NNNN and pxCIRC/U were separately kinased as follows:

### 75 μl ATP 10 mM

75 μl ×10 PNK buffer (NEB)
60 μl oligo 50 pmol/μl
540 μl water
15 μl polynucleotide kinase (10 units/ul NEB)

Incubation was performed at 37° C. for 3 hours and the polynucleotide kinase was inactivated by heat at 95° C. for 10 mins. An equal volume of the appropriate complementary oligonucleotide at 4 pmol/ul was added and the two oligonucleotides annealed at 60° C. for 10 minutes.

A test ligation was performed to confirm that the appropriate ends were present:

One end, i.e. either pxIHf (ds), or pxI2 (ds), or CIRC/GACT (ds), or CIRC/AGTC (ds); was ligated to the f X174/PstI/BssHII digested DNA. (ds) denotes double stranded.

2 μl f X174/PstI/BssHII digested DNA
11 μl one end (ds) oligo (2 pmol/μl)
1.4 μl ×10 T4 DNA ligase buffer (NEB)
0.2 μl T4 DNA ligase (400 units/ul NEB)

Incubation was performed at 16° C. for 16 hours and the products analysed by agarose gel electrophoresis. If the digest had worked well the f X174 molecules ligate to the (ds) oligo at one end and be “capped” by it but the other end of the molecule can dimerise with a second f X174 molecule. Usable material was obtained if more than 80% formed a dimer band at ca.10.6 kilobases.

Indexers for use were prepared by a bulk ligation:

54 μl f X174/PstII/BssHII digested DNA
69 μl pxI1Hf/NNNN (ds) 2 pmol/μl
69 μl CIRC/GACT (ds) 2 pmol/μl
18 μl ×10 T4 DNA ligase buffer (NEB)
40 μl water
2.5 μl T4 DNA ligase (400 units/ul NEB)

Indexer 1 was usually used with CIRC/GACT and Indexer 2 was used with CIRC/AGTC, although as long as every ligation contained one indexer end and one circularisation end the exact pairing does not matter. However when pairs of indexers are used in indexing reactions it is important that one of the pair has CIRC/GACT and the other CIRC/AGTC. Otherwise circularisation cannot occur.

Incubation was perfomed at 16° C. overnight.

Purification was as standard above followed by size exclusion chromatography (Chromospin 1000+TE column, Clontech) used as recommended. The latter was required to remove excess oligonucleotides which otherwise would compete unfavourably with the indexer in the reactions that follow.

To confirm that the new indexers were functional in joining at the ends for circularisation as required phosphates were added to their free 5′ ends and they were allowed to ligate.

300 ng indexer I1 or I2
2 μl ×10 T4 DNA ligase buffer (NEB)

### 2 μl ATP 10 mM

water to a final volume of 20 μl
0.4 μl T4 Polynucleotide kinase (10 units/ul NEB)

Incubation was performed at 37° C. for 3 hours.

Ligations were then performed as follows:

Reaction A: f X174 indexer I1, Polynucleotide kinase treated, self-ligated.
Reaction B: f X174 indexer I2, Polynucleotide kinase treated, self-ligated.
Reaction C: f X174 indexer I1, Polynucleotide kinase treated+f X174 indexer I2, Polynucleotide kinase treated+ligase
Reaction D: f X174 indexer I1+f X174 indexer I2+ligase (no Polynucleotide kinase treatment).

### Reactions A, B and C:

10 μl Polynucleotide kinase treated f X174 indexer DNA (5 μl I1+5 μl I2)
0.2 μl T4 DNA ligase

### Reaction D

100 ng f X174 indexer I1
100 ng f X174 indexer I2
water to final volume of 10 μl
1 μl X10 T4 DNA ligase buffer (NEB)
0.2 μl T4 DNA ligase (400 units/ul NEB)

All reactions were incubated at 16° C. overnight and analysed by agarose gel electrophoresis.

The majority of the material shifts on ligation to the 10.6 Kb form from 5 Kb showing that the required ends are functional.

To confirm that the indexing ends were also present they were ligated to a labelled test oligonucleotide as follows:

5 μl f X174 indexer I1, polynucleotide kinase treated or f X174 indexer I2, polynucleotide kinase treated
0.5 μl pxI1HfQC (4 pmol/μl)
2 μl ×10 Taq DNA ligase buffer (NEB)
12.5 μl water
0.8 μl Taq DNA ligase (40 units/ul NEB)

| | |
|---|---|
| pxI1HfQC: | 5′(HEX) TTCAGGGTCTGTA |

Incubation was performed at 45° for 16 hours. The products were analysed by agarose gel electrophoresis and visualised unstained using a Typhoon Fluorescent scanner (GE Healthcare and then after staining with Syber Gold. It is possible to detect the f X174 indexers before staining because the labelled oligonucleotide is able to ligate to the ends for indexing where they are present.

### Indexing and Circularisation

In order to achieve the concentrations of material to be circularised f X174 derived indexers and target prepared as above were evaporated to dryness in an (Gyrovap, Howe), avoiding over drying.

1 μg f X174 indexer1/AAGT
1 μg f X174 indexer2/GCCA
1 μg target f X174/HinfI DNA

The DNA was redissolved in water 18 μl and 2 μl ×10 Taq DNA ligase buffer (NEB). 1 μl Taq DNA ligase (40 units/ul NEB) were added and ligation allowed to proceed for 16 hrs at 45° C. The reaction was sampled (3.5 ul) for analysis by agarose gel electrophoresis and the remainder purified as standard. Purified material was eluted in 30 ul of EB (supplied). Phosphates were added to the ends for circularisation by T4 polynucleotide kinase and the indexed molecules were circularised by ligase as follows:

| | |
|---|---|
| 30 μl | purified DNA in EB |
| 4 μl | X10 polynucleotide kinase buffer (NEB) |
| 4 μl | ATP 10 mM |
| 1 μl | water |
| 1 μl | T4 polynucleotide kinase (10 units/ul NEB) |

Incubation was performed at 37° C. for 3 hours.

1 μl of T4 DNA ligase (400 units/ul NEB) were added and the reaction continued at 16° C. for 16 hrs before purifying as standard and elution in 30 ul of EB supplied.

A sample (3 μl) was removed for analysis by agarose gel electrophoresis. The remaining material was cleaved by restriction endonucleases to produce characteristic restriction fragments that would indicative that circularisation had occurred and the indexers had joined as a result.

### First Reaction:

13.5 μl purified DNA from above
2 μl ×10 buffer 4 (NEB)
2.5 μl water
2 μl Mfel (10 units/μl, NEB)
13.5 μl purified DNA from above
2 μl ×10 buffer 2 (NEB)
2.5 μl water
2 μl SacII (20 units/μl)

Restriction digests were performed for 2.5 hrs at 37° C. and then analysed with the samples above by agarose gel electrophoresis.

The anticipated fragments are shown in table 4 below in their linear order.

| | | SacII digest | MfeI digest |
|---|---|---|---|
| Composition | Form | fragments (bp) | fragments (bp) |
| Indexer 1, target | circular | 6450, 4972 | 8604, 2818 |
| Indexer 2 | | | |
| Indexer 1, target | linear | 6450, 2486 | 8604, 1409 |
| Indexer 2 | | | |
| Indexer 1 or 2 | linear | 2486, 2865 | 1409, 3942 |
| Indexer 1 or 2 | linear | 3585, 2486 | 4662, 1409 |
| target | | | |
| 2 Indexers | linear | 4972, 2865 | 2818, 3942 |
| Indexer, indexer | linear | 3585, 4972, 2865 | 4662, 2818, 3942 |
| target | | | |

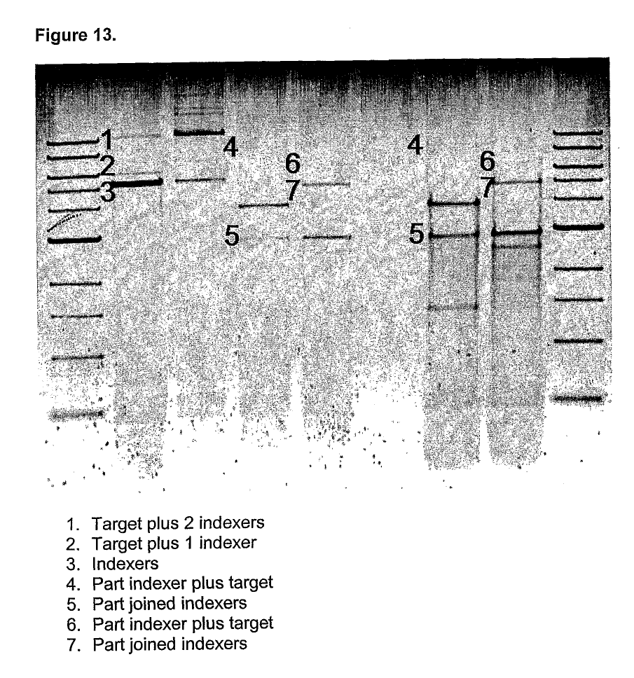

The bands signifying circularisation are sized 8604 and 2816 for the Mfel digest and 6450 and 4972 for the SacII digest and are seen with the other bands in FIG. 13 as expected.

There is a single stranded gap in each of the indexers, adjacent to the target sequences opposite the sequences 5′ tacagaccctgaagaaac or 5′ tacagaccaagaagaaac for the first and second indexers respectively. In order to enable second round indexing, these can be filled, either by ligation of the corresponding oligonucleotides in particular for the first one to complete the Acu I site or by the action of a DNA polymerase. The oligonucleotide(s) first have a phosphate added to their 5′ ends as above and are added in a 2 to 10 molar excess for ligation as above. Filling by a polymerase is conveniently achieved by T4 DNA polymerase as described for preliminary preparation of the target above.

### EXAMPLE 8

### Indexer Structure and Labelling

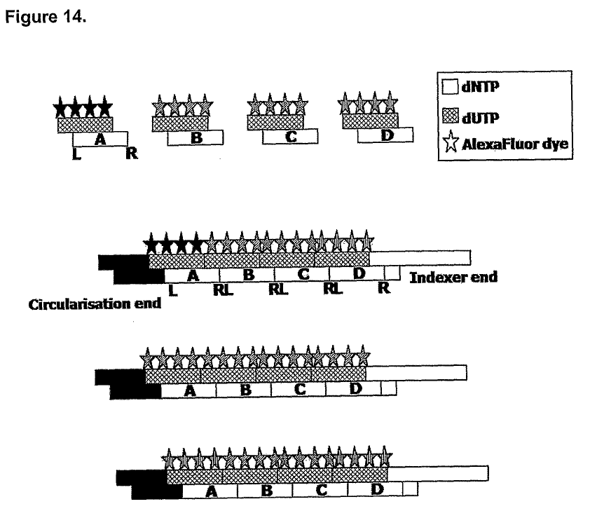

We have developed two different strategies for indexer labelling for MIDAS. The first was based on long indexers, each with 4×˜1 kb labelled sections. Indexers of this length have been used routinely (Sibson and Gibbs, 2002, Nucleic Acids Res. 2001; 29: e95 incorporated herein by reference for all purposes). The important feature of indexers for ligation to target is not their overall length but the length of their cohesive end available for indexing. The encoding of sequence for the 4 discriminating bases is simple: each 1 kb section represents a different discriminating base, in the same spatial order, and is labelled in 1 of 4 colours. Published information shows that, 1 kb sections labelled with at least 5 fluorophores can be discriminated spatially by CCD imaging (Femino A et al, Science. 1998; 280:585-90 incorporated herein by reference for all purposes). The first strategy is outlined in FIG. 14. PCR using the primers from table 5 below was used to isolate ˜1 kb fragments from the kanamycin resistance transposon Tn903. Extra sequences on their ends allowed them to be cloned with particular orientation by standard cloning techniques into the phagemid pBluescript SK−. The important features of the constructs are BglI and Eco019I sites that flank the inserts and allow the fragments to be excised for concatermerisation in a predetermined order, preserving any preexisting labelling relationships as shown in FIG. 14. A second feature is that the nucleotide t was avoided in the regions that flank the insert. This allowed one strand of the phagemid to be produced as single stranded DNA to be produced by standard techniques and then labelled by incorporation of allyl dUTP (InVitrogen) without risk of the label interfering in the regions at the ends that were to participate in further manipulations. The allyl dUTP allowed incorporation of any convenient dye for labelling prior to concatamerisation. Oligonucleotide indexing adapters and adapters to allow circularization as above are included in the concatamerisation. They have 2 functions. The first is to act as the actual indexing sites or sites for circularization as appropriate. The second is to control concatamerisation to the required 4 kb products. This approach was aimed at using directly labelled indexers but of course one skilled in the art will appreciate that it can easily be modified for indirect labelling i.e. as a hybridization probe by substituting the indexing adapter by a suitable probe sequence.

A second strategy was aimed at secondary detection but it will be appreciated when compared to the first approach above that it can be suitably modified for direct detection. We call the approach Catherine Wheel and it was originally for detecting the probe target on the branched indexers. It provides high specificity, high label density, high labelling flexibility for information encoding, with a wide variety of label combinations. Single stranded DNA is used as a scaffold, to link many different separately-labelled probe elements (see FIG. 15). The elements comprise double stranded fragments flanked by PCR primer sequences and corresponding to the single stranded scaffold. The PCR primer sequences either comprise a probe complementary to one of the indexer branch arms on one of the indexer types or flank the 5′ end of such a probe. Probe elements are made simply by PCR, and the Catherine Wheel is formed by annealing these PCR products to single-stranded M13 DNA. Labelling can either be through the use of labelled primers or through direct incorporation during synthesis or both.

We have produced labelled segments both by using universal labelled primers and by incorporating internal labels during PCR.

The oligonucleotides are listed below.

Kp17SU
ACGAGTACATCGAACTACGGCTAG
Kp27SU
GGAGTTCTGCATCACTTGACGCTA
EV17SU
TGTCGGTGCGTCATTTCTAAGGAG
EV21SU
GGATCCGAATTCACTCGGGCCAAG
Kp17Lms
TACTAGCCGTAGTTCGATGTACTCGT
Kp27Lms
TATAGCGTCAAGTGATGCAGAACTCC
EV17Lms
TACTCCTTAGAAATGACGCACCGACA
EV21Lms
TACTTGGCCCGAGTGAATTCGGATCC
Kp17SU228EV
5′GTCGTCGGTAATCATGCGATATCACGAGTACATCGAACTACGGCTAG
Kp27SU228EV
5′GTCGTCGGTAATCATGCGATATCGGAGTTCTGCATCACTTGACGCTA
EV17SU228EV
5′ GTCGTCGGTAATCATGCGATATCTGTCGGTGCGTCATTTCTAAGGAG
EV21SU228Ev
5′ GTCGTCGGTAATCATGCGATATCGGATCCGAATTCACTCGGGCCAAG
Kp17Lms228EV
5′ TACTAGCCGTAGTTCGATGTACTCGTGATATCGCATGATTACCGACGAC
Kp27Lms228Ev
5′ TATAGCGTCAAGTGATGCAGAACTCCGATATCGCATGATTACCGACGAC
EV17Lms228EV
5′ TACTCCTTAGAAATGACGCACCGACAGATATCGCATGATTACCGACGAC
EV21Lms228EV
5′ TACTTGGCCCGAGTGAATTCGGATCCGATATCGCATGATTACCGACGAC
228P_Ax350
5′ Alexa ® 350 GTCGTCGGTAATCATGC
228P_Ax488
5′ Alexa ® 488 GTCGTCGGTAATCATGC
228P_Ax546
5′ Alexa ® 546 GTCGTCGGTAATCATGC
228P_Ax568
5′ Alexa ® 568 GTCGTCGGTAATCATGC
228P_Ax680
5′ Alexa ® 680 GTCGTCGGTAATCATGC

M13mp18 conveniently has 63 sites for the restriction endonuclease MseI. Each of these can be adaptored and amplified by PCR. Label can be incorporated during synthesis either through a labelled primer or using labelled oligonucleotides or both. The lower strands of the adapters are denoted Lms in the names above and they are complementary to their matching named oligonucleotide denoted SU in the names above. The final 5 oligonucleotides named 228P are the primers for primer labelling with their corresponding dye. The primers are universal and can be used with any of the adapter pairs named 228. The 3′ ends of the upper adapters in each case correspond to probe sequences for the branched oligonucleotides below. In the case of the shorter adapter pairs the adapters also correspond directly to the probe sequences and the upper strand is also the primer strand. Chromatide nucleotides (In Vitrogen) were used for internal labelling as recommended by the supplier.

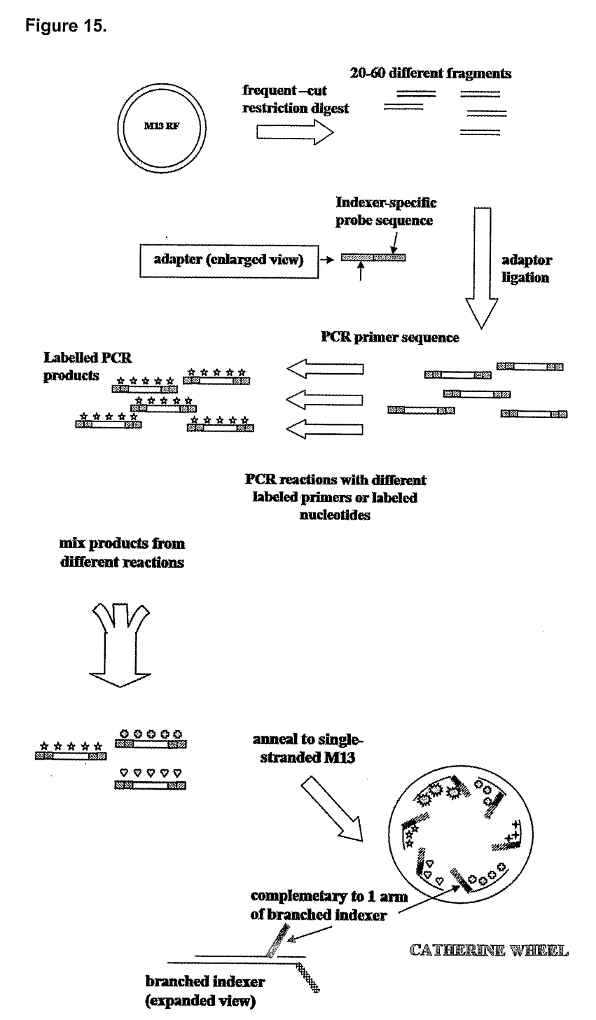

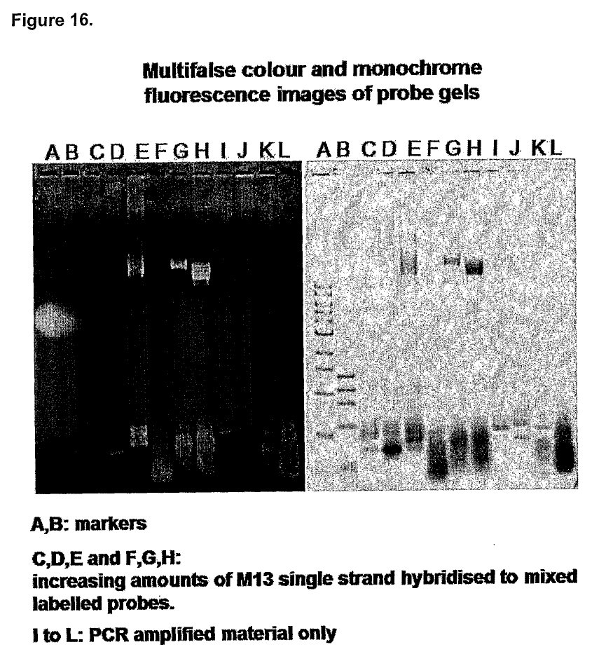

Probes with internal labels proved to be more fluorescent but both types are usable. Examples are shown in FIG. 16. The amounts of single-stranded M13 DNA used for hybridisation was also varied here and this is reflected in the relative amounts of probes produced. The ethidium stained gel on the right of FIG. 16 shows that significant quantities of probe can be produced.

It will be appreciated that these approaches represent extremely flexible and robust systems allowing for precise control of the labels, their ratios and the probe sequences. Our preferred system advantageously combines elements of both. Here, the labelling of the required segments is performed to produce a single probe recognition site per molecule, an enormous advantage in terms of avoiding cross reactivity between probes having the same target recognition regions as can occur with the Catherine Wheel approach. f X174 single stranded DNA is used as the scaffold because it is smaller and the larger size of M13 is not necessary. Separate ˜1 kb segments of f X174 are cloned by standard techniques into bacteriophage M13 SK−. Synthesis from a primer hybridised to the end of the insert then labels the 1 kb segments through to a blocking oligonucleotide at the opposite end of the insert. This format prevents the M13 from becoming significantly labelled and allows the labelled material to be purified by denaturing reverse phase liquid chromatography for hybridization to the f X174 scaffold. One of the 1 kb segments is synthesized using a primer that contains the probe sequence as above. This has the advantage that only one probe sequence is present per labelled molecule. It is for the user to decide the combinations of labels that will be used with each particular segment and it will be appreciated that control of this aspect is absolute.

Two types of branched oligonucleotide have been used. The first are designed to be used as indexers in entirely the single stranded form but are able to anneal as complementary strands formed by Branched Indexer 1 with Branched Indexer 2. Shown below (see also separate sheet appended) are 2 examples of each Branched Indexer 1a and 1b with Branched Indexer 2a and 2b, respectively. The oligonucleotides are lined up with respect to their complementary regions which are the short ends either side of the branch. The long 5′ ends in each case provide a complementary strand for secondary detection with suitable hybridization probes. Hence each long 5′ end sequence is different. Their short 5′ ends are blocked, in this case with a hexylene glycol group to prevent participation in ligation. Indexer 1's have a longer 3′ end because this alone contains an AcuI site (underlined in sequence) for recutting in the target. The indexing ends are at the 8 bases at the 3′ ends with the core sequence italicised and the actual selective bases in bold. Note that 4 different indexing sequences have been used, 1 per branched oligonucleotide. These were for targeting bacteriophage lambda KpnI to EcoRV fragments of 712 bases for 1a with 2a and 2711 bases for 1b with 2b from positions 17058 to 17769 and 18561 to 21271 of the lambda genome, respectively. Appropriate modifications of the procedures described were used. Joining of the annealed strands is not possible without the filling reaction described above or through the use of a complementary filling oligonucleotide, also as described above and shown aligned BrFiIIL and BrFiIIR with the indexers below.

The single-stranded indexers take advantage of a PMOc branch introduced during oligonucleotide synthesis and also have 2 hexylene glycol spacers at the same position.

Branched oligonucleotides can also be formed through the use of a partially complementary oligonucleotide together with 2 complementary nucleotides. An example of indexer 1 formed in this way and having the general features including the AcuI site described above is shown below. It is composed of three oligonucleotides that have been annealed.

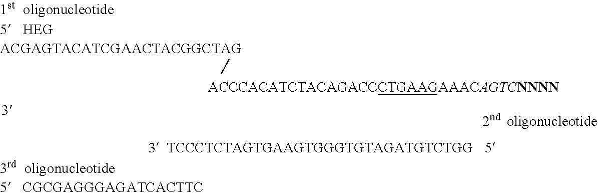

Note how the uppermost strand is free to hybridise for secondary detection. There is also a single stranded gap at the indexing end for filling with an oligonucleotide or by a polymerase as described above for creating the AcuI site. Having only one 3′ end for the indexing reaction enhances the fidelity. The third oligonucleotide anneals such that it retains a 4 base 5′ overhang for circularisation as described in the examples above.

Both types of branched oligonucleotide allow circularization. The first type 1 and 2 are single stranded and complementary to each other whilst the second type relies for circularization on a short (4 base) cohesive end at the end of its double stranded region.

The following sets of oligonucleotides have been developed to enable phiX174 to be used for either primary detection or secondary detection.

| | | |
|---|---|---|
| phiX350F | 5′ ctgagtccgatgctgttcaa | 3′ Spacer C3 |
| phiX350AS | 5′ ttgaacagcatcggactcag | |
| phiX1596R | 5′ gcagcttgcagacccataat | |
| phiX1718F | 5′ cgctctaatctctgggcatc | 3′ Spacer C3 |
| phiX1718AS | 5′ gatgcccagagattagagcg | |
| phiX2884R | 5′ cctgattcagcgaaaccaat | |
| phiX2938F | 5′ gtgctattgctggcggtatt | 3′ Spacer C3 |
| phiX2938AS | 5′ aataccgccagcaatagcac | |
| phiX4078R | 5′ gaaatgccacaagcctcaat | |
| phiX4082F | 5′ tctttctcaatccccaatgc | 3′ Spacer C3 |
| phiX4082AS | 5′ cgattggggattgagaaaga | |
| phiX5286R | 5′ ttcccagcctcaatctcatc | |
| phiXendF | 5′ cctgtgacgacaaatctgct | 3′ Spacer C3 |
| phiXendAS | 5′ agcagatttgtcgtcacagg | |
| phiXendR | 5′ ccagcagtccacttcgattt | |
| phiX5286KP17R | 5′ acgagtacatcgaactacggctagttcccagcctcaatctcatc | |
| phiX5286KP27R | 5′ ggagttctgcatcacttgacgctattcccagcctcaatctcatc | |
| phiX5286EV17R | 5′ tgtcggtgcgtcatttctaaggagttcccagcctcaatctcatc | |
| phiX5286EV21R | 5′ ggatccgaattcactcgggccaagttcccagcctcaatctcatc | |

In the case of the latter, two different labelling procedures have been adopted. They both take advantage, (optionally) of fragments of phiX174 cloned into bacteriophage M13. The first 5 sets of oligonucleotides each have a pair with a name ending F or R. Each of such pairs were used to PCR a different segment of phiX174 (nucleotide regions denoted in the number of the name. Note that labelled nucleotides can be used during PCR to create separate dye labelled segments. These can be annealed back to phiX174 as described above, thus labelling it uniformly according to a preferred code. Unlabelled segments were cloned separately into M13mp18 using standard procedures so that corresponding M13 single strand could be used for labelling the cloned segments. In this case the oligonucleotides with a name ending AS in the sets above were used as blocking primers (hence the 3′ spacer) with the corresponding name ending R primers during the labelling reaction. This had a number of advantages, polymerases like DNA polymeraseI Klenow Fragment (NEB) requiring less labelled nucleotide and therefore cheaper reactions could be used. In addition only one strand of phiX174 became labelled thus reducing probe reannealing and maximising the available probe. Finally, the different sizes of M13 and phiX174 allowed them to be more easily separated on size dependent purification.

The set with names having end in their middle span the PstI and BssH11 sites. These sites can then be cleaved for subsequent adaptoring so that the phiX174 can be used as a primary labelling as described in the examples above. In this case the polymerisation reactions are generally unlabelled. Note that the these are unique sites and in common with other unique sites present can be used to linearise the probe or indexer as appropriate.

Note that the 4 primers in the final set ending KP17R to EV21R substitute for phiX5286R. They have probe sequences at their 5′ end. In this case they detect the first type of branched oligonucleotides above and it will be seen that their probe sequences are complementary. They have the benefit of a single probe sequence per phiX174 molecule.

### EXAMPLE 9

### Single Molecule Visualisation

Visualisation of single molecules was by epifluorescence as described above. Many of the principles for spreading, alignment and imaging of single fluorescently-labelled DNA molecules have been established, through the development of optical mapping by Jing et al, 1988. Yokota H et al Nucleic Acids Res. 1997 25: 1064-1070 and Michalet X et al Science. 1997; 277:1518-1523, (incorporated herein by reference for all purposes) have used coated glass slides and mechanically-controlled meniscus motion to improve linear alignment. The Schwartz group have also developed optical mapping software. The indexing systems described above have great flexibility and are intended to work at a higher resolution than has been applied for optical mapping. The required resolution used by Femino et al is closer. Oligonucleotides ˜50 bases in length, labelled with ˜5 fluorophores, were readily imaged after in situ hybridization (ISH) at a resolution of 100-200 nm per pixel using techniques well known in the art. Furthermore, fluorophores about 1 Kb apart were resolved. The ISH images from the Singer group are similar in RNA contour length to the aligned DNA molecules imaged by Schwartz et al: about 3 Kb per μm. Thus the long indexers or our secondary detection probes should be at least 1 Kb for each base encoded and have at least 5 fluorophores per Kb. CCD cameras of 1.4 megapixels and a pixel size of 6.45 μm², on epifluorescent microscopes with 600× magnification meet the above 100-200 nm effective pixel size requirement.

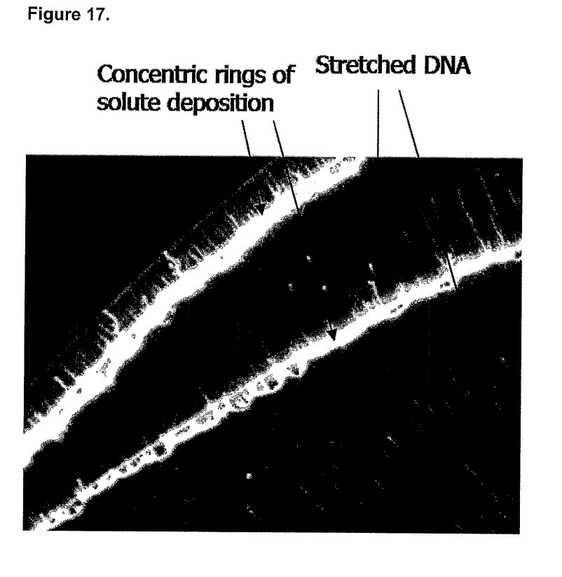

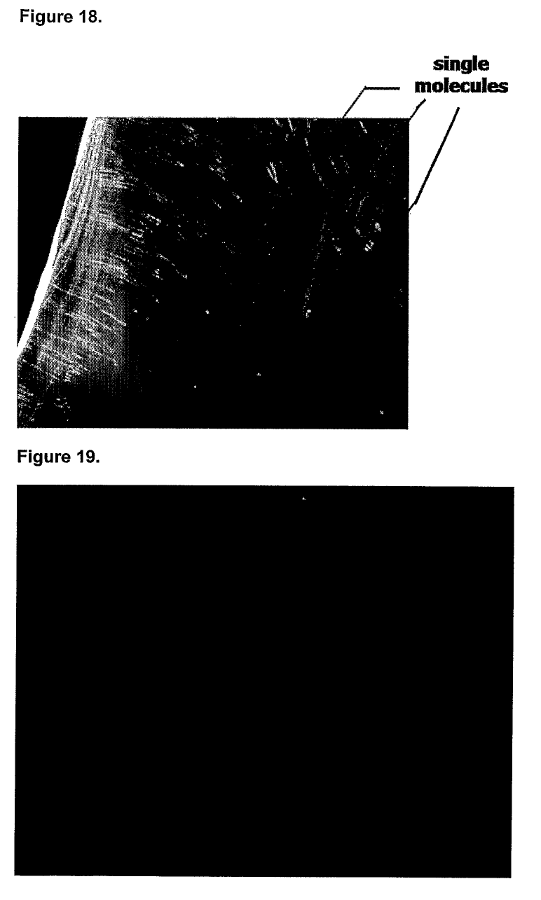

The optical mapping spreading procedure is very simple. In the case of non fluorescently labelled DNA to be detected by secondary detection sub μl spots of DNA solution in water or 10 mM Tris 1 mM EDTA pH 7.6 with or without 0.5% glycerol are allowed to dry at ambient temperature on pre-treated slides (APTES, Aldrich). FIG. 17, shows that DNA that has contacted the solid surface, is stretched at right angles to the meniscus as the latter moves by drying. A critical concentration of solutes is reached and material becomes deposited at the meniscus. Deposited material lowers the concentration of solutes and the process repeats so that low power images appear as a series of concentric rings. Provided that the DNA is adequately dilute then single molecules can be readily observed at right angles to the meniscus (see FIG. 18) once an appropriate concentration of solutes has been achieved. For this reason a series of DNA concentrations are spotted separately to determine the best conditions empirically. Shown in the FIG. 18 are molecules of bacteriophage lambda ˜50 Kb that have been spread and then stained with YOYO-1 (diluted in water to 100 nM, Molecular Probes). Note that the DNA molecules spread from the meniscus towards the centre of the droplet.

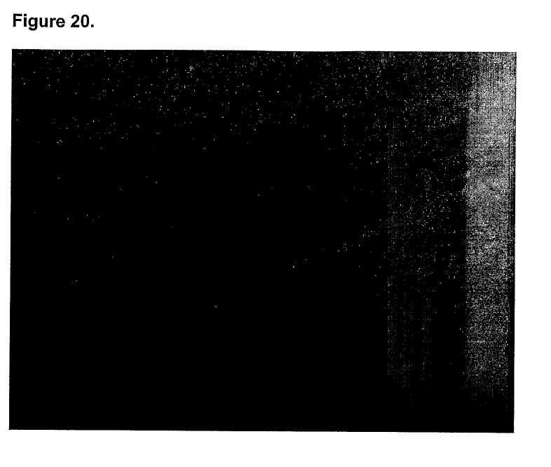

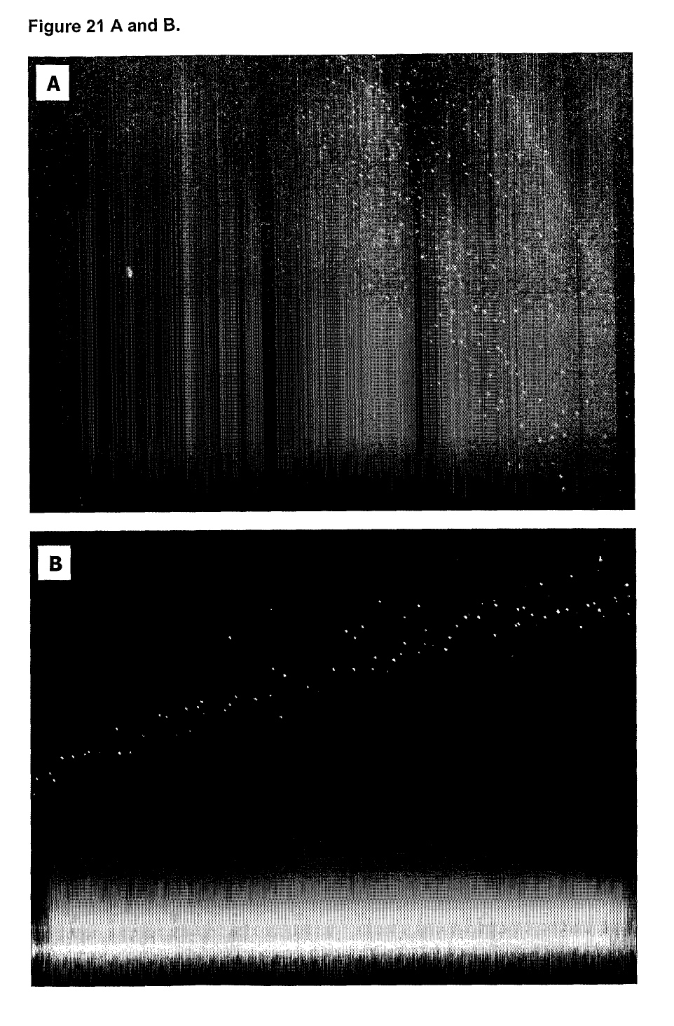

Indexers that had been directly labelled with fluorescent dyes (Alexa Fluor range, Molecular Probes) either by incorporation of labelled nucleotides or by coupling of succinimide esters to pre incorporated allyl dUTP failed to yield single molecules on the charged surface. Instead all material was deposited at the meniscus (see FIG. 19). This is a consequence of their hydrophobic nature. Inclusion of detergents improved the spreading. CHAPS at 5% was the best and others including deoxycholate and Triton X-100 at this concentration also worked. Concentric halos were produced as before (see FIG. 20). Labelled DNA now appeared as discrete spots or rods (see FIGS. 21a and 21b). This has advantages because it increases the number of molecules per field. It is also consistent with entirely spectrally encoded probes i.e. detection without regard to spatial resolution within an indexing molecule.

The distribution of sizes associated with the rings is notable. During drying, the detergents form a clear, circular deposit with obvious depth, almost like a frozen ripple. The apparent difference in sizes is caused by the refractive changes and associated magnifying effect of the surface created and also the differences in focal points that result. Combinations of labels, surfaces and detergents or other surfactants have to be determined empirically for particular requirements because for example some detergents have fluorescent emission spectra that overlap with some of the possible dyes and not all dyes behave in the same way for a given detergent. For example, Alexa Fluor 488 overlaps the fluorescence of CHAPS. Nor would indexers labelled with Alexa Fluor 488 yield single molecules when spread as described.

Methods adapted from Crut A et al Nucleic Acids Res. 2005; 33: e98 were preferable for indexers having hydrophobic labels. Crut et al used slides with a hydrophobic coat produced by spin coating with a polystyrene solution. Single molecules could be combed from dilute DNA solutions onto this surface. The single molecules are suitable for detection using quantum dots formatted in ways that closely resemble our new indexers and secondary detection probes. This is our preferred method for spreading. Slides were rigorously cleaned free of residues, baked dry and then spin coated at 1500 rpm for 2 minutes using a 5% solution of polystyrene in toluene (Sigma). DNA in solution in MES (Sigma) 5 mM, 1 mM EDTA pH 5.5 and labelled up to 1 hydrophobic dye molecule per 10 bases was spotted onto the slides in volumes between 0.2 ul and 6 ul. The spots were allowed to dry at ambient before visualizing. Results are very similar to those obtained with unlabelled DNA on a charged surface with DNA deposited at the shrinking meniscus in halos as evaporation leads to critical concentrations of solute. Suitable dilutions, determined empirically gave rise single molecules. There were two significant differences. Firstly, the larger volumes gave the best yield of single molecules and secondly the single molecules were perpendicular to the meniscus but radiated out in the opposite direction to the centre of the droplet. Hydrophobically labelled DNA is becoming attached to the surface and left behind by the shrinking meniscus. As its concentration increases it is deposited on mass hence larger spots are expected to give more single molecules because the concentration of solutes increases less quickly giving more opportunity for single molecules to attach and spread. The density of single molecules was noticeably higher than obtained by the combing as described by Crut et al. The best approach is therefore to have an optimum concentration of labelled DNA and not to allow dilution at all. This is similar to the mechanical methods. The method of Crut et al probably gives a lower yield because it is hard to comb the DNA from solution sufficiently slowly. We realized that an ideal non mechanical way of moving the meniscus is to place the surface to be coated into a reservoir of DNA solution and to allow the reservoir to drain from a suitably sized capillary. This has many benefits. It allows extremely slow drainage and therefore slow movement of the meniscus. Rates of movement can be varied and accurately controlled for optimized yield of attached single molecules. Drained sample can be collected and reused. The ratio of DNA solution to surface can be precisely controlled through the geometry of the reservoir. Varying the angle of the surface to be coated with respect to the vertical position allows the angle of the surface with respect to the meniscus to be precisely controlled for further optimization. Draining can optionally be controlled through pumping or a tap that can regulate the flow.

Several aspects of the invention are described above separately for the sake of clarity. Nevertheless, the skilled person will recognise that any of the methods of the invention, for example, creation of single stranded overhangs by engineering a nick site, lengthening of single stranded overhangs, bar-code end labelling of ds DNA molecules, controlled size reduction of labelled ds DNA molecules and adaptored sequencing may be combined in any way as required circumstances of the experiment under consideration. Accordingly, each and every aspect described herein is intended to be combined in any way as would seem appropriate and necessary to the skilled person with any other aspect disclosed herein. There is no intention for the disclosure to be limited to only those specific combinations highlighted or exemplified or referred to directly above. Unless it is not technically feasible, then any combination of the features described herein is contemplated. The invention should not be taken to be limited by the foregoing examples, which are intended to be merely illustrative.

### EXAMPLE 10

### Partial Resequencing of M13mp18 RF DNA Using Low Resolution Size Separation

M13 mp18 RF DNA (10 ug, NEB) was digested by DNAseI. Each 15 ul reaction contained 0.2 ug of DNA, 50 mM Tris HCl pH 7.6, 10 mM Manganese Chloride 0.1 mg/ml BSA (NEB) and 0.5 to 0.05 units of DNAseI (NEB). Reactions were performed at 37° C. for 20 minutes and immediately purified as standard above. A range of enzyme concentrations were used to produce a range of fragments between a few hundred bases and intact RF. Fragments were pooled for subsequent steps. Ends were repaired by T4 DNA polymerase and methylation protection using SssI, dam and AcuI methylases was performed as described above. An adapter for formatting blunt ends was added for indexing as described in Example 5.

The adapter was as follows:

| | |
|---|---|
| B1ProNA1_L | 5′ GACCTGAGAATCACAGACCCGGGATC |
| B1ProNA1_U | 3′ CTGGACTCTTAGTGTCTGGGCCCTAG |

Directionality was introduced into the M13 molecules by cutting to completion with KpnI and PstI (both NEB). The fragments were purified as standard and through a Chromaspin 200 spin column (Clontech) to remove the short, released KpnI to PstI fragment. The adapter below was added as standard above.

| | |
|---|---|
| BioGTAC_U | 5′ Biotin TEG ATTCGGCGAGCATCGGAAGTA |
| BioGTAC_L | 3′ TAAGCCGCTCGTAGCCTT |

having a 5′ hydrophobic group was ligated to the KpnI end using the standard T4 DNA ligase reaction. Unused adapters were removed by ultrafiltration and ion exchange chromatography as standard above. Blunt ends were prepared for indexing by cutting the N.AlwI and then the AvaI sites in the first adapter and the fragments purified for indexing also as above. Fragments ending at the KpnI site with the hydrophobic adapter were purified through C18 Genomix columns (Varian 1 ug per column). Columns were prepared with 50% acetonitrile, the sample loaded in EB, and the columns washed three times with 100 ul water each to material that had not received a hydrophobic adapter. Adaptored material was eluted with 50% acetonitrile and dried in a Speedivac (Howe). Samples were dissolved in 0.2×EB and indexed as standard above using indexers of the form 5′ XGATCNNNN where X was a PCRable sequence that also had a 5′ fluoroscein dye for direct detection. One indexer was used per reaction.

The indexers allowed the indexed fragments to be both PCR amplified using the primers for the indexer and for the hydrophobic adapter. PCR amplification was performed using conditions that favoured long range PCR, typically with Phusion DNA polymerase (NEB as recommended). Amplified fragments were analysed by 0.7% agarose gel electrophoresis. Fragments starting at the KpnI site and extending to the point of random cleavage by DNAseI and having received an indexer appropriate for their end were the predominant target for the amplification. Use of their known end sequence (corresponding to the indexer end), their relative size order and their approximate size allowed the sequence to be predicted at reads of >1 kilobase.

The fluoroscein label of the indexers also allowed indexed ends to be detected using the Gene Imagers system (GE healthcare). Reactions were analysed directly by agarose gel electrophoresis and Southern Blotting and detected as recommended by the supplier. Fragments starting at the KpnI site and extending to the point of random cleavage by DNAseI and having received an indexer appropriate for their end were the target for detection through the indexer mediated labelling. Knowledge of the fragment sizes and indexed ends were used as above to determine sequence.

1. A method of differentially labelling one end of a double stranded (ds) DNA molecule on the basis of its nucleic acid sequence, comprising:
(a) providing said ds DNA molecule in linear form with at least one single stranded overhanging end;
(b) incubating, under conditions suitable to allow for DNA ligation, said ds DNA molecule having at least one single stranded overhanging end with a pool of different indexing molecules, the different indexing molecules of the pool having complementary single stranded ends for annealing to the at least one overhanging end of the ds DNA molecule, said different indexing molecules being labelled and distinguishable from one another, to produce a linear ligation product having indexing molecules at each end thereof;
(c) circularising the linear ligation product of step (b) by incubating under conditions suitable to allow for DNA ligation;
(d) linearising the circular product of step (c) by cleavage with a restriction enzyme having a cleavage site that is physically displaced from its recognition site, said recognition site being present in a portion of the circular product which is not derived from the original ds DNA molecule to be labelled, and said cleavage site being located within a portion of said circular product which is derived from the original ds DNA molecule to be labelled, such that a linear DNA molecule is produced having single stranded overhanging ends with nucleotide sequences characteristic of the ds DNA molecule; and, optionally
(e) repeating steps (b) to (d) one or more times

2. A method as claimed in claim 1 wherein the ds DNA molecule is one of a plurality of DNA molecules in a mixture, said plurality of ds DNA molecules of the mixture having different nucleic acid sequences, wherein two or more of the plurality of ds DNA molecules are differentially labelled.

3. A method as claimed in claim 1 or claim 2 wherein both ends of the ds DNA molecule of step (a) are provided with single stranded overhanging ends.

4. A method as claimed in claim 1 or claim 2 wherein one end of the ds DNA molecule of step (a) is provided with a single stranded overhanging end and the opposite end is blunt ended.

5. A method of any preceding claim wherein the pool of indexing molecules comprises indexing molecule having all possible complementary single stranded ends of a predetermined length for annealing and ligating to all possible single stranded overhanging ends of the ds DNA molecule.

6. A method of any preceding claim wherein the restriction enzyme having a cleavage site that is physically displaced from its recognition site is a Type IIs restriction enzyme or an interrupted palindrome restriction enzyme.

7. A method for controlled size reduction of a ds DNA molecule labelled according to the methods of any preceding claim, comprising:
(a) incubating, under conditions suitable to allow for DNA ligation, said labelled ds DNA molecule with a pool of different adaptor molecules, the different adaptor molecules of the pool having:
(i) complementary single stranded ends for annealing to the overhanging end of the ds DNA molecule distal to the indexing molecule derived label thereof; and
(ii) a recognition site for a restriction enzyme having a cleavage site that is physically displaced from its recognition site
in order to provide a labelled ds DNA molecule/adaptor ligation product wherein the cleavage site for the restriction enzyme of step (a)(ii) is located within a portion of the labelled ds DNA molecule/adaptor ligation product derived from the labelled ds DNA molecule;
(b) cleaving said labelled ds DNA molecule/adaptor ligation product with said restriction enzyme of step (a)(ii) in order to remove the adaptor molecule and to reduce the length of the labelled ds DNA molecule by a controlled number of nucleic acid bases; and, optionally
(c) repeating steps (a) and (b) one or more times to achieve further controlled size reductions.

8. A method of claim 7 wherein said labelled ds DNA molecule is one of a plurality of labelled DNA molecules in a mixture, said plurality of labelled ds DNA molecules of the mixture having different nucleic acid sequences, and wherein the length of two or more of the plurality of labelled ds DNA molecules is reduced by a controlled number of bases.

9. A method of claim 7 or claim 8 wherein the pool of different adaptor molecules comprises adaptor molecules having all possible complementary single stranded ends of a predetermined length for annealing and ligating to all possible single stranded overhanging ends of the ds DNA molecule.

10. A method of any of claims 7 to 9 wherein the restriction enzyme having a cleavage site that is physically displaced from its recognition site is a Type IIs restriction enzyme or an interrupted palindrome restriction enzyme.

11. A method of determining one or more bases of a ds DNA molecule labelled according to a method of any of claims 1 to 6, comprising:
(a) incubating, under conditions suitable to allow for DNA ligation, said labelled ds DNA molecule with a pool of different sequencing adaptor molecules, the different sequencing adaptor molecules of the pool having complementary single stranded ends for annealing to the overhanging end of the ds DNA molecule distal to the indexing molecule derived label thereof, each of the different sequencing adaptor molecules being differentially labelled, in order to provide a labelled ds DNA molecule/sequencing adaptor ligation product; and
(b) detecting the sequencing adaptor ligated to the labelled ds DNA molecule in step (a) and determining one or more bases of the ds DNA molecule on the basis of the sequencing adaptor detected.

12. A method of claim 11 wherein the size of the labelled ds DNA molecule has been reduced in a controlled manner according to a method of any of claims 7 to 10.

13. A method of claim 11 or claim 12 wherein said labelled ds DNA molecule is one of a plurality of labelled DNA molecules in a mixture, said plurality of labelled ds DNA molecules of the mixture having different nucleic acid sequences, and wherein one or more nucleic acid bases are determined for two or more of said plurality of labelled ds DNA molecules.

14. A method for determining at least a partial nucleic acid sequence of two or more ds DNA molecules in a mixed population of ds DNA molecules having different nucleotide sequences, said method comprising:
(a) differentially labelling one end of said two or more ds DNA molecules in said mixed population according to a method as claimed in any of claims 1 to 6;
(b) conducting controlled size reduction of the differentially labelled ds DNA molecules in the mixed population according to a method of any of claims 7 to 10; and
(c) determining by adaptor mediated sequencing methods one or more nucleic acid bases for two or more of the labelled ds DNA molecules in a sample having undergone controlled size reduction.

15. A method as claimed in claim 14 wherein one or more nucleic acid bases is determined for said two or more labelled ds DNA molecules in at least two samples, each sample having undergone controlled size reduction of the labelled ds DNA molecules therein to a different extent.

16. A method of claim 15 wherein multiple rounds of controlled size reduction are carried out and said determining is carried out for each round of controlled size reduction.

17. A method of claim 15 wherein the mixed population of differentially labelled ds DNA molecules resulting from step (a) is separated into individual pools for controlled size reduction, each pool being subjected to controlled size reduction to a different extent.

18. A method of claim 15 wherein multiple rounds of controlled size reduction are carried out on a single pool, and that pool is sampled after at least one of the rounds of controlled size reduction for the determination of one or more nucleic acid bases for two or more of the labelled ds DNA molecules therein.

19. A method for determining at least a partial nucleic acid sequence of two or more ds DNA molecules in a mixed population of ds DNA molecules having different nucleotide sequences, said method comprising:
(a) differentially labelling one end of said two or more ds DNA molecules in said mixed population according to a method of any of claims 1 to 6;
(b) separating the product of step (a) into two or more separate pools for controlled size reduction of the labelled ds DNA molecules therein;
(c) conducting controlled size reduction on each of the separate pools created in step (b) according to a method of any of claims 7 to 10, wherein each separate pool is subjected to a different number of rounds of controlled size reduction; and
(d) for each separate pool of size reduced ds DNA molecules produced in step (c), determining one or more nucleic acid bases for two or more of the labelled ds DNA molecules using adaptor mediated sequencing.

20. A method of providing a single stranded overhanging end on a double stranded (ds) DNA molecule comprising engineering a nick in one strand of said ds DNA molecule, and dissociating away from said ds DNA molecule a single stranded fragment from said nicked strand, said fragment extending from the first end to the site of the nick.

21. A method of claim 20 wherein said engineering of the nick comprises ligating a double stranded adaptor molecule to the ds DNA molecule.

22. A method of claim 21 wherein said adaptor molecule is modified such that upon ligation to the ds DNA molecule only one strand of the adaptor molecule is covalently linked to the ds DNA molecule, thereby creating a nick in the other strand.

23. A method of claim 21 wherein said adaptor molecule comprises a recognition site for a nicking endonuclease and wherein following ligation, the nicking site for said nicking endonuclease is located in a portion of the ligation product derived from the ds DNA molecule, the method further comprising the step of incubating the ligation product with said nicking endonuclease to create a nick in one strand.

24. A method of claim 20 wherein the nick is engineered to be within 18 bases of the end of the ds DNA molecule such that an overhang of up to 18 bases results from the step of dissociation of the single stranded fragment.

25. A process for lengthening a single stranded overhanging end on a double stranded (ds) DNA molecule comprising:
(a) ligating to said single stranded overhanging end a lengthening adaptor molecule, said lengthening adaptor molecule having a recognition site for a nicking endonuclease, or being designed to create a recognition site for a nicking endonuclease on ligation to said ds DNA molecule, wherein the nick site for said nicking endonuclease following ligation to the ds DNA molecule is located within a portion of the ligation product derived from the ds DNA molecule;
(b) incubating the ligation product of step (a) with a nicking endonuclease that recognises said nicking endonuclease recognition site in order to nick one strand of the ds DNA molecule; and
(c) cleaving the lengthening adaptor in a predetermined position so that the ds DNA molecule remains ligated to a nucleotide sequence derived from the lengthening adaptor on the second strand, and so that the nucleotide sequence between the point of cleavage and the nick site on the first strand disassociates to leave a lengthened single stranded overhang.

26. A method of claim 25 wherein cleavage of the lengthening adaptor occurs before incubation with the nicking endonuclease.

27. A method of claim 25 wherein cleavage of the lengthening adaptor occurs after incubation with the nicking endonuclease.

28. A method of claim 25 wherein the lengthening adaptor is designed to include a recognition site for a restriction endonuclease to allow for cleavage by that enzyme.

29. A method of claim 25 wherein cleavage of the lengthening adaptor is brought about by the action of a restriction endonuclease.

30. A method of claim 25 wherein the lengthening adaptor contains dUTP at the predetermined cleavage site, and wherein cleavage is brought about by the action of uracil-DNA glycosylase.

31. A process for lengthening a single stranded overhanging end on a double stranded (ds) DNA molecule, comprising:
(a) ligating to said single stranded overhang a lengthening adaptor molecule, wherein said lengthening adaptor molecule:
(i) comprises a recognition site for a type II restriction endonuclease; and
(ii) comprises a recognition site for a nicking endonuclease between the type II endonuclease recognition site and the end of the lengthening adaptor to be ligated to the ds DNA molecule; or
(iii) is designed to create, on ligation to the ds DNA molecule, a recognition site for a nicking endonuclease between the type II endonuclease recognition site and the ds DNA molecule
and wherein the nick site for said nicking endonuclease, following ligation, is located within a portion of the ligation product derived from the ds DNA molecule;
(b) incubating the ligation product of step (a) with a nicking endonuclease that recognises said nicking endonuclease recognition site in order to nick one strand of the ds DNA molecule;
(c) incubating the nicked ligation product of step (b) with a type II restriction endonuclease that recognises said type II restriction endonuclease recognition site;
optionally wherein the order of steps (b) and (c) are reversed.

32. A method of any of claims 1 to 19 wherein at least one single stranded overhanging end for annealing and ligation to indexing molecules, adaptors or sequencing adaptors is created by a method of any of claims 20 to 24 or lengthened according to a method of any of claims 25 to 31.

33. A method of determining nucleic acid sequence of a DNA molecule comprising:
(a) random cleavage of the DNA molecule into fragments, e.g., by shearing or digestion with a restriction nuclease;
(b) filling in any cohesive ends of fragments produced in step (a) to create blunt ends;
(c) ligating a lengthening adapter to the ends of the fragments, said adapter having restriction sites being suitable for creating a lengthened cohesive end for indexing;
(d) cleaving the adapted fragments with a restriction endonuclease to provide a constant end shared by the adaptored fragments;
(e) ligating an adapter to said constant end, e.g. to provide a PCR primer binding site;
(f) cleaving the adapted fragments with restriction enzymes whose recognition sites are found in the lengthening adapter, so as to provide a lengthened cohesive for indexing distal to the adaptored constant end;
(g) indexing said lengthened cohesive end by ligation to a pool of sequencing adapters, said pool of sequencing adapters comprising adapters having cohesive ends complementary to possible cohesive ends exposed in the DNA molecule by the process of end lengthening, each of the different sequencing adapters having means for detectable differentiation from the other adapters in the pool; and
(h) separating the fragments by electrophoresis and determining the particular sequencing adapter ligated to fragments of different length.

34. A method as claimed in claim 33 wherein said means for detectable differentiation are distinguishable labels.

35. A method of claim 34 wherein said distinguishable labels are fluorescent labels.

36. A method of claims 33 to 35 wherein said means for detectable differentiation comprise a PCR primer binding site.

37. A method of claim 36 wherein determination of the sequencing adapter ligated to fragments of different lengths is by PCR analysis.

38. A method as claimed in any preceding claim wherein samples from multiple sources are analysed together in a multiplexed assay e.g., wherein an indexer that identifies the individual of origin is ligated to individual samples before pooling.
